SEQUENCE LISTING (744 sequences)

SEQ ID NO: 1 - DNA, 4536 bp, Artificial Sequence
  Scaffold sequence for logic gate 1 and logic gate 3
cattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtcacgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcaccttagcaagagccgcacgacgaccagaggccagttatccagagttaggatacctcaatgtgcatccgctcggttctaaggacattatttcagtcctttaagatctcccgtatagaagcctcacgttagggggcgccgtgccttcacgccctcccatttaggaataccttgtctccgccgtctttattcagtagccctatgcattacgatgtggcgcttcccccgcgtgggcgcagaaatttactgaggcggattcgaaacgactgtgagggcaggataggtgagcaggcactggcacgtaatcaaccaacggactcacccgtgtgcaggcctaaaaacagccctcgaagggcacttggatatgaatgaacccacttgttttgactcgtggaggcgtggttttattactgtgctcagttaacgccgcatgaatttagctctgatcaccgtaagggtaactgcactagacatgttgtgggcatttaagtcctgcagtatctttttgttaggtggaacggcctaggggtaccttccgtgagaaactcccagatgatgcatgttcgagtacttgtgaaatggatggtcgcatcccctcctctcacacattacactgtctcgcgcgggttgcgtcttgaccggtacaagttgtgtaaccttacacctctagaaacattttagcagtcgctccaattgataccacgacctcagcgcgcgttgggagaccgttgccaaacctagttttcatgttttccttcaaacgtgcgatataattcctcgcacctagccgcctagctgagaagtgttatataggtcacttgaaaagagtaggttcagcatatggaacttgggggtttgttcgctatgcggtaacgcgtgcccctctgacttgtcgatgactcccggcgcggtttcaatgcgggcatgtaatcgtcgcgaacctttactcaggctataaagcggcataaccagttgtaactaacagaatacagttctctctcaacggcaactacaaaatagcatacttgagacctacgacagtagggtcgcgtcaacctgtgcaagaaaaagctagaagagctgaattctgataattacagacccatacaccgattaaccccgacttataggcgtaagtggtgtcccttgcatgacccaggctatatctatgcccccatataaaccaggtcgacttcccactgatcgaaccaataaggaatgatcgcagcccataaagtacggttcaacgattgctgagcttcagtgaggttaacagcggctggtgcccgtacattgcctcgtcgattagcctagtgagcgtcgacaggagctttaactcctactttcatacgccaccgcacagacttcattgaccacgtatctgagactccttgtaaactgactactccagctccatcaacaatcttgaagaagttctatccgatcacggttatcgcgagtcttagcgtagccaatgctgggccgtagcgtctacttaacggaaagaagggtgaaagctgccacagaaaataaattactatttacatttgtaagacaaagttagtatggttaaatactggcttagtctgcgggaaacttagccatacgacttggtttgcctatcgttacagcgtacgacccgttcaccaatcgcatttgcgatgcagataacaactcggaagctgtatgacctctcgagcgttagtcgcagttttagtagagaaaacccgatagccaggcgttcaagcctattccccattatgcgtccgctattgacttcgctggaccctcgcggagtcggcgacaaataacatagaggatatcgtcacgggatagcgctctccattggaattgtacatgagagttggactgccgaacgcatgggtggagcagccaagcacccggggactacgaaggtttccgcgcatgtgttacctttcagccgaagagaaggtcggtggatctcgctctttcggagctcatactcctcatgctacaatgttaaaacgcaaaacctgactcaatctattgcccggtcgttggctcgcatgcttcctccttctgccccaatccgacatgactgagatgcgcttgatagcaatgaccaagatagtcgtctagattcatctttagcgacgtgacttagatttgcagaggaaacggggtgactgtagattatggcagttgatatctctccctgttatggagtgcggaggctgagtgctaatgttgccccgcaaagcagtgtcgttagaagtatatatgcagtggcggtgttcaatcaggatcagcccg

SEQ ID NO: 2 - DNA, 8064 bp, Artificial Sequence
  Scaffold sequence for logic gate 2 and for surface mimics
ggcaatgacctgatagcctttgtagatctctcaaaaatagctaccctctccggcattaatttatcagctagaacggttgaatatcatattgatggtgatttgactgtctccggcctttctcacccttttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttaatgctactactattagtagaattgatgccaccttttcagctcgcgccccaaatgaaaatatagctaaacaggttattgaccatttgcgaaatgtatctaatggtcaaactaaatctactcgttcgcagaattgggaatcaactgttatatggaatgaaacttccagacaccgtactttagttgcatatttaaaacatgttgagctacagcattatattcagcaattaagctctaagccatccgcaaaaatgacctcttatcaaaaggagcaattaaaggtactctctaatcctgacctgttggagtttgcttccggtctggttcgctttgaagctcgaattaaaacgcgatatttgaagtctttcgggcttcctcttaatctttttgatgcaatccgctttgcttctgactataatagtcagggtaaagacctgatttttgatttatggtcattctcgttttctgaactgtttaaagcatttgagggggattcaatgaatatttatgacgattccgcagtattggacgctatccagtctaaacattttactattaccccctctggcaaaacttcttttgcaaaagcctctcgctattttggtttttatcgtcgtctggtaaacgagggttatgatagtgttgctcttactatgcctcgtaattccttttggcgttatgtatctgcattagttgaatgtggtattcctaaatctcaactgatgaatctttctacctgtaataatgttgttccgttagttcgttttattaacgtagatttttcttcccaacgtcctgactggtataatgagccagttcttaaaatcgcataaggtaattcacaatgattaaagttgaaattaaaccatctcaagcccaatttactactcgttctggtgtttctcgtcagggcaagccttattcactgaatgagcagctttgttacgttgatttgggtaatgaatatccggttcttgtcaagattactcttgatgaaggtcagccagcctatgcgcctggtctgtacaccgttcatctgtcctctttcaaagttggtcagttcggttcccttatgattgaccgtctgcgcctcgttccggctaagtaacatggagcaggtcgcggatttcgacacaatttatcaggcgatgatacaaatctccgttgtactttgtttcgcgcttggtataatcgctgggggtcaaagatgagtgttttagtgtattcttttgcctctttcgttttaggttggtgccttcgtagtggcattacgtattttacccgtttaatggaaacttcctcatgaaaaagtctttagtcctcaaagcctctgtagccgttgctaccctcgttccgatgctgtctttcgctgctgagggtgacgatcccgcaaaagcggcctttaactccctgcaagcctcagcgaccgaatatatcggttatgcgtgggcgatggttgttgtcattgtcggcgcaactatcggtatcaagctgtttaagaaattcacctcgaaagcaagctgataaaccgatacaattaaaggctccttttggagccttttttttggagattttcaacgtgaaaaaattattattcgcaattcctttagttgttcctttctattctcactccgctgaaactgttgaaagttgtttagcaaaatcccatacagaaaattcatttactaacgtctggaaagacgacaaaactttagatcgttacgctaactatgagggctgtctgtggaatgctacaggcgttgtagtttgtactggtgacgaaactcagtgttacggtacatgggttcctattgggcttgctatccctgaaaatgagggtggtggctctgagggtggcggttctgagggtggcggttctgagggtggcggtactaaacctcctgagtacggtgatacacctattccgggctatacttatatcaaccctctcgacggcacttatccgcctggtactgagcaaaaccccgctaatcctaatccttctcttgaggagtctcagcctcttaatactttcatgtttcagaataataggttccgaaataggcagggggcattaactgtttatacgggcactgttactcaaggcactgaccccgttaaaacttattaccagtacactcctgtatcatcaaaagccatgtatgacgcttactggaacggtaaattcagagactgcgctttccattctggctttaatgaggatttatttgtttgtgaatatcaaggccaatcgtctgacctgcctcaacctcctgtcaatgctggcggcggctctggtggtggttctggtggcggctctgagggtggtggctctgagggtggcggttctgagggtggcggctctgagggaggcggttccggtggtggctctggttccggtgattttgattatgaaaagatggcaaacgctaataagggggctatgaccgaaaatgccgatgaaaacgcgctacagtctgacgctaaaggcaaacttgattctgtcgctactgattacggtgctgctatcgatggtttcattggtgacgtttccggccttgctaatggtaatggtgctactggtgattttgctggctctaattcccaaatggctcaagtcggtgacggtgataattcacctttaatgaataatttccgtcaatatttaccttccctccctcaatcggttgaatgtcgcccttttgtctttggcgctggtaaaccatatgaattttctattgattgtgacaaaataaacttattccgtggtgtctttgcgtttcttttatatgttgccacctttatgtatgtattttctacgtttgctaacatactgcgtaataaggagtcttaatcatgccagttcttttgggtattccgttattattgcgtttcctcggtttccttctggtaactttgttcggctatctgcttacttttcttaaaaagggcttcggtaagatagctattgctatttcattgtttcttgctcttattattgggcttaactcaattcttgtgggttatctctctgatattagcgctcaattaccctctgactttgttcagggtgttcagttaattctcccgtctaatgcgcttccctgtttttatgttattctctctgtaaaggctgctattttcatttttgacgttaaacaaaaaatcgtttcttatttggattgggataaataatatggctgtttattttgtaactggcaaattaggctctggaaagacgctcgttagcgttggtaagattcaggataaaattgtagctgggtgcaaaatagcaactaatcttgatttaaggcttcaaaacctcccgcaagtcgggaggttcgctaaaacgcctcgcgttcttagaataccggataagccttctatatctgatttgcttgctattgggcgcggtaatgattcctacgatgaaaataaaaacggcttgcttgttctcgatgagtgcggtacttggtttaatacccgttcttggaatgataaggaaagacagccgattattgattggtttctacatgctcgtaaattaggatgggatattatttttcttgttcaggacttatctattgttgataaacaggcgcgttctgcattagctgaacatgttgtttattgtcgtcgtctggacagaattactttaccttttgtcggtactttatattctcttattactggctcgaaaatgcctctgcctaaattacatgttggcgttgttaaatatggcgattctcaattaagccctactgttgagcgttggctttatactggtaagaatttgtataacgcatatgatactaaacaggctttttctagtaattatgattccggtgtttattcttatttaacgccttatttatcacacggtcggtatttcaaaccattaaatttaggtcagaagatgaaattaactaaaatatatttgaaaaagttttctcgcgttctttgtcttgcgattggatttgcatcagcatttacatatagttatataacccaacctaagccggaggttaaaaaggtagtctctcagacctatgattttgataaattcactattgactcttctcagcgtcttaatctaagctatcgctatgttttcaaggattctaagggaaaattaattaatagcgacgatttacagaagcaaggttattcactcacatatattgatttatgtactgtttccattaaaaaaggtaattcaaatgaaattgttaaatgtaattaattttgttttcttgatgtttgtttcatcatcttcttttgctcaggtaattgaaatgaataattcgcctctgcgcgattttgtaacttggtattcaaagcaatcaggcgaatccgttattgtttctcccgatgtaaaaggtactgttactgtatattcatctgacgttaaacctgaaaatctacgcaatttctttatttctgttttacgtgcaaataattttgatatggtaggttctaacccttccattattcagaagtataatccaaacaatcaggattatattgatgaattgccatcatctgataatcaggaatatgatgataattccgctccttctggtggtttctttgttccgcaaaatgataatgttactcaaacttttaaaattaataacgttcgggcaaaggatttaatacgagttgtcgaattgtttgtaaagtctaatacttctaaatcctcaaatgtattatctattgacggctctaatctattagttgttagtgctcctaaagatattttagataaccttcctcaattcctttcaactgttgatttgccaactgaccagatattgattgagggtttgatatttgaggttcagcaaggtgatgctttagatttttcatttgctgctggctctcagcgtggcactgttgcaggcggtgttaatactgaccgcctcacctctgttttatcttctgctggtggttcgttcggtatttttaatggcgatgttttagggctatcagttcgcgcattaaagactaatagccattcaaaaatattgtctgtgccacgtattcttacgctttcaggtcagaagggttctatctctgttggccagaatgtcccttttattactggtcgtgtgactggtgaatctgccaatgtaaataatccatttcagacgattgagcgtcaaaatgtaggtatttccatgagcgtttttcctgttgcaatggctggcggtaatattgttctggatattaccagcaaggccgatagtttgagttcttctactcaggcaagtgatgttattactaatcaaagaagtattgctacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgaattcgagctcggtacccggggatcctcaactgtgaggaggctcacggacgcgaagaacaggcacgcgtgctggcagaaacccccggtatgaccgtgaaaacggcccgccgcattctggccgcagcaccacagagtgcacaggcgcgcagtgacactgcgctggatcgtctgatgcagggggcaccggcaccgctggctgcaggtaacccggcatctgatgccgttaacgatttgctgaacacaccagtgtaagggatgtttatgacgagcaaagaaacctttacccattaccagccgcagggcaacagtgacccggctcataccgcaaccgcgcccggcggattgagtgcgaaagcgcctgcaatgaccccgctgatgctggacacctccagccgtaagctggttgcgtgggatggcaccaccgacggtgctgccgttggcattcttgcggttgctgctgaccagaccagcaccacgctgacgttctacaagtccggcacgttccgttatgaggatgtgctctggccggaggctgccagcgacgagacgaaaaaacggaccgcgtttgccggaacggcaatcagcatcgtttaactttacccttcatcactaaaggccgcctgtgcggctttttttacgggatttttttatgtcgatgtacacaaccgcccaactgctggcggcaaatgagcagaaatttaagtttgatccgctgtttctgcgtctctttttccgtgagagctatcccttcaccacggagaaagtctatctctcacaaattccgggactggtaaacatggcgctgtacgtttcgccgattgtttccggtgaggttatccgttcccgtggcggctccacctctgaaagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgctttgcctggtttccggcaccagaagcggtgccggaaagctggctggagtgcgatcttcctgaggccgatactgtcgtcgtcccctcaaactggcagatgcacggttacgatgcgcccatctacaccaacgtgacctatcccattacggtcaatccgccgtttgttcccacggagaatccgacgggttgttactcgctcacatttaatgttgatgaaagctggctacaggaaggccagacgcgaattatttttgatggcgttcctattggttaaaaaatgagctgatttaacaaaaatttaatgcgaattttaacaaaatattaacgtttacaatttaaatatttgcttatacaatcttcctgtttttggggcttttctgattatcaaccggggtacatatgattgacatgctagttttacgattaccgttcatcgattctcttgtttgctccagactctca

SEQ ID NO: 3 - DNA, 42 bp, Artificial Sequence
  Staple of logic gate 1_1
tttttttcctttttcaatgtgccacctaaattgtaagttttt

SEQ ID NO: 4 - DNA, 38 bp, Artificial Sequence
  Staple of logic gate 1_2
gcccgaccagccactggctgttgagatccagatcattg

SEQ ID NO: 5 - DNA, 34 bp, Artificial Sequence
  Staple of logic gate 1_3
tttttcagggttattggcccccgacgtcattttt

SEQ ID NO: 6 - DNA, 37 bp, Artificial Sequence
  Staple of logic gate 1_4
tctcatgagcggatacgtcttgagtccattatcaaaa

SEQ ID NO: 7 - DNA, 40 bp, Artificial Sequence
  Staple of logic gate 1_5
catatttgttccgcgcggcgaacggtttggaacaagagtc

SEQ ID NO: 8 - DNA, 44 bp, Artificial Sequence
  Staple of logic gate 1_6
ccgaaaaattaacccggtaagacgtgcacccagcgtttctgttt

SEQ ID NO: 9 - DNA, 42 bp, Artificial Sequence
  Staple of logic gate 1_7
tttttcgttaatattttgttagccaaagaacgtggacttttt

SEQ ID NO: 10 - DNA, 37 bp, Artificial Sequence
  Staple of logic gate 1_8
gctgcgcacgacacccccctggaagctccttctccct

SEQ ID NO: 11 - DNA, 39 bp, Artificial Sequence
  Staple of logic gate 1_9
ttttttttgttaaatcagctcatttcactattgcttacc

SEQ ID NO: 12 - DNA, 38 bp, Artificial Sequence
  Staple of logic gate 1_10
tttaaccactctcctgaccgccactgcatatacaactg

SEQ ID NO: 13 - DNA, 36 bp, Artificial Sequence
  Staple of logic gate 1_11
aaatcggcaaaatccttgagtgtcgggaaataaaca

SEQ ID NO: 14 - DNA, 45 bp, Artificial Sequence
  Staple of logic gate 1_12
ttgttccatggcgagaaaggaaggaggtgctagaaaattcgctcc

SEQ ID NO: 15 - DNA, 32 bp, Artificial Sequence
  Staple of logic gate 1_13
ttttttccaatttagagcttgacggaaaggga

SEQ ID NO: 16 - DNA, 53 bp, Artificial Sequence
  Staple of logic gate 1_14
actaaatcggaaccctggaaagccacatttccggatacctgacacggatgccg

SEQ ID NO: 17 - DNA, 41 bp, Artificial Sequence
  Staple of logic gate 1_15
tttttaagggcgaaaaaccgtctatcagggcgatggcccac

SEQ ID NO: 18 - DNA, 52 bp, Artificial Sequence
  Staple of logic gate 1_16
ctaactttgtggcattctaggaatagcctctggtttatatggggaggttttt

SEQ ID NO: 19 - DNA, 39 bp, Artificial Sequence
  Staple of logic gate 1_17
cccctaacgtgaccccgataacagcattgcactaggtga

SEQ ID NO: 20 - DNA, 47 bp, Artificial Sequence
  Staple of logic gate 1_18
tttttctaagtttggcttctatacgcccgcgcgagacagtcgtggta

SEQ ID NO: 21 - DNA, 57 bp, Artificial Sequence
  Staple of logic gate 1_19
gtaacgatattgagtcagatccaccgaccttctcttcggctgaaaggtaacattttt

SEQ ID NO: 22 - DNA, 46 bp, Artificial Sequence
  Staple of logic gate 1_20
cgtacgctttccgagtatctcagtcatgcgttcggcagtctatccc

SEQ ID NO: 23 - DNA, 46 bp, Artificial Sequence
  Staple of logic gate 1_21
tgcatcgctcctaaatatgtccttggttacacaacttgttttctag

SEQ ID NO: 24 - DNA, 58 bp, Artificial Sequence
  Staple of logic gate 1_22
ctatcgggttttttttttttttctctactaaaactgcgacggaagcatgcggattttt

SEQ ID NO: 25 - DNA, 26 bp, Artificial Sequence
  Staple of logic gate 1_23
gaacgtgcactccgcgagggtttttt

SEQ ID NO: 26 - DNA, 40 bp, Artificial Sequence
  Staple of logic gate 1_24
ggctccgcggccaggagctccacccatgtcggattggggc

SEQ ID NO: 27 - DNA, 31 bp, Artificial Sequence
  Staple of logic gate 1_25
ccccctgagtgacgatgtttcctctgcaaat

SEQ ID NO: 28 - DNA, 38 bp, Artificial Sequence
  Staple of logic gate 1_26
cgagcataacagggagagatattacttctagtaaccac

SEQ ID NO: 29 - DNA, 44 bp, Artificial Sequence
  Staple of logic gate 1_27
ataggcttcgctcctgattgaacttccgacggcaaaaaatgttg

SEQ ID NO: 30 - DNA, 50 bp, Artificial Sequence
  Staple of logic gate 1_28
tttttccagcgaagtcaatagcggaaatgcgggctgagttaaattttttt

SEQ ID NO: 31 - DNA, 56 bp, Artificial Sequence
  Staple of logic gate 1_29
cgcctacagtcaccccatcctctatgttatttgcttggctaccgtaaacgagaggt

SEQ ID NO: 32 - DNA, 46 bp, Artificial Sequence
  Staple of logic gate 1_30
tttttaaccttcgtagtccccgggtgtcgccgtggcgttatgggga

SEQ ID NO: 33 - DNA, 46 bp, Artificial Sequence
  Staple of logic gate 1_31
tttttcatgcgcgagccaacgaccgagaaggataacgctaaggccg

SEQ ID NO: 34 - DNA, 33 bp, Artificial Sequence
  Staple of logic gate 1_32
ggcaatagaggcaaaccaagtcgtatggttttt

SEQ ID NO: 35 - DNA, 36 bp, Artificial Sequence
  Staple of logic gate 1_33
cgttttacaagcgctgttatcaaaaggccagcaaaa

SEQ ID NO: 36 - DNA, 57 bp, Artificial Sequence
  Staple of logic gate 1_34
ttttttaaggatcttttaaatactcatactcacgctcagtggaacctaaattttttt

SEQ ID NO: 37 - DNA, 43 bp, Artificial Sequence
  Staple of logic gate 1_35
ctttcaccaactgacgcgccaaatgcttcgctcgtcgtttggt

SEQ ID NO: 38 - DNA, 50 bp, Artificial Sequence
  Staple of logic gate 1_36
ggaaagttgcctacgggaagcgcaggttgttgccattgctacaggttttt

SEQ ID NO: 39 - DNA, 52 bp, Artificial Sequence
  Staple of logic gate 1_37
tttttcgaccatccacccgctttcatttcacgtatttaaccatacgtcctgg

SEQ ID NO: 40 - DNA, 71 bp, Artificial Sequence
  Staple of logic gate 1_38
tttcgctacggccccgtgatcggttaaagctcaattggcgcgaccctactgtcgtaggtctcaagtttttt

SEQ ID NO: 41 - DNA, 34 bp, Artificial Sequence
  Staple of logic gate 1_39
ttttttggtcatgagattgaagcatttatttttt

SEQ ID NO: 42 - DNA, 48 bp, Artificial Sequence
  Staple of logic gate 1_40
aggatcttcagttacccatagcagtgcgcaacaagtggtcctgcaact

SEQ ID NO: 43 - DNA, 48 bp, Artificial Sequence
  Staple of logic gate 1_41
tggtctgacacctagaaacccactcacgacttatcgccacaatagggg

SEQ ID NO: 44 - DNA, 42 bp, Artificial Sequence
  Staple of logic gate 1_42
ttttttcagcgatctgtctattactcttcagcgatttttttt

SEQ ID NO: 45 - DNA, 48 bp, Artificial Sequence
  Staple of logic gate 1_43
cgagttgcaagtgctcttcgatgttccttttacttatccggtaactat

SEQ ID NO: 46 - DNA, 45 bp, Artificial Sequence
  Staple of logic gate 1_44
cggcgacctccatcaagtagttcgccagtattcagctagtaaact

SEQ ID NO: 47 - DNA, 31 bp, Artificial Sequence
  Staple of logic gate 1_45
ttttttcatccatcgaaaactcacgtttttt

SEQ ID NO: 48 - DNA, 42 bp, Artificial Sequence
  Staple of logic gate 1_46
tttttaataaacctgttatcactccagatttatcagcttttt

SEQ ID NO: 49 - DNA, 32 bp, Artificial Sequence
  Staple of logic gate 1_47
ctcatgggtaagttggccgcagagccattttt

SEQ ID NO: 50 - DNA, 30 bp, Artificial Sequence
  Staple of logic gate 1_48
ttactgtagctccttcggtcctttatccgc

SEQ ID NO: 51 - DNA, 31 bp, Artificial Sequence
  Staple of logic gate 1_49
tttttgccggaagggccgtaattcgtttttt

SEQ ID NO: 52 - DNA, 42 bp, Artificial Sequence
  Staple of logic gate 1_50
tttttcatcgtggtgtcaaatcagtgaggcacctatcttttt

SEQ ID NO: 53 - DNA, 48 bp, Artificial Sequence
  Staple of logic gate 1_51
atggcttctaatagttaactttaatcttgcccggcgtcaatgactccc

SEQ ID NO: 54 - DNA, 36 bp, Artificial Sequence
  Staple of logic gate 1_52
tttttggaggaagactcggcagactaagccattttt

SEQ ID NO: 55 - DNA, 39 bp, Artificial Sequence
  Staple of logic gate 1_53
gattgttgatggagcgaagtctgaggtgtaaagctcttc

SEQ ID NO: 56 - DNA, 40 bp, Artificial Sequence
  Staple of logic gate 1_54
accggtcattcttcaacgtgaaggcacggcgccatacagc

SEQ ID NO: 57 - DNA, 42 bp, Artificial Sequence
  Staple of logic gate 1_55
ttttttcccaacgcgcgctgaggtgtaatgtgtgagattttt

SEQ ID NO: 58 - DNA, 39 bp, Artificial Sequence
  Staple of logic gate 1_56
tcctgtcgttgccggcgccgggagtcatcgacaattttt

SEQ ID NO: 59 - DNA, 37 bp, Artificial Sequence
  Staple of logic gate 1_57
tgcggtggttacaattcgcgacgattacaaccactta

SEQ ID NO: 60 - DNA, 31 bp, Artificial Sequence
  Staple of logic gate 1_58
tttttaaactgctgctacgctggatgttttt

SEQ ID NO: 61 - DNA, 48 bp, Artificial Sequence
  Staple of logic gate 1_59
cgaggcaaaagtaggagatagaacagacgcaaggagatcttaaaggac

SEQ ID NO: 62 - DNA, 36 bp, Artificial Sequence
  Staple of logic gate 1_60
tgtacgggcaccagccggctagcgcctattccttat

SEQ ID NO: 63 - DNA, 32 bp, Artificial Sequence
  Staple of logic gate 1_61
aattatatcgcacgttctaatcgattgaaacc

SEQ ID NO: 64 - DNA, 32 bp, Artificial Sequence
  Staple of logic gate 1_62
aagtcgacgggtcatgcaagggactgcccgca

SEQ ID NO: 65 - DNA, 31 bp, Artificial Sequence
  Staple of logic gate 1_63
tttttgtcaggcatagataaacatgattttt

SEQ ID NO: 66 - DNA, 42 bp, Artificial Sequence
  Staple of logic gate 1_64
tttttatgctattttgtagacaggtttggcaacggtcttttt

SEQ ID NO: 67 - DNA, 40 bp, Artificial Sequence
  Staple of logic gate 1_65
ttgagagagaactgtacaggttgaagcgactgctaaaatg

SEQ ID NO: 68 - DNA, 31 bp, Artificial Sequence
  Staple of logic gate 1_66
tagctttttcttgcattctgttagcgtatga

SEQ ID NO: 69 - DNA, 47 bp, Artificial Sequence
  Staple of logic gate 1_67
tggcagcgctgcgcaattaaaaatgaagttttaaaaaactctttttt

SEQ ID NO: 70 - DNA, 47 bp, Artificial Sequence
  Staple of logic gate 1_68
aatgtattcgtaaagctacgtgaaccatcaccctaatcaagtttttt

SEQ ID NO: 71 - DNA, 42 bp, Artificial Sequence
  Staple of logic gate 1_69
gcgtggcgctttctcaaagatacctcaaaagaagggcttttt

SEQ ID NO: 72 - DNA, 40 bp, Artificial Sequence
  Staple of logic gate 1_70
aggcgttttgctttgcggggcaacattagaacccgttttt

SEQ ID NO: 73 - DNA, 42 bp, Artificial Sequence
  Staple of logic gate 1_71
tttttgctggcaagtgtagcggtcacctaagtcacactccat

SEQ ID NO: 74 - DNA, 34 bp, Artificial Sequence
  Staple of logic gate 1_72
tttttgcgctatagaccgagatagggcttataaa

SEQ ID NO: 75 - DNA, 32 bp, Artificial Sequence
  Staple of logic gate 1_73
ttttttttttggggtcggaagaaagtaggtcg

SEQ ID NO: 76 - DNA, 42 bp, Artificial Sequence
  Staple of logic gate 1_74
cacaaaaatcgacgcttcttgctacatgtacatagacttttt

SEQ ID NO: 77 - DNA, 42 bp, Artificial Sequence
  Staple of logic gate 1_75
ttttttggcgacactcagcctccgcgtcgctaaagatttttt

SEQ ID NO: 78 - DNA, 42 bp, Artificial Sequence
  Staple of logic gate 1_76
tttttgaatcattccaatggagagcgccaactctaggtgagc

SEQ ID NO: 79 - DNA, 63 bp, Artificial Sequence
  Staple of logic gate 1_77
tttttgactatcttggtcattgctatacattgtagattggtgaacgggttgaaatagggaggg

SEQ ID NO: 80 - DNA, 39 bp, Artificial Sequence
  Staple of logic gate 1_78
ttttttgaggagtatgagctccgaaagagcgaggttttg

SEQ ID NO: 81 - DNA, 67 bp, Artificial Sequence
  Staple of logic gate 1_79
tttttcaaggatcttaccgtaacaggattagcgaaccccccgttcaaagctgggctgtgtgcttttt

SEQ ID NO: 82 - DNA, 63 bp, Artificial Sequence
  Staple of logic gate 1_80
tttttgcgatcaatctaaagtgaaaacgtgctagagtcagtctattaattgttcccccttttt

SEQ ID NO: 83 - DNA, 32 bp, Artificial Sequence
  Staple of logic gate 1_81
aagcggttcatgccatccgtaagatgcttttt

SEQ ID NO: 84 - DNA, 31 bp, Artificial Sequence
  Staple of logic gate 1_82
tttttatgatgccgggaatcttcgggttttt

SEQ ID NO: 85 - DNA, 62 bp, Artificial Sequence
  Staple of logic gate 1_83
tttttttacaaggagtctagaaccgagcggatcggagacaaggtataaaataactctggtgc

SEQ ID NO: 86 - DNA, 31 bp, Artificial Sequence
  Staple of logic gate 1_84
ttttttggtcaattggagtagtcagtttttt

SEQ ID NO: 87 - DNA, 42 bp, Artificial Sequence
  Staple of logic gate 1_85
tttttccgtactttatgggcttttcaagtgacctgaattttt

SEQ ID NO: 88 - DNA, 34 bp, Artificial Sequence
  Staple of logic gate 1_86
agtaaaggctggttatgccgctttctgtattttt

SEQ ID NO: 89 - DNA, 31 bp, Artificial Sequence
  Staple of logic gate 1_87
tttttattatcagaattccagatacgttttt

SEQ ID NO: 90 - DNA, 66 bp, Artificial Sequence
  Staple of logic gate 1_88
tttttttttctgtccatctggccccctactctgcgatcataagtcggggttaatcggtgtattttt

SEQ ID NO: 91 - DNA, 40 bp, Artificial Sequence
  Staple of logic gate 1_89
tgtcagaattatggcagcactgcatcagtgggacccccaa

SEQ ID NO: 92 - DNA, 40 bp, Artificial Sequence
  Staple of logic gate 1_90
ccgatcgtcgtcgtgtaccgcgtgacccacgcgcgaacaa

SEQ ID NO: 93 - DNA, 30 bp, Artificial Sequence
  Staple of logic gate 1_91
gtgcgagggttccatatgctgaacagtgct

SEQ ID NO: 94 - DNA, 30 bp, Artificial Sequence
  Staple of logic gate 1_92
tttttggcacgcgttaccgcatatcaccgg

SEQ ID NO: 95 - DNA, 39 bp, Artificial Sequence
  Staple of logic gate 1_93
tggttcgataattctcgcaatgatagataactacgatac

SEQ ID NO: 96 - DNA, 30 bp, Artificial Sequence
  Staple of logic gate 1_94
cggaaggtacccctacagaaaacgagggct

SEQ ID NO: 97 - DNA, 58 bp, Artificial Sequence
  Staple of logic gate 1_95
atcctagtgcagttatcaaaaaagatactgcatgcggcgttacacgggtgtgtttgca

SEQ ID NO: 98 - DNA, 31 bp, Artificial Sequence
  Staple of logic gate 1_96
aaaccacgcctccacgtgcccttaaaggatc

SEQ ID NO: 99 - DNA, 42 bp, Artificial Sequence
  Staple of logic gate 1_97
tttttgtaggtatctcagttcggtgcgaaaggagcggttttt

SEQ ID NO: 100 - DNA, 32 bp, Artificial Sequence
  Staple of logic gate 1_98
tttttacaggactatatagctcacgctttttt

SEQ ID NO: 101 - DNA, 32 bp, Artificial Sequence
  Staple of logic gate 1_99
tttttcgtcgtgcggccaagtcagaggttttt

SEQ ID NO: 102 - DNA, 40 bp, Artificial Sequence
  Staple of logic gate 1_100
tttttaccaagtcattctgagaatagtgtatggggagggc

SEQ ID NO: 103 - DNA, 42 bp, Artificial Sequence
  Staple of logic gate 1_101
tttttatgttgtgcaaaattagactggtgagtactcattttt

SEQ ID NO: 104 - DNA, 40 bp, Artificial Sequence
  Staple of logic gate 1_102
tttttatcgttatataacacttctcagctaggcgctgtta

SEQ ID NO: 105 - DNA, 42 bp, Artificial Sequence
  Staple of logic gate 1_103
ttttttgggtatagcctgacctcactgaagctcagcattttt

SEQ ID NO: 106 - DNA, 32 bp, Artificial Sequence
  Staple of logic gate 1_104
ttttttggtagcggtggacttaaatgcttttt

SEQ ID NO: 107 - DNA, 45 bp, Artificial Sequence
  Staple of logic gate 1_105
gaacagtacggcaaattcttgaagttaccttcggaaaaagttttt

SEQ ID NO: 108 - DNA, 32 bp, Artificial Sequence
  Staple of logic gate 1_106
tttttgtgctacagagcaaaccaccgcttttt

SEQ ID NO: 109 - DNA, 51 bp, Artificial Sequence
  Staple of logic gate 1_107
tttttccacaacatgtgtaatgcatagttttttttttgctcacctatcctg

SEQ ID NO: 110 - DNA, 29 bp, Artificial Sequence
  Staple of logic gate 1_108
tttttgtgccagctaaattcaggtttttt

SEQ ID NO: 111 - DNA, 32 bp, Artificial Sequence
  Staple of logic gate 1_109
gttcattcatatccaagagtcaaagtttctca

SEQ ID NO: 112 - DNA, 51 bp, Artificial Sequence
  Staple of logic gate 1_110
tttataagggcgtccgcctctcgtgcgataggccgcacacccgccgcgctt

SEQ ID NO: 113 - DNA, 46 bp, Artificial Sequence
  Staple of logic gate 1_111
tttcgcccacgcgggggaagcgccacccctcacagtcgtttcgttt

SEQ ID NO: 114 - DNA, 45 bp, Artificial Sequence
  Staple of logic gate 1_112
ccataatatatttccatattattttctgtcttacaaatgtaattt

SEQ ID NO: 115 - DNA, 38 bp, Artificial Sequence
  Staple of logic gate 1_113
tttctctgctgaagccagtggtggcctaactacggttt

SEQ ID NO: 116 - DNA, 30 bp, Artificial Sequence
  Staple of logic gate 1_114
agtccgtcttgatctttggtatctgcgttt

SEQ ID NO: 117 - DNA, 39 bp, Artificial Sequence
  Staple of logic gate 1_115
tttaatccgcccccttacggtgatcagagtgcctttttt

SEQ ID NO: 118 - DNA, 51 bp, Artificial Sequence
  Strand with adapter for S_A_1
atatatgccggttcccaacgatcaaggcgagttactctattcccgacccta

SEQ ID NO: 119 - DNA, 43 bp, Artificial Sequence
  Strand with adapter for S_A_2
tttttagttggtagcttggttgattactctattcccgacccta

SEQ ID NO: 120 - DNA, 57 bp, Artificial Sequence
  Strand with adapter for S_A_3
gcacattttttttttttgaggtatcctaactggcctctggttctattcccgacccta

SEQ ID NO: 121 - DNA, 62 bp, Artificial Sequence
  Strand with adapter for S_B
tcaagaagatcctttgatctgggaacaagtggtaccttcactccactcacttcacttcacac

SEQ ID NO: 122 - DNA, 84 bp, Artificial Sequence
  Spring T30_1
caaaaaagggattttttttttttttttttttttttttttttctacactagaaagcagcagattacgcgggccgttccagtaata

SEQ ID NO: 123 - DNA, 80 bp, Artificial Sequence
  Spring T30_2
gtttttaggcctgcaactgagcacacctaacgtaaatttctgttttttttttttttttttttttttttttttatagtaat

SEQ ID NO: 124 - DNA, 94 bp, Artificial Sequence
  Spring T40_1
caaaaaagggattttttttttttttttttttttttttttttttttttttttctacactagaaagcagcagattacgcgggccgttccagtaata

SEQ ID NO: 125 - DNA, 90 bp, Artificial Sequence
  Spring T40_2
gtttttaggcctgcaactgagcacacctaacgtaaatttctgttttttttttttttttttttttttttttttttttttttttatagtaat

SEQ ID NO: 126 - DNA, 104 bp, Artificial Sequence
  Spring T50_1
caaaaaagggattttttttttttttttttttttttttttttttttttttttttttttttttctacactagaaagcagcagattacgcgggccgttccagtaata

SEQ ID NO: 127 - DNA, 100 bp, Artificial Sequence
  Spring T50_2
gtttttaggcctgcaactgagcacacctaacgtaaatttctgttttttttttttttttttttttttttttttttttttttttttttttttttatagtaat

SEQ ID NO: 128 - DNA, 75 bp, Artificial Sequence
  Spring ds21_1
caaaaaagggacgcaaactaacactaacatcactacactagaaagcagcagattacgcgggccgttccagtaata

SEQ ID NO: 129 - DNA, 71 bp, Artificial Sequence
  Spring ds21_2
gtttttaggcctgcaactgagcacacctaacgtaaatttctgatcctgtgctagccgttcccaatagtaat

SEQ ID NO: 130 - DNA, 21 bp, Artificial Sequence
  Spring ds21_3
tgatgttagtgttagtttgcg

SEQ ID NO: 131 - DNA, 21 bp, Artificial Sequence
  Spring ds21_4
tgggaacggctagcacaggat

SEQ ID NO: 132 - DNA, 95 bp, Artificial Sequence
  Spring T10ds21T10_1
caaaaaagggattttttttttcgcaaactaacactaacatcattttttttttctacactagaaagcagcagattacgcgggccgttccagtaata

SEQ ID NO: 133 - DNA, 91 bp, Artificial Sequence
  Spring T10ds21T10_2
gtttttaggcctgcaactgagcacacctaacgtaaatttctgttttttttttcgcaaactaacactaacatcattttttttttatagtaat

SEQ ID NO: 134 - DNA, 21 bp, Artificial Sequence
  Spring T10ds21T10_3
tgatgttagtgttagtttgcg

SEQ ID NO: 135 - DNA, 105 bp, Artificial Sequence
  Spring T15ds21T15_1
caaaaaagggatttttttttttttttcgcaaactaacactaacatcatttttttttttttttctacactagaaagcagcagattacgcgggccgttccagtaata

SEQ ID NO: 136 - DNA, 101 bp, Artificial Sequence
  Spring T15ds21T15_2
gtttttaggcctgcaactgagcacacctaacgtaaatttctgtttttttttttttttcgcaaactaacactaacatcatttttttttttttttatagtaat

SEQ ID NO: 137 - DNA, 21 bp, Artificial Sequence
  Spring T15ds21T15_3
tgatgttagtgttagtttgcg

SEQ ID NO: 138 - DNA, 40 bp, Artificial Sequence
  Staple of surface mimic A and B_1
tttttagtaagcagatagccgaagcaatacttcttttttt

SEQ ID NO: 139 - DNA, 46 bp, Artificial Sequence
  Staple of surface mimic A and B_2
acgcaaagacaccacgaaaatacatacataaaggtggcaacttttt

SEQ ID NO: 140 - DNA, 32 bp, Artificial Sequence
  Staple of surface mimic A and B_3
tttttatataaaagaagcaccgtaatcttttt

SEQ ID NO: 141 - DNA, 40 bp, Artificial Sequence
  Staple of surface mimic A and B_4
tttttctttttttattcattaaaggccaacagagattttt

SEQ ID NO: 142 - DNA, 47 bp, Artificial Sequence
  Staple of surface mimic A and B_5
ggtgaattatagcaatagctatctattattaaagcgtaagaattttt

SEQ ID NO: 143 - DNA, 46 bp, Artificial Sequence
  Staple of surface mimic A and B_6
tttttagtagcgacagaatcactgaacacccaaaccatcgatagca

SEQ ID NO: 144 - DNA, 32 bp, Artificial Sequence
  Staple of surface mimic A and B_7
tttttataaacagccattaccgaagccttttt

SEQ ID NO: 145 - DNA, 46 bp, Artificial Sequence
  Staple of surface mimic A and B_8
gcgttttcatcggcaggagaattaaagtttgcctttagcgtttttt

SEQ ID NO: 146 - DNA, 32 bp, Artificial Sequence
  Staple of surface mimic A and B_9
tttttcagactgtagcgccagaatggattttt

SEQ ID NO: 147 - DNA, 40 bp, Artificial Sequence
  Staple of surface mimic A and B_10
tttttgccagtaccctcagagccctcaaatatcaattttt

SEQ ID NO: 148 - DNA, 46 bp, Artificial Sequence
  Staple of surface mimic A and B_11
tttttaagcgcagtctctgaatttagcgaactaaatcctcattaaa

SEQ ID NO: 149 - DNA, 32 bp, Artificial Sequence
  Staple of surface mimic A and B_12
ttttttaaaccaagtagagcctaatttttttt

SEQ ID NO: 150 - DNA, 46 bp, Artificial Sequence
  Staple of surface mimic A and B_13
ggcttttgatgataccgcgaggcgttttaccgttccagtaattttt

SEQ ID NO: 151 - DNA, 32 bp, Artificial Sequence
  Staple of surface mimic A and B_14
tttttgcgtcatacatgaggtttagtattttt

SEQ ID NO: 152 - DNA, 40 bp, Artificial Sequence
  Staple of surface mimic A and B_15
tttttaagaactattaagaggctaagaaaccaccattttt

SEQ ID NO: 153 - DNA, 46 bp, Artificial Sequence
  Staple of surface mimic A and B_16
tttttccgccaccctcagaacgttcagctaatatcaccgtactcag

SEQ ID NO: 154 - DNA, 32 bp, Artificial Sequence
  Staple of surface mimic A and B_17
ttttttgagaatcgcccttatcattccttttt

SEQ ID NO: 155 - DNA, 39 bp, Artificial Sequence
  Staple of surface mimic A and B_18
acaacgccagccctcatagttagcgtaacttaatttttt

SEQ ID NO: 156 - DNA, 46 bp, Artificial Sequence
  Staple of surface mimic A and B_19
agccaccaccctcattaaacaacatcgccaccctcagaaccttttt

SEQ ID NO: 157 - DNA, 32 bp, Artificial Sequence
  Staple of surface mimic A and B_20
tttttgccaccctcagaattttttcacttttt

SEQ ID NO: 158 - DNA, 40 bp, Artificial Sequence
  Staple of surface mimic A and B_21
tttttgtagggcgatctaaagttattgctttgaatttttt

SEQ ID NO: 159 - DNA, 30 bp, Artificial Sequence
  Staple of surface mimic A and B_22
ccaaaaaataccgacggaattgcgaataat

SEQ ID NO: 160 - DNA, 32 bp, Artificial Sequence
  Staple of surface mimic A and B_23
tttttgttgaaaatctggctccaaaagttttt

SEQ ID NO: 161 - DNA, 32 bp, Artificial Sequence
  Staple of surface mimic A and B_24
tttttcggcttaggttaacgctcaacattttt

SEQ ID NO: 162 - DNA, 46 bp, Artificial Sequence
  Staple of surface mimic A and B_25
tttttgagcctttaattgtatcggtttatcataatggtttgaaaaa

SEQ ID NO: 163 - DNA, 40 bp, Artificial Sequence
  Staple of surface mimic A and B_26
tttttctttttagttaaaggccgttagattaagacttttt

SEQ ID NO: 164 - DNA, 38 bp, Artificial Sequence
  Staple of surface mimic A and B_27
tgccactacgaaggcaccattccattaaacgggtaaaa

SEQ ID NO: 165 - DNA, 40 bp, Artificial Sequence
  Staple of surface mimic A and B_28
tttttgctgagaagagtatctacaaaggctatcagttttt

SEQ ID NO: 166 - DNA, 39 bp, Artificial Sequence
  Staple of surface mimic A and B_29
tttttgaccaactttgaaagtacgaggaagtacctaaaa

SEQ ID NO: 167 - DNA, 32 bp, Artificial Sequence
  Staple of surface mimic A and B_30
ttttttattcatttcaacctgtagcagagtct

SEQ ID NO: 168 - DNA, 55 bp, Artificial Sequence
  Staple of surface mimic A and B_31
ttacctgagcgatagccttttgcgtcaaaatcataggtctgagagactacttttt

SEQ ID NO: 169 - DNA, 63 bp, Artificial Sequence
  Staple of surface mimic A and B_32
aatggaaaaatattttaaaacaggaagattgtaaagggtgtcataagggaaccgaactttttt

SEQ ID NO: 170 - DNA, 47 bp, Artificial Sequence
  Staple of surface mimic A and B_33
tgaatcgcataactatggtcgctgaggcttgcagggaacctcttttt

SEQ ID NO: 171 - DNA, 30 bp, Artificial Sequence
  Staple of surface mimic A and B_34
ttttttgtacagaccaagaactggcttttt

SEQ ID NO: 172 - DNA, 47 bp, Artificial Sequence
  Staple of surface mimic A and B_35
tttttaccaagttacaatgggaagtgagcgagtaacaacctggcctt

SEQ ID NO: 173 - DNA, 55 bp, Artificial Sequence
  Staple of surface mimic A and B_36
ttcgcctgttgtcgtctaccagtataaagccgggttatgcagaggcgaatttttt

SEQ ID NO: 174 - DNA, 31 bp, Artificial Sequence
  Staple of surface mimic A and B_37
ttttttcattataccagtcaaaacaactaaa

SEQ ID NO: 175 - DNA, 39 bp, Artificial Sequence
  Staple of surface mimic A and B_38
tttttttatcagatgatagctctcgctattacgccagct

SEQ ID NO: 176 - DNA, 30 bp, Artificial Sequence
  Staple of surface mimic A and B_39
tttttaaatctacgtgttttgccagttttt

SEQ ID NO: 177 - DNA, 55 bp, Artificial Sequence
  Staple of surface mimic A and B_40
tttttgaaggagcggaaacgtcagagcggatcaaacttagaagggatggcaattc

SEQ ID NO: 178 - DNA, 55 bp, Artificial Sequence
  Staple of surface mimic A and B_41
gcggaacagagactccaatcggctgtctttcatatttatcatattcctgattttt

SEQ ID NO: 179 - DNA, 31 bp, Artificial Sequence
  Staple of surface mimic A and B_42
tttttagggggtaatagtaattaaataggtg

SEQ ID NO: 180 - DNA, 31 bp, Artificial Sequence
  Staple of surface mimic A and B_43
tttttatcaacagttgaaacggcagcaaccg

SEQ ID NO: 181 - DNA, 55 bp, Artificial Sequence
  Staple of surface mimic A and B_44
atcaggtctttacctgcatcgagaattattctgaaacatgaaaggggtatttttt

SEQ ID NO: 182 - DNA, 30 bp, Artificial Sequence
  Staple of surface mimic A and B_45
ttttttagcgtccaagaaccagaccttttt

SEQ ID NO: 183 - DNA, 55 bp, Artificial Sequence
  Staple of surface mimic A and B_46
tttttaccctcaatcaaattgttaccagcggtgccggtgaaatcgttaaggaatt

SEQ ID NO: 184 - DNA, 55 bp, Artificial Sequence
  Staple of surface mimic A and B_47
tgctgaacaccaccctacgagcgtctttccaccgcactgtcagttggcaattttt

SEQ ID NO: 185 - DNA, 31 bp, Artificial Sequence
  Staple of surface mimic A and B_48
tttttggaagcaaactccaaaaccaaacaaa

SEQ ID NO: 186 - DNA, 39 bp, Artificial Sequence
  Staple of surface mimic A and B_49
ttttttacgtggcacagcctgagaatacgagccggaagc

SEQ ID NO: 187 - DNA, 49 bp, Artificial Sequence
  Staple of surface mimic A and B_50
caactaactgtatcccaaccgccaccctcagaaccgcctacaaattttt

SEQ ID NO: 188 - DNA, 30 bp, Artificial Sequence
  Staple of surface mimic A and B_51
tttttgagtacctttaataacctgtttttt

SEQ ID NO: 189 - DNA, 55 bp, Artificial Sequence
  Staple of surface mimic A and B_52
ttttttagaacccttcttgccgtactggtttgccccagccgcctggcacaatatt

SEQ ID NO: 190 - DNA, 31 bp, Artificial Sequence
  Staple of surface mimic A and B_53
tttttttagctatattttcatggcaccaatg

SEQ ID NO: 191 - DNA, 31 bp, Artificial Sequence
  Staple of surface mimic A and B_54
ttttttgattagtaataaggaggcagggagc

SEQ ID NO: 192 - DNA, 47 bp, Artificial Sequence
  Staple of surface mimic A and B_55
ccgttgtacaaagttaaaggtaaatattgacggaaaaagaaattttt

SEQ ID NO: 193 - DNA, 63 bp, Artificial Sequence
  Staple of surface mimic A and B_56
tttttgaaaaggtggcatcaattcaaacgtaggaataagtttattttggaaacgtaaatacct

SEQ ID NO: 194 - DNA, 55 bp, Artificial Sequence
  Staple of surface mimic A and B_57
tttttattttagacaggaacggtacgccagaagctaaacacatcactgacattct

SEQ ID NO: 195 - DNA, 39 bp, Artificial Sequence
  Staple of surface mimic A and B_58
taccaaaaacccgccgtcatttggggcgcgagctttttt

SEQ ID NO: 196 - DNA, 40 bp, Artificial Sequence
  Staple of surface mimic A and B_59
tttttaatactaaccaccacacattatgaccctgtttttt

SEQ ID NO: 197 - DNA, 32 bp, Artificial Sequence
  Staple of surface mimic A and B_60
tttttcggaaccctaacgattaaagggttttt

SEQ ID NO: 198 - DNA, 40 bp, Artificial Sequence
  Staple of surface mimic A and B_61
tttttcgggaagtccactatacgctgcgcgttttgttttt

SEQ ID NO: 199 - DNA, 32 bp, Artificial Sequence
  Staple of surface mimic A and B_62
tttttagcggtccacgaagcactaaatttttt

SEQ ID NO: 200 - DNA, 31 bp, Artificial Sequence
  Staple of surface mimic A and B_63
ggccaacgtaacaggtcaggattagattttt

SEQ ID NO: 201 - DNA, 40 bp, Artificial Sequence
  Staple of surface mimic A and B_64
tttttctttattaatgaatcttggaacaaggaagcttttt

SEQ ID NO: 202 - DNA, 32 bp, Artificial Sequence
  Staple of surface mimic A and B_65
tttttttccacacaacgagttgcagcattttt

SEQ ID NO: 203 - DNA, 40 bp, Artificial Sequence
  Staple of surface mimic A and B_66
tttttacgcatctgccagcagccagctgcatttcattttt

SEQ ID NO: 204 - DNA, 32 bp, Artificial Sequence
  Staple of surface mimic A and B_67
tttttgttacctgcagtccgctcacaattttt

SEQ ID NO: 205 - DNA, 31 bp, Artificial Sequence
  Staple of surface mimic A and B_68
gcggtatggtaaatgtttagactggattttt

SEQ ID NO: 206 - DNA, 40 bp, Artificial Sequence
  Staple of surface mimic A and B_69
tttttaaaaagggcgcggttaccgggggttaggatttttt

SEQ ID NO: 207 - DNA, 32 bp, Artificial Sequence
  Staple of surface mimic A and B_70
ttttttctggtcagcatcagatgccggttttt

SEQ ID NO: 208 - DNA, 32 bp, Artificial Sequence
  Staple of surface mimic A and B_71
caagaatgcttgtagattacaaaaaacgctca

SEQ ID NO: 209 - DNA, 40 bp, Artificial Sequence
  Staple of surface mimic A and B_72
tttttagaacgttaaacgattcaatccgcctttttttttt

SEQ ID NO: 210 - DNA, 32 bp, Artificial Sequence
  Staple of surface mimic A and B_73
tttttgacgcagaaaccgtggtgctggttttt

SEQ ID NO: 211 - DNA, 31 bp, Artificial Sequence
  Staple of surface mimic A and B_74
ggaacggacagggacgttgggaagaattttt

SEQ ID NO: 212 - DNA, 40 bp, Artificial Sequence
  Staple of surface mimic A and B_75
ttttttatatagccgccacggaagggtaaacctcattttt

SEQ ID NO: 213 - DNA, 32 bp, Artificial Sequence
  Staple of surface mimic A and B_76
tttttcgggcctcttcacggaaaaagattttt

SEQ ID NO: 214 - DNA, 40 bp, Artificial Sequence
  Staple of surface mimic A and B_77
tttttatgcaacgacgacagtcagaggtggtttaattttt

SEQ ID NO: 215 - DNA, 32 bp, Artificial Sequence
  Staple of surface mimic A and B_78
tttttaacattaaatgggcgatcggtgttttt

SEQ ID NO: 216 - DNA, 31 bp, Artificial Sequence
  Staple of surface mimic A and B_79
taaacgttcagaggacagatgaacggttttt

SEQ ID NO: 217 - DNA, 40 bp, Artificial Sequence
  Staple of surface mimic A and B_80
ttttttgagtatttaaattggtttgaggggatgccttttt

SEQ ID NO: 218 - DNA, 32 bp, Artificial Sequence
  Staple of surface mimic A and B_81
tttttgtcattgcctgcagctttcatcttttt

SEQ ID NO: 219 - DNA, 40 bp, Artificial Sequence
  Staple of surface mimic A and B_82
tttttgtaggtaaagattcaataagcaaataatgtttttt

SEQ ID NO: 220 - DNA, 43 bp, Artificial Sequence
  Staple of surface mimic A and B_83
gaaacgcagtctgtctcaaacttcctcgaagccggcgaacgtg

SEQ ID NO: 221 - DNA, 37 bp, Artificial Sequence
  Staple of surface mimic A and B_84
aataataaaaagggcgacattcatgagccagtcacac

SEQ ID NO: 222 - DNA, 48 bp, Artificial Sequence
  Staple of surface mimic A and B_85
cggaatacccaaaagaactggcataatcatacgaacaatatggttgct

SEQ ID NO: 223 - DNA, 48 bp, Artificial Sequence
  Staple of surface mimic A and B_86
tatgttagctactaataaaaacgccgcttaatggcaagtgtagcggtc

SEQ ID NO: 224 - DNA, 40 bp, Artificial Sequence
  Staple of surface mimic A and B_87
aaattcatatggtttacaccagtaaaatggatgaacgagt

SEQ ID NO: 225 - DNA, 36 bp, Artificial Sequence
  Staple of surface mimic A and B_88
gtcacaacaacaggagtagtaaagctaaatcggttg

SEQ ID NO: 226 - DNA, 48 bp, Artificial Sequence
  Staple of surface mimic A and B_89
atcaccgtcaccgactaccgattgtagaagaaccatcacgaagtgttt

SEQ ID NO: 227 - DNA, 43 bp, Artificial Sequence
  Staple of surface mimic A and B_90
aagcccaataataagttttttggctcaactctttaaggcaaca

SEQ ID NO: 228 - DNA, 40 bp, Artificial Sequence
  Staple of surface mimic A and B_91
tatcagagagataaccagcctttattagagctatcgccat

SEQ ID NO: 229 - DNA, 48 bp, Artificial Sequence
  Staple of surface mimic A and B_92
tgaacaaagtcagaggggaagcgccttttgatgcagaagacgcgggga

SEQ ID NO: 230 - DNA, 31 bp, Artificial Sequence
  Staple of surface mimic A and B_93
caaaaatgaaaatagccacaagaaattctgc

SEQ ID NO: 231 - DNA, 48 bp, Artificial Sequence
  Staple of surface mimic A and B_94
cctcagagtccaaataagaaacgaagcaagaagaagtttcgaccagta

SEQ ID NO: 232 - DNA, 44 bp, Artificial Sequence
  Staple of surface mimic A and B_95
ccatcttttcataatcggaggttggcaacagtaagcccgatgag

SEQ ID NO: 233 - DNA, 32 bp, Artificial Sequence
  Staple of surface mimic A and B_96
cagagccgccaccagaaccgcctcgctattag

SEQ ID NO: 234 - DNA, 51 bp, Artificial Sequence
  Staple of surface mimic A and B_97
accaccaatgaaaacaaagcgtaatcatatccagcgcagtgtccataaaca

SEQ ID NO: 235 - DNA, 46 bp, Artificial Sequence
  Staple of surface mimic A and B_98
gccgccagcattgacaaaaatcacctaaaactaattgcaaatccct

SEQ ID NO: 236 - DNA, 43 bp, Artificial Sequence
  Staple of surface mimic A and B_99
cagacgattggccttccttattaccaccaaagaggtatagggt

SEQ ID NO: 237 - DNA, 48 bp, Artificial Sequence
  Staple of surface mimic A and B_100
gatattcaagaggtgaattcgagctgttcttcccgttttcacggtcat

SEQ ID NO: 238 - DNA, 40 bp, Artificial Sequence
  Staple of surface mimic A and B_101
ctcccgacttgcgggatccggtataatcgtcaatttagaa

SEQ ID NO: 239 - DNA, 32 bp, Artificial Sequence
  Staple of surface mimic A and B_102
aggagtgtactggtaatagcccgggactttac

SEQ ID NO: 240 - DNA, 28 bp, Artificial Sequence
  Staple of surface mimic A and B_103
ttatcaaagcgagaaaacaatggtcatt

SEQ ID NO: 241 - DNA, 40 bp, Artificial Sequence
  Staple of surface mimic A and B_104
tgcagaacgcgcctgtgtccagaccgaactaaacgtaaaa

SEQ ID NO: 242 - DNA, 46 bp, Artificial Sequence
  Staple of surface mimic A and B_105
caccagtaccagtaataagagaataaataatctcgtttagcccgaa

SEQ ID NO: 243 - DNA, 40 bp, Artificial Sequence
  Staple of surface mimic A and B_106
tttccagacgttagtagtagcattaatcctgaactaatgc

SEQ ID NO: 244 - DNA, 43 bp, Artificial Sequence
  Staple of surface mimic A and B_107
aatgaatacatcggcaccagagcgccatgggaacaaacggcgg

SEQ ID NO: 245 - DNA, 48 bp, Artificial Sequence
  Staple of surface mimic A and B_108
aactttcagtcagatgtggtttaagcaccgcttaggtcacgttggtgt

SEQ ID NO: 246 - DNA, 29 bp, Artificial Sequence
  Staple of surface mimic A and B_109
agcggagtgagaatacccaatatatttgc

SEQ ID NO: 247 - DNA, 48 bp, Artificial Sequence
  Staple of surface mimic A and B_110
gaaaggaataaagaaataccttattcaggaagaaccgtgcatctgcca

SEQ ID NO: 248 - DNA, 39 bp, Artificial Sequence
  Staple of surface mimic A and B_111
aaaaagcctgggattttgctaaacgtttcgtaatggaag

SEQ ID NO: 249 - DNA, 53 bp, Artificial Sequence
  Staple of surface mimic A and B_112
ttactaggcttgagaaatatacaacgccagtgtttaccagtcccgcggttgtg

SEQ ID NO: 250 - DNA, 32 bp, Artificial Sequence
  Staple of surface mimic A and B_113
cgtgtgataaataaggtcttctgaaggctggc

SEQ ID NO: 251 - DNA, 48 bp, Artificial Sequence
  Staple of surface mimic A and B_114
atgtaaatgctgatgcgcgttataacgagaaagagaaacaggggatgt

SEQ ID NO: 252 - DNA, 29 bp, Artificial Sequence
  Staple of surface mimic A and B_115
ttttagttaatttcacgttaaatgtgaat

SEQ ID NO: 253 - DNA, 48 bp, Artificial Sequence
  Staple of surface mimic A and B_116
cctaaattgcttgctttcgaggtgtttttcatataaatcaacggtcaa

SEQ ID NO: 254 - DNA, 43 bp, Artificial Sequence
  Staple of surface mimic A and B_117
aatttctctttttttgaccttgcatcgtatcgcactccagcca

SEQ ID NO: 255 - DNA, 36 bp, Artificial Sequence
  Staple of surface mimic A and B_118
cagcgaacccttagcctgatataatgccggagaggg

SEQ ID NO: 256 - DNA, 38 bp, Artificial Sequence
  Staple of surface mimic A and B_119
tcggaacgagggtagccgccgacttaattacccggata

SEQ ID NO: 257 - DNA, 39 bp, Artificial Sequence
  Staple of surface mimic A and B_120
aacggctacttctgtactccatgtatatgatattcaacc

SEQ ID NO: 258 - DNA, 40 bp, Artificial Sequence
  Staple of surface mimic A and B_121
aagtacaacggagttaggatcgtcaccctcagcataaccg

SEQ ID NO: 259 - DNA, 38 bp, Artificial Sequence
  Staple of surface mimic A and B_122
catctttgacccccagcgacctgaatcgtccaatcata

SEQ ID NO: 260 - DNA, 36 bp, Artificial Sequence
  Staple of surface mimic A and B_123
cgaaagaggcaaaagggcgcagatatatgagcccca

SEQ ID NO: 261 - DNA, 31 bp, Artificial Sequence
  Staple of surface mimic A and B_124
cggaacgaaatacactttgaggactaaagac

SEQ ID NO: 262 - DNA, 48 bp, Artificial Sequence
  Staple of surface mimic A and B_125
aatccttgtcgatgaacaaaaataattcgcgtccgtcggatcattcag

SEQ ID NO: 263 - DNA, 48 bp, Artificial Sequence
  Staple of surface mimic A and B_126
agatgatgaaagctgcttctccgttcgccattcaggctgcggcgaaag

SEQ ID NO: 264 - DNA, 29 bp, Artificial Sequence
  Staple of surface mimic A and B_127
aaacaaaacgccatcggtaatcgtaaaac

SEQ ID NO: 265 - DNA, 48 bp, Artificial Sequence
  Staple of surface mimic A and B_128
atttaacagttaaatctgtaccccggttgatacaccatcatacttagc

SEQ ID NO: 266 - DNA, 40 bp, Artificial Sequence
  Staple of surface mimic A and B_129
tttcaactgtaatcttcaaatatattgataccgatagttg

SEQ ID NO: 267 - DNA, 48 bp, Artificial Sequence
  Staple of surface mimic A and B_130
ataacggaatcaatatccacagacaacatgtaatttaggcgtagaaac

SEQ ID NO: 268 - DNA, 45 bp, Artificial Sequence
  Staple of surface mimic A and B_131
tacctttttttctgtatgtttagtatcatataaatccaacgtaac

SEQ ID NO: 269 - DNA, 48 bp, Artificial Sequence
  Staple of surface mimic A and B_132
ggtttaacacagtttcaacaccggaatcataaaactttttgacaagaa

SEQ ID NO: 270 - DNA, 37 bp, Artificial Sequence
  Staple of surface mimic A and B_133
ttgcgtaaaacgacccggaaacaatcggcaaaaaaag

SEQ ID NO: 271 - DNA, 38 bp, Artificial Sequence
  Staple of surface mimic A and B_134
atcagtttacatcgatggcagcctccggccacgcaacc

SEQ ID NO: 272 - DNA, 48 bp, Artificial Sequence
  Staple of surface mimic A and B_135
aaagattcggttagaacagcgccaggttttcccagtcacggctttccg

SEQ ID NO: 273 - DNA, 32 bp, Artificial Sequence
  Staple of surface mimic A and B_136
acaggtagccgacaaatgtaccgtaacactga

SEQ ID NO: 274 - DNA, 37 bp, Artificial Sequence
  Staple of surface mimic A and B_137
cggaacaccgcacagcaaacgcggtccgtatcagcgg

SEQ ID NO: 275 - DNA, 32 bp, Artificial Sequence
  Staple of surface mimic A and B_138
taataaaagacgacaatttcagggatagcaag

SEQ ID NO: 276 - DNA, 48 bp, Artificial Sequence
  Staple of surface mimic A and B_139
gaggcatagtaacattagcaccgtcactggtgtgttcagcccccctgc

SEQ ID NO: 277 - DNA, 45 bp, Artificial Sequence
  Staple of surface mimic A and B_140
gtaagagtcctcatccgccagcagttggggaatttgtgattatac

SEQ ID NO: 278 - DNA, 48 bp, Artificial Sequence
  Staple of surface mimic A and B_141
ccagacgactcgtcgccataaaaaaatcccgtgaaacgtacctaccat

SEQ ID NO: 279 - DNA, 32 bp, Artificial Sequence
  Staple of surface mimic A and B_142
ggcttttgccgttccgggcggcctttagtgat

SEQ ID NO: 280 - DNA, 40 bp, Artificial Sequence
  Staple of surface mimic A and B_143
tcgtattacgtcgagaaagtcctgaacaagaaataaagta

SEQ ID NO: 281 - DNA, 48 bp, Artificial Sequence
  Staple of surface mimic A and B_144
ttagagcctgggtaaagctggaggtgtccagctttttcgtcgataaaa

SEQ ID NO: 282 - DNA, 36 bp, Artificial Sequence
  Staple of surface mimic A and B_145
gaccataaatatcttcccttacggtggtgccatccc

SEQ ID NO: 283 - DNA, 48 bp, Artificial Sequence
  Staple of surface mimic A and B_146
tccccctcgcaaatcatcagtgccttgagtaaataagtgcaatccttt

SEQ ID NO: 284 - DNA, 46 bp, Artificial Sequence
  Staple of surface mimic A and B_147
atctaaagtaaagcctgctgattgcccttcacaggcgaaaaatatg

SEQ ID NO: 285 - DNA, 38 bp, Artificial Sequence
  Staple of surface mimic A and B_148
agcagcaaccagagcctacaattttatcctgtattttc

SEQ ID NO: 286 - DNA, 38 bp, Artificial Sequence
  Staple of surface mimic A and B_149
gccacgcctcacatttttttcttttcaccaaatcggca

SEQ ID NO: 287 - DNA, 48 bp, Artificial Sequence
  Staple of surface mimic A and B_150
caccgcctaggcaggtaatcaagattagttgcaatagcaaaaatgctt

SEQ ID NO: 288 - DNA, 46 bp, Artificial Sequence
  Staple of surface mimic A and B_151
agtattaaccgaacgaagcgtttgataacataaaaacaggtaattg

SEQ ID NO: 289 - DNA, 29 bp, Artificial Sequence
  Staple of surface mimic A and B_152
ggcggtctttccaggaggcggtttgcgta

SEQ ID NO: 290 - DNA, 32 bp, Artificial Sequence
  Staple of surface mimic A and B_153
atgttttaatcctgttaaatcaagttttttgg

SEQ ID NO: 291 - DNA, 47 bp, Artificial Sequence
  Staple of surface mimic A and B_154
tgaatatatgattcccattgagttttagagccagcaaaatccagcgc

SEQ ID NO: 292 - DNA, 45 bp, Artificial Sequence
  Staple of surface mimic A and B_155
attccatatcaccctgatggtggttccgagtgagacgtgcgcgaa

SEQ ID NO: 293 - DNA, 36 bp, Artificial Sequence
  Staple of surface mimic A and B_156
ttagataacattttgggcgctgcgccgctacagggc

SEQ ID NO: 294 - DNA, 32 bp, Artificial Sequence
  Staple of surface mimic A and B_157
catttcgcgtggactctgagtgttgttccagt

SEQ ID NO: 295 - DNA, 46 bp, Artificial Sequence
  Staple of surface mimic A and B_158
tatttacggaagggattgacgagcacgtatccagaattagcaaaat

SEQ ID NO: 296 - DNA, 31 bp, Artificial Sequence
  Staple of surface mimic A and B_159
aatatccaaggcaaggcaaaccgagtaaaag

SEQ ID NO: 297 - DNA, 31 bp, Artificial Sequence
  Staple of surface mimic A and B_160
agccattgtcaatagactccttattacgcag

SEQ ID NO: 298 - DNA, 47 bp, Artificial Sequence
  Staple of surface mimic A and B_161
ttataatcagtgaggaacgtgcttatcggccattcaccatttgggaa

SEQ ID NO: 299 - DNA, 48 bp, Artificial Sequence
  Staple of surface mimic A and B_162
taagcaataaagcctcgcgtactattaccgccatcgtctggcaccatt

SEQ ID NO: 300 - DNA, 32 bp, Artificial Sequence
  Staple of surface mimic A and B_163
agagcatagcattaacatccaatagattaaga

SEQ ID NO: 301 - DNA, 40 bp, Artificial Sequence
  Staple of surface mimic A and B_164
ccccgattggtcgagggacagtacggtgtctgacaatgaa

SEQ ID NO: 302 - DNA, 48 bp, Artificial Sequence
  Staple of surface mimic A and B_165
tagagcttataaaaggtgcctgagagggagggccagaaggaaaccgag

SEQ ID NO: 303 - DNA, 40 bp, Artificial Sequence
  Staple of surface mimic A and B_166
gacggggattagaatcagagcgggatcctgagcaaattaa

SEQ ID NO: 304 - DNA, 48 bp, Artificial Sequence
  Staple of surface mimic A and B_167
agaaagcgaaaggagcaaaaaccgagatttagcggatggccagagaga

SEQ ID NO: 305 - DNA, 47 bp, Artificial Sequence
  Staple of surface mimic A and B_168
cgtgaaccataacagtatgctgtatttaacgtagagccaccaccgga

SEQ ID NO: 306 - DNA, 47 bp, Artificial Sequence
  Staple of surface mimic A and B_169
ggcgatggcccactagcgagaaaattggcagttgctggtcaaagaca

SEQ ID NO: 307 - DNA, 40 bp, Artificial Sequence
  Staple of surface mimic A and B_170
tctatcagtataaatcaaaagaatttgggcgctaaaaata

SEQ ID NO: 308 - DNA, 31 bp, Artificial Sequence
  Staple of surface mimic A and B_171
caacgtcaaagggcggggcgctagacgctca

SEQ ID NO: 309 - DNA, 48 bp, Artificial Sequence
  Staple of surface mimic A and B_172
taaagaacaaatggtcaattgctcattagacgttttcggtcatagccc

SEQ ID NO: 310 - DNA, 47 bp, Artificial Sequence
  Staple of surface mimic A and B_173
agcccgagcatttttgtttgaccaagcgctaaaccattagcaaggcc

SEQ ID NO: 311 - DNA, 40 bp, Artificial Sequence
  Staple of surface mimic A and B_174
cagggtggaattgcgttgcgctcactcacagtaagacttc

SEQ ID NO: 312 - DNA, 31 bp, Artificial Sequence
  Staple of surface mimic A and B_175
ggggtgcctaatgagcgaattcggattgcat

SEQ ID NO: 313 - DNA, 48 bp, Artificial Sequence
  Staple of surface mimic A and B_176
tcgggaaacctgtcgtcgcgtgccttcaaagctactgcggtctaagaa

SEQ ID NO: 314 - DNA, 32 bp, Artificial Sequence
  Staple of surface mimic A and B_177
tgtgtgaatatcctgactattatacaacgcta

SEQ ID NO: 315 - DNA, 40 bp, Artificial Sequence
  Staple of surface mimic A and B_178
ggtcatagctgtttccataaagtgcatcaccttttgaatg

SEQ ID NO: 316 - DNA, 36 bp, Artificial Sequence
  Staple of surface mimic A and B_179
gatcctctgtggtgctgcgggctggtaagtcaatag

SEQ ID NO: 317 - DNA, 55 bp, Artificial Sequence
  Staple of surface mimic A and B_180
cctgtgcacccgggtaccgagcttgagctaatgagagccctgatagcccggaacc

SEQ ID NO: 318 - DNA, 30 bp, Artificial Sequence
  Staple of surface mimic A and B_181
cggcggggcgtccgtgagcctcctgcccgc

SEQ ID NO: 319 - DNA, 47 bp, Artificial Sequence
  Staple of surface mimic A and B_182
cactgttgccctgcgccagaatgtcattgaaaaatatcgaagcctta

SEQ ID NO: 320 - DNA, 40 bp, Artificial Sequence
  Staple of surface mimic A and B_183
agccgggtgcaggcgctttcgcacgctgattgcaaaagaa

SEQ ID NO: 321 - DNA, 55 bp, Artificial Sequence
  Staple of surface mimic A and B_184
agcttacgggtttctttgctcgtactgcgcgttcagaaacaaaaagacacccagc

SEQ ID NO: 322 - DNA, 47 bp, Artificial Sequence
  Staple of surface mimic A and B_185
agagcacacaacactaggaataccttttcgagcaaactacaacgcct

SEQ ID NO: 323 - DNA, 32 bp, Artificial Sequence
  Staple of surface mimic A and B_186
ctcatttgaacggaacgtgccggaccaacggc

SEQ ID NO: 324 - DNA, 32 bp, Artificial Sequence
  Staple of surface mimic A and B_187
agactttctccgtggtaatttctgagatacat

SEQ ID NO: 325 - DNA, 22 bp, Artificial Sequence
  Staple of surface mimic A and B_188
gagagatgctgcaaggcgatta

SEQ ID NO: 326 - DNA, 48 bp, Artificial Sequence
  Staple of surface mimic A and B_189
taacctcaggccagtgccaagctttatcggccgcgattttaggcgcat

SEQ ID NO: 327 - DNA, 47 bp, Artificial Sequence
  Staple of surface mimic A and B_190
acgttgtagattttcaatcaaaatggaacccaaggtaaagtaattct

SEQ ID NO: 328 - DNA, 32 bp, Artificial Sequence
  Staple of surface mimic A and B_191
gcaactgtaataaggcttgccctgcaaattct

SEQ ID NO: 329 - DNA, 47 bp, Artificial Sequence
  Staple of surface mimic A and B_192
caggcaaaacgagtagccaaatcaatcgcaagcaaccatcgcccacg

SEQ ID NO: 330 - DNA, 47 bp, Artificial Sequence
  Staple of surface mimic A and B_193
taatgggatctggtgccggaaacagttgggtagtaacagttctgaat

SEQ ID NO: 331 - DNA, 47 bp, Artificial Sequence
  Staple of surface mimic A and B_194
caataggacatcaagattaattttagacagcaaccaagcgcgaaaca

SEQ ID NO: 332 - DNA, 39 bp, Artificial Sequence
  Staple of surface mimic A and B_195
agctcattttttaacattgaccgttcattactaaattgg

SEQ ID NO: 333 - DNA, 40 bp, Artificial Sequence
  Staple of surface mimic A and B_196
aaatttttatttcatttaaccttgcagaggctaaaacact

SEQ ID NO: 334 - DNA, 47 bp, Artificial Sequence
  Staple of surface mimic A and B_197
gttaaaattcgcattagatgggccatcaagattaatcattaagaata

SEQ ID NO: 335 - DNA, 40 bp, Artificial Sequence
  Staple of surface mimic A and B_198
ggagcaaatttgagagcaatagtgaatatttgtatcatcg

SEQ ID NO: 336 - DNA, 40 bp, Artificial Sequence
  Staple of surface mimic A and B_199
tagctattcaagagaaaaaacatagcaaaagaatatattc

SEQ ID NO: 337 - DNA, 32 bp, Artificial Sequence
  Staple of surface mimic A and B_200
tgataaataattgtgtcgaaatccgcgattat

SEQ ID NO: 338 - DNA, 56 bp, Artificial Sequence
  Staple of surface mimic A and B_201
gttctagctagcatgtgctattaaaaacaaaaaatgacaaacaaagaacgcgagaa

SEQ ID NO: 339 - DNA, 55 bp, Artificial Sequence
  Staple of surface mimic A and B_202
agaaaggccggagacagtcaaatatcagaaatgagtgaatgaattactaaacagc

SEQ ID NO: 340 - DNA, 32 bp, Artificial Sequence
  Staple of surface mimic A and B_203
gagatttatcataaccatcccatcgctcagta

SEQ ID NO: 341 - DNA, 32 bp, Artificial Sequence
  Staple of surface mimic A and B_204
acattattaccaaaatcaatagatgggttgat

SEQ ID NO: 342 - DNA, 24 bp, Artificial Sequence
  Staple of surface mimic A and B_205
ggaattaccaatcaattcaagaga

SEQ ID NO: 343 - DNA, 32 bp, Artificial Sequence
  Staple of surface mimic A and B_206
atcattttgaggaaggcggaacctacaagcaa

SEQ ID NO: 344 - DNA, 32 bp, Artificial Sequence
  Staple of surface mimic A and B_207
cgttattaactaacaacgtataaaaatcatta

SEQ ID NO: 345 - DNA, 32 bp, Artificial Sequence
  Staple of surface mimic A and B_208
tcgacaacataatacataacgggggatataga

SEQ ID NO: 346 - DNA, 32 bp, Artificial Sequence
  Staple of surface mimic A and B_209
ttaggagcattttaaaggggttttctaattta

SEQ ID NO: 347 - DNA, 24 bp, Artificial Sequence
  Staple of surface mimic A and B_210
acgagaatatcgtaggcagttaat

SEQ ID NO: 348 - DNA, 40 bp, Artificial Sequence
  Strand with adapter for S_A_1
tagggtcgggaatagagccgttttaatcttacgtcagaag

SEQ ID NO: 349 - DNA, 39 bp, Artificial Sequence
  Strand with adapter for S_A_2
tagggtcgggaatagaccgcgccctattttgttaagagg

SEQ ID NO: 350 - DNA, 37 bp, Artificial Sequence
  Strand with adapter for S_A_3
tagggtcgggaatagaaggcttaggttttgcgtttta

SEQ ID NO: 351 - DNA, 40 bp, Artificial Sequence
  Strand with adapter for S_A_4
aggattaggcctatttttatctaaaatcaaaaatcagacg

SEQ ID NO: 352 - DNA, 30 bp, Artificial Sequence
  Strand with adapter for S_A_5
ccaggcggcagtgccctaatagataaacag

SEQ ID NO: 353 - DNA, 28 bp, Artificial Sequence
  Strand with adapter for S_A_6
ataagtataagttttttgaggtaaatat

SEQ ID NO: 354 - DNA, 21 bp, Artificial Sequence
  Strand with adapter for S_B_1
gccccctgattagcagtttga

SEQ ID NO: 355 - DNA, 61 bp, Artificial Sequence
  Strand with adapter for S_B_2
cgagcatagaggcaacattcattgtttgtttgtgtgaagtgaagtgagtggagtgaaggta

SEQ ID NO: 356 - DNA, 42 bp, Artificial Sequence
  Staple of logic gate 2_1
aagaaaggcaaaatcccttatcatcaaccctgtttagtaaaa

SEQ ID NO: 357 - DNA, 35 bp, Artificial Sequence
  Staple of logic gate 2_2
gccgcgcgcgctggcaagtgttcggaacctcaaca

SEQ ID NO: 358 - DNA, 34 bp, Artificial Sequence
  Staple of logic gate 2_3
tttttgcgcgtaaccactttcctcgttagttttt

SEQ ID NO: 359 - DNA, 38 bp, Artificial Sequence
  Staple of logic gate 2_4
ttgcttgccgctagtaacatcataattactacaaattc

SEQ ID NO: 360 - DNA, 25 bp, Artificial Sequence
  Staple of logic gate 2_5
ctgcaagcggtcccgacagtgggat

SEQ ID NO: 361 - DNA, 35 bp, Artificial Sequence
  Staple of logic gate 2_6
cagaatctggtggttccgaaatcggcgtcaggaac

SEQ ID NO: 362 - DNA, 42 bp, Artificial Sequence
  Staple of logic gate 2_7
caggaggcacgtataacgtgccacaccctaatggttaaataa

SEQ ID NO: 363 - DNA, 34 bp, Artificial Sequence
  Staple of logic gate 2_8
tttttaatcagagcgggaattaaccgttgttttt

SEQ ID NO: 364 - DNA, 38 bp, Artificial Sequence
  Staple of logic gate 2_9
tttttatacttctttgattagtaaatatccaagagtca

SEQ ID NO: 365 - DNA, 42 bp, Artificial Sequence
  Staple of logic gate 2_10
ttttttccagtcgggaaaccaactgttgggaagggcggccag

SEQ ID NO: 366 - DNA, 32 bp, Artificial Sequence
  Staple of logic gate 2_11
cagctgcggcactgcgggcctcttcgtcacga

SEQ ID NO: 367 - DNA, 38 bp, Artificial Sequence
  Staple of logic gate 2_12
cggccttctcatggaaatacaccagtggcgattgaatt

SEQ ID NO: 368 - DNA, 38 bp, Artificial Sequence
  Staple of logic gate 2_13
tttttcgccagccattgcaacaggtaaaagggtaaatc

SEQ ID NO: 369 - DNA, 28 bp, Artificial Sequence
  Staple of logic gate 2_14
tttttagcataaagtgccaagaacctca

SEQ ID NO: 370 - DNA, 35 bp, Artificial Sequence
  Staple of logic gate 2_15
agaatggattatttccagggtattacatttaacaa

SEQ ID NO: 371 - DNA, 34 bp, Artificial Sequence
  Staple of logic gate 2_16
cacacgaaaagcgtaagaatacgtgagcagattc

SEQ ID NO: 372 - DNA, 49 bp, Artificial Sequence
  Staple of logic gate 2_17
taggcggtcagtatgatccaggggttaccaatagataatatgggaaggg

SEQ ID NO: 373 - DNA, 31 bp, Artificial Sequence
  Staple of logic gate 2_18
tttttcatcacctttgaataccaagtttttt

SEQ ID NO: 374 - DNA, 45 bp, Artificial Sequence
  Staple of logic gate 2_19
cgttaagctggtgagccgtgtccagcatcagacgtcagccggcca

SEQ ID NO: 375 - DNA, 35 bp, Artificial Sequence
  Staple of logic gate 2_20
tctttgccgccgggattgcaggcgctttcgttttt

SEQ ID NO: 376 - DNA, 39 bp, Artificial Sequence
  Staple of logic gate 2_21
aactaatttacagtcggtggtgccaatgccaatcctgat

SEQ ID NO: 377 - DNA, 42 bp, Artificial Sequence
  Staple of logic gate 2_22
aatatcttcgtattagtttgagtaacattatcaccaacaatt

SEQ ID NO: 378 - DNA, 31 bp, Artificial Sequence
  Staple of logic gate 2_23
tttttgaggaaggtgtagattttcagttttt

SEQ ID NO: 379 - DNA, 49 bp, Artificial Sequence
  Staple of logic gate 2_24
tttttcactcaatctcgtcataattttttttttacatcccttacactgg

SEQ ID NO: 380 - DNA, 35 bp, Artificial Sequence
  Staple of logic gate 2_25
gcggtataatgggtccgttttagggtaaagttaaa

SEQ ID NO: 381 - DNA, 44 bp, Artificial Sequence
  Staple of logic gate 2_26
gaggattcaaccagcttacggtggtcagttctgaatagctggca

SEQ ID NO: 382 - DNA, 42 bp, Artificial Sequence
  Staple of logic gate 2_27
attagactagattaatcaaaaaatcccgtaaaaaatgtacat

SEQ ID NO: 383 - DNA, 47 bp, Artificial Sequence
  Staple of logic gate 2_28
cgacaacttaggagaaacagaacagtaccttttacatcgaattattt

SEQ ID NO: 384 - DNA, 31 bp, Artificial Sequence
  Staple of logic gate 2_29
tttttcgttattaaaggagcggaattttttt

SEQ ID NO: 385 - DNA, 38 bp, Artificial Sequence
  Staple of logic gate 2_30
tttttaacgtgccggacttgtagacggggtccgcggtt

SEQ ID NO: 386 - DNA, 42 bp, Artificial Sequence
  Staple of logic gate 2_31
gagcacaacgcggtaaaggtttgtgttcagcaaatgtgcact

SEQ ID NO: 387 - DNA, 41 bp, Artificial Sequence
  Staple of logic gate 2_32
tgtttggctaccatgagccgtctgcagccagcggtgccgat

SEQ ID NO: 388 - DNA, 42 bp, Artificial Sequence
  Staple of logic gate 2_33
ttcatcagcacgtacactaacatctggtcagttggatcaaac

SEQ ID NO: 389 - DNA, 59 bp, Artificial Sequence
  Staple of logic gate 2_34
tttttatcatcatattcctgattaaaattgctatctaaacagttgaaaggaattttttt

SEQ ID NO: 390 - DNA, 34 bp, Artificial Sequence
  Staple of logic gate 2_35
tttttccgttccggcaatcctcataacggttttt

SEQ ID NO: 391 - DNA, 35 bp, Artificial Sequence
  Staple of logic gate 2_36
caggcggcctttagccgccagtcactgcacggtca

SEQ ID NO: 392 - DNA, 63 bp, Artificial Sequence
  Staple of logic gate 2_37
tacagtaaataaagtcagatgaagaaaccaccagattttaaaaaatcctttgcccgaattttt

SEQ ID NO: 393 - DNA, 59 bp, Artificial Sequence
  Staple of logic gate 2_38
tttttgtttaacgtcagatgaataattgcttgctgaacaatgaaaaatctaaagttttt

SEQ ID NO: 394 - DNA, 24 bp, Artificial Sequence
  Staple of logic gate 2_39
cttaaatctgtggtgctgcttttt

SEQ ID NO: 395 - DNA, 34 bp, Artificial Sequence
  Staple of logic gate 2_40
tttttgaggcgaattattcaatatatgtgttttt

SEQ ID NO: 396 - DNA, 42 bp, Artificial Sequence
  Staple of logic gate 2_41
aacggatctttcagctgcgcaggcaaagcgccattcgttttt

SEQ ID NO: 397 - DNA, 42 bp, Artificial Sequence
  Staple of logic gate 2_42
ccggaaaagatagacacagtttgttcttcgcgtccgtttttt

SEQ ID NO: 398 - DNA, 34 bp, Artificial Sequence
  Staple of logic gate 2_43
cgaaacgtacagcgccattatttcccagctatta

SEQ ID NO: 399 - DNA, 38 bp, Artificial Sequence
  Staple of logic gate 2_44
aaacagtacataaatcatttctttaatgcgcgattttt

SEQ ID NO: 400 - DNA, 34 bp, Artificial Sequence
  Staple of logic gate 2_45
tttttagtgaataacctcatagcgatagcttttt

SEQ ID NO: 401 - DNA, 56 bp, Artificial Sequence
  Staple of logic gate 2_46
tttttccattcaggaggtggagcttttttttttcgccacgggaagggatagttttt

SEQ ID NO: 402 - DNA, 38 bp, Artificial Sequence
  Staple of logic gate 2_47
agaatccttgaaaatgcttctgacattctggccttttt

SEQ ID NO: 403 - DNA, 46 bp, Artificial Sequence
  Staple of logic gate 2_48
ttcagccagctttccattaataactcacattaattgcctacgttgt

SEQ ID NO: 404 - DNA, 42 bp, Artificial Sequence
  Staple of logic gate 2_49
tttttaaccgtgcatctgccatagatgggcgcatcgtttttt

SEQ ID NO: 405 - DNA, 31 bp, Artificial Sequence
  Staple of logic gate 2_50
aggtcacgcgtctggatagggttgagttttt

SEQ ID NO: 406 - DNA, 46 bp, Artificial Sequence
  Staple of logic gate 2_51
attctccgtgagcgacagggccgaaaggagcgggccgattttacca

SEQ ID NO: 407 - DNA, 42 bp, Artificial Sequence
  Staple of logic gate 2_52
tttttaggaacgccatcaaaatcattttttaaccaatttttt

SEQ ID NO: 408 - DNA, 42 bp, Artificial Sequence
  Staple of logic gate 2_53
accgaccgtttttttttttgtgataaatttaattgagttttt

SEQ ID NO: 409 - DNA, 31 bp, Artificial Sequence
  Staple of logic gate 2_54
tttttacaacgccacaccaccctcatttttt

SEQ ID NO: 410 - DNA, 27 bp, Artificial Sequence
  Staple of logic gate 2_55
cctgagagtctggcccagctaaatatc

SEQ ID NO: 411 - DNA, 42 bp, Artificial Sequence
  Staple of logic gate 2_56
ttctagctgttttttttttataaattaaataatcagattttt

SEQ ID NO: 412 - DNA, 31 bp, Artificial Sequence
  Staple of logic gate 2_57
tttttgtaattctgtcaccgtactcattttt

SEQ ID NO: 413 - DNA, 49 bp, Artificial Sequence
  Staple of logic gate 2_58
tagaacccacgcatagcttgataccgataaaggagatgatatgagggta

SEQ ID NO: 414 - DNA, 49 bp, Artificial Sequence
  Staple of logic gate 2_59
aacgcaaggtcgcttcgaggtgaatttccggtttaacagtcagatctac

SEQ ID NO: 415 - DNA, 34 bp, Artificial Sequence
  Staple of logic gate 2_60
tacttttgcgggagttaaaggggttttgctcagt

SEQ ID NO: 416 - DNA, 31 bp, Artificial Sequence
  Staple of logic gate 2_61
tttttatcggctgtctgaaacatgaattttt

SEQ ID NO: 417 - DNA, 48 bp, Artificial Sequence
  Staple of logic gate 2_62
ttaacattagctatgaacgaggcgcattttttttttaggcaaaagaat

SEQ ID NO: 418 - DNA, 38 bp, Artificial Sequence
  Staple of logic gate 2_63
ttctaccgcgagtgctcctgaccaactttgaaagagca

SEQ ID NO: 419 - DNA, 45 bp, Artificial Sequence
  Staple of logic gate 2_64
cggtatagcctttttgtaccaccggaaccgctagccccagttaca

SEQ ID NO: 420 - DNA, 56 bp, Artificial Sequence
  Staple of logic gate 2_65
tttttttaccgcgcaattttatcttttttttttctgaatctttccaaataattttt

SEQ ID NO: 421 - DNA, 34 bp, Artificial Sequence
  Staple of logic gate 2_66
tttttcaataacctgttccaataaatcatttttt

SEQ ID NO: 422 - DNA, 35 bp, Artificial Sequence
  Staple of logic gate 2_67
cagctacccaatagttcatcgtaggaatcattttt

SEQ ID NO: 423 - DNA, 47 bp, Artificial Sequence
  Staple of logic gate 2_68
cctaatttgcattccatatagttttgatctaagaaccaaaaacatct

SEQ ID NO: 424 - DNA, 34 bp, Artificial Sequence
  Staple of logic gate 2_69
ttttttagagcttaattttcgcaaatggtttttt

SEQ ID NO: 425 - DNA, 35 bp, Artificial Sequence
  Staple of logic gate 2_70
tgtagcttagtttgtttggggtaatagtttaagca

SEQ ID NO: 426 - DNA, 59 bp, Artificial Sequence
  Staple of logic gate 2_71
tttttgagcttcaaagcgaaccagccttttggcgatttctttaatcattgtgaattttt

SEQ ID NO: 427 - DNA, 35 bp, Artificial Sequence
  Staple of logic gate 2_72
ctgaacatagacggacttgagagaacgagtagtaa

SEQ ID NO: 428 - DNA, 35 bp, Artificial Sequence
  Staple of logic gate 2_73
aagtcaggcaaagctttaccccttattacgcagta

SEQ ID NO: 429 - DNA, 42 bp, Artificial Sequence
  Staple of logic gate 2_74
gataaccagcctttagcaccagaaaccatcgatagcagcagc

SEQ ID NO: 430 - DNA, 49 bp, Artificial Sequence
  Staple of logic gate 2_75
cacaagacaatgaagccgaactaataacggaatacacgcaaacaataga

SEQ ID NO: 431 - DNA, 56 bp, Artificial Sequence
  Staple of logic gate 2_76
agacttcagttcaggaatcgtcataaatagttttgaatagcgagaggctttttttt

SEQ ID NO: 432 - DNA, 38 bp, Artificial Sequence
  Staple of logic gate 2_77
gtcagaaagggtaattgagcataaaagagccattgccc

SEQ ID NO: 433 - DNA, 42 bp, Artificial Sequence
  Staple of logic gate 2_78
ttttttaataagagcaagaaaattgagttaagcccaattttt

SEQ ID NO: 434 - DNA, 59 bp, Artificial Sequence
  Staple of logic gate 2_79
tttttcccctcaaatgctttaaacaaatatcgaaagattaacggaacaacattattttt

SEQ ID NO: 435 - DNA, 35 bp, Artificial Sequence
  Staple of logic gate 2_80
gcagataatagcaaaaagacaaggtaaatattgac

SEQ ID NO: 436 - DNA, 21 bp, Artificial Sequence
  Staple of logic gate 2_81
tgttacttcaaaaatcaggtc

SEQ ID NO: 437 - DNA, 27 bp, Artificial Sequence
  Staple of logic gate 2_82
aagactctgactattacctttttaaga

SEQ ID NO: 438 - DNA, 42 bp, Artificial Sequence
  Staple of logic gate 2_83
tttttaaccgaggaaacgcaaaaagttaccagaaggattttt

SEQ ID NO: 439 - DNA, 31 bp, Artificial Sequence
  Staple of logic gate 2_84
ttttttgcaaaagaattcattgaatcttttt

SEQ ID NO: 440 - DNA, 56 bp, Artificial Sequence
  Staple of logic gate 2_85
atgtttagagcaaacgtagaaactatcataaccctattcaacatcaaaagaacacc

SEQ ID NO: 441 - DNA, 42 bp, Artificial Sequence
  Staple of logic gate 2_86
aaattcatatggtaaacatataaaagaaccaaaagaaagtaa

SEQ ID NO: 442 - DNA, 42 bp, Artificial Sequence
  Staple of logic gate 2_87
ttttttttattttggacattcaattttttttttccgattgag

SEQ ID NO: 443 - DNA, 31 bp, Artificial Sequence
  Staple of logic gate 2_88
tttttttacaggtagcgttttaattcttttt

SEQ ID NO: 444 - DNA, 42 bp, Artificial Sequence
  Staple of logic gate 2_89
tcatcagaaaccaaccagagggggtaattactgcgaaaacga

SEQ ID NO: 445 - DNA, 41 bp, Artificial Sequence
  Staple of logic gate 2_90
ttgagatgcccgaagcaaactccaacaggtcactagattaa

SEQ ID NO: 446 - DNA, 35 bp, Artificial Sequence
  Staple of logic gate 2_91
gaattatgatacatgagcaacaatacatacataaa

SEQ ID NO: 447 - DNA, 31 bp, Artificial Sequence
  Staple of logic gate 2_92
tttttttaccttatataagaggtcatttttt

SEQ ID NO: 448 - DNA, 42 bp, Artificial Sequence
  Staple of logic gate 2_93
gcaaaatggaaattattttaccagcgcctagctattcagaga

SEQ ID NO: 449 - DNA, 49 bp, Artificial Sequence
  Staple of logic gate 2_94
ttaccatggagggaaaagggctcacaatgacaccacggaataagttttt

SEQ ID NO: 450 - DNA, 55 bp, Artificial Sequence
  Staple of logic gate 2_95
ttcagtgaatcagtagcgactcggtcactccctcccaccagaaccagaaaatcaa

SEQ ID NO: 451 - DNA, 34 bp, Artificial Sequence
  Staple of logic gate 2_96
tttttactgtagcgcgtcagaaccgccacttttt

SEQ ID NO: 452 - DNA, 34 bp, Artificial Sequence
  Staple of logic gate 2_97
tttttcctcagagccacaggaggttgaggttttt

SEQ ID NO: 453 - DNA, 59 bp, Artificial Sequence
  Staple of logic gate 2_98
acactaattagccgattttcaaccattagatacatgctgaatggctggctgaccttttt

SEQ ID NO: 454 - DNA, 34 bp, Artificial Sequence
  Staple of logic gate 2_99
gaccccccgcgaccctgaaaagaacgagtagatt

SEQ ID NO: 455 - DNA, 35 bp, Artificial Sequence
  Staple of logic gate 2_100
gaaagcgcaaaaatacgtaatcctcagagcatcaa

SEQ ID NO: 456 - DNA, 42 bp, Artificial Sequence
  Staple of logic gate 2_101
tgatattccgccagcattgaccaccctcttgcaccctaacga

SEQ ID NO: 457 - DNA, 34 bp, Artificial Sequence
  Staple of logic gate 2_102
tttttcaggtcagacgagtactggtaatattttt

SEQ ID NO: 458 - DNA, 45 bp, Artificial Sequence
  Staple of logic gate 2_103
tttttgaggactaaagactttttccaccaacagcaaaaagtagca

SEQ ID NO: 459 - DNA, 42 bp, Artificial Sequence
  Staple of logic gate 2_104
caggagtttggcctttttattcaagcaaatcagattgctatt

SEQ ID NO: 460 - DNA, 42 bp, Artificial Sequence
  Staple of logic gate 2_105
gggtagccatcgccctcatattaaagattcaaaagcatcaat

SEQ ID NO: 461 - DNA, 28 bp, Artificial Sequence
  Staple of logic gate 2_106
cagcagcgaaagactatattcggataaa

SEQ ID NO: 462 - DNA, 59 bp, Artificial Sequence
  Staple of logic gate 2_107
ttttttaacggggtcagtgccttgtattattctttccttagaaaccaatcaatattttt

SEQ ID NO: 463 - DNA, 34 bp, Artificial Sequence
  Staple of logic gate 2_108
tttttaatgacaacaacaacggctacagattttt

SEQ ID NO: 464 - DNA, 48 bp, Artificial Sequence
  Staple of logic gate 2_109
gcaatgccccctgcgggtatccatcctaatttacataaacagtatagc

SEQ ID NO: 465 - DNA, 34 bp, Artificial Sequence
  Staple of logic gate 2_110
tttttaaaaaggctccaagttgcgccgacttttt

SEQ ID NO: 466 - DNA, 21 bp, Artificial Sequence
  Staple of logic gate 2_111
agatgcccgcagtgcagttgg

SEQ ID NO: 467 - DNA, 28 bp, Artificial Sequence
  Staple of logic gate 2_112
tcaatcgtctgactgaatcggggcgcca

SEQ ID NO: 468 - DNA, 30 bp, Artificial Sequence
  Staple of logic gate 2_113
ggaaggggaaagcctcatatgcgttataga

SEQ ID NO: 469 - DNA, 21 bp, Artificial Sequence
  Staple of logic gate 2_114
tagaaagggattttgaatcga

SEQ ID NO: 470 - DNA, 28 bp, Artificial Sequence
  Staple of logic gate 2_115
gcgcgaactgataagcgggagacagttg

SEQ ID NO: 471 - DNA, 28 bp, Artificial Sequence
  Staple of logic gate 2_116
actgggaagaaaaaataccaccgtttac

SEQ ID NO: 472 - DNA, 28 bp, Artificial Sequence
  Staple of logic gate 2_117
cggattgaccgtaatatcggcctcaggg

SEQ ID NO: 473 - DNA, 33 bp, Artificial Sequence
  Staple of logic gate 2_118
gaaaaatatgcagacgtcgagagggttttagcg

SEQ ID NO: 474 - DNA, 38 bp, Artificial Sequence
  Staple of logic gate 2_119
gcctcgtggtgctggtcctggaggggtcactgtcattt

SEQ ID NO: 475 - DNA, 33 bp, Artificial Sequence
  Staple of logic gate 2_120
atataacggcagcattttgcggaacaatggcaa

SEQ ID NO: 476 - DNA, 39 bp, Artificial Sequence
  Staple of logic gate 2_121
ccataaaggatagcgtccaaagtaaacagacgacgataa

SEQ ID NO: 477 - DNA, 39 bp, Artificial Sequence
  Staple of logic gate 2_122
aactggcatgattggtggccgaggcatagtaaaacgcca

SEQ ID NO: 478 - DNA, 26 bp, Artificial Sequence
  Staple of logic gate 2_123
aaaagattaaatgtgggaaatataac

SEQ ID NO: 479 - DNA, 40 bp, Artificial Sequence
  Staple of logic gate 2_124
ctgtatggaacaacgcgcggataagtgcacgcgcccattg

SEQ ID NO: 480 - DNA, 33 bp, Artificial Sequence
  Staple of logic gate 2_125
gtgtccactattataaacgttaatattttgtta

SEQ ID NO: 481 - DNA, 26 bp, Artificial Sequence
  Staple of logic gate 2_126
ctacattgaactcaaactataaaatc

SEQ ID NO: 482 - DNA, 28 bp, Artificial Sequence
  Staple of logic gate 2_127
tgtaaagcaacatacgagccggattttt

SEQ ID NO: 483 - DNA, 60 bp, Artificial Sequence
  Staple of logic gate 2_128
atgagtgtatccgctcacaacccgggcggaatttgtgagcaatcggaaaacgacgatcgg

SEQ ID NO: 484 - DNA, 35 bp, Artificial Sequence
  Staple of logic gate 2_129
acattggctgtttcctgtgtgtcgtaataaacaaa

SEQ ID NO: 485 - DNA, 66 bp, Artificial Sequence
  Staple of logic gate 2_130
tttttaacagagatagaacccttcattagtcaattacctgagcaattaatgggtcgctattaataa

SEQ ID NO: 486 - DNA, 28 bp, Artificial Sequence
  Staple of logic gate 2_131
tttttgagcctcctctttctccgtggtg

SEQ ID NO: 487 - DNA, 26 bp, Artificial Sequence
  Staple of logic gate 2_132
gacaattgatgaaacaaactttcatt

SEQ ID NO: 488 - DNA, 47 bp, Artificial Sequence
  Staple of logic gate 2_133
cagtgccccctgccgacataaaggagaaacaatcctcaatccacgct

SEQ ID NO: 489 - DNA, 45 bp, Artificial Sequence
  Staple of logic gate 2_134
tttttagacgcagaaacagcggatcaaacgatgctgattgttttt

SEQ ID NO: 490 - DNA, 19 bp, Artificial Sequence
  Staple of logic gate 2_135
ttctgctcatttgtgatga

SEQ ID NO: 491 - DNA, 56 bp, Artificial Sequence
  Staple of logic gate 2_136
gcggttgagccgcattagaacattataccagcaaccgcaagatcccacgtagaagt

SEQ ID NO: 492 - DNA, 15 bp, Artificial Sequence
  Staple of logic gate 2_137
aacggattcgcctgt

SEQ ID NO: 493 - DNA, 49 bp, Artificial Sequence
  Staple of logic gate 2_138
atcaagacatggtccagcagaagataaaacagaggctcgaataaattgt

SEQ ID NO: 494 - DNA, 21 bp, Artificial Sequence
  Staple of logic gate 2_139
taagttgagggggaataggtc

SEQ ID NO: 495 - DNA, 34 bp, Artificial Sequence
  Staple of logic gate 2_140
tgtgctgctaattttcccttatagtgaatttatc

SEQ ID NO: 496 - DNA, 42 bp, Artificial Sequence
  Staple of logic gate 2_141
cgcttctggtgccggaaacctcgctcactgcccgcttttttt

SEQ ID NO: 497 - DNA, 41 bp, Artificial Sequence
  Staple of logic gate 2_142
tttttttagattaagacgctgagagaacaatattacttttt

SEQ ID NO: 498 - DNA, 27 bp, Artificial Sequence
  Staple of logic gate 2_143
gggacgaacgctggcaggcgaaaatcc

SEQ ID NO: 499 - DNA, 21 bp, Artificial Sequence
  Staple of logic gate 2_144
tatatgtttttagaactatgg

SEQ ID NO: 500 - DNA, 28 bp, Artificial Sequence
  Staple of logic gate 2_145
taatttcatcttcctgatgcaaatccaa

SEQ ID NO: 501 - DNA, 56 bp, Artificial Sequence
  Staple of logic gate 2_146
tttttggaagattgtataagcaaatattaatcagcataattcgttggtggtttgag

SEQ ID NO: 502 - DNA, 28 bp, Artificial Sequence
  Staple of logic gate 2_147
gtataaacaccggaaactgacctaaatt

SEQ ID NO: 503 - DNA, 63 bp, Artificial Sequence
  Staple of logic gate 2_148
gtagggcaaggcgtttgaaataaatataacaaagaacgcgagaattttttttttaactttttc

SEQ ID NO: 504 - DNA, 42 bp, Artificial Sequence
  Staple of logic gate 2_149
tgaacggatcaggttgtttatcaacaagtcagctttaaagga

SEQ ID NO: 505 - DNA, 35 bp, Artificial Sequence
  Staple of logic gate 2_150
atttaggataaagtgacgacagagcatgtatcatt

SEQ ID NO: 506 - DNA, 35 bp, Artificial Sequence
  Staple of logic gate 2_151
agtaataagagaatcagaggccagaaccacccatg

SEQ ID NO: 507 - DNA, 28 bp, Artificial Sequence
  Staple of logic gate 2_152
aaaggccggatagataagtccttatttc

SEQ ID NO: 508 - DNA, 35 bp, Artificial Sequence
  Staple of logic gate 2_153
attttaaatgcaatgcttttttttttctgagtaat

SEQ ID NO: 509 - DNA, 42 bp, Artificial Sequence
  Staple of logic gate 2_154
ccaagaacctatttgagaaggattaggagatataaacatgtt

SEQ ID NO: 510 - DNA, 41 bp, Artificial Sequence
  Staple of logic gate 2_155
tttttacaggcaaggcaaagaattctaaaacgaaagttttt

SEQ ID NO: 511 - DNA, 18 bp, Artificial Sequence
  Staple of logic gate 2_156
ataaaggccactatcttt

SEQ ID NO: 512 - DNA, 42 bp, Artificial Sequence
  Staple of logic gate 2_157
aaataaatccagaggattagtatagaagaacaagcaagccgt

SEQ ID NO: 513 - DNA, 15 bp, Artificial Sequence
  Staple of logic gate 2_158
gagaattaaggatta

SEQ ID NO: 514 - DNA, 28 bp, Artificial Sequence
  Staple of logic gate 2_159
tttttttaacgtcaaaaatgaaaatagc

SEQ ID NO: 515 - DNA, 59 bp, Artificial Sequence
  Staple of logic gate 2_160
gaatgagaggaattaggatctacgttaataatggctcatacctttaattgctaccggaa

SEQ ID NO: 516 - DNA, 28 bp, Artificial Sequence
  Staple of logic gate 2_161
taaaggtggaattaacagggaagcgcat

SEQ ID NO: 517 - DNA, 53 bp, Artificial Sequence
  Staple of logic gate 2_162
caccagtacagagagaataacgctaatacttaccgaagctaaaaggaattcat

SEQ ID NO: 518 - DNA, 56 bp, Artificial Sequence
  Staple of logic gate 2_163
acccaaaatgacagatgaacggttttaaatatgcaattctgcggacctcccgactt

SEQ ID NO: 519 - DNA, 38 bp, Artificial Sequence
  Staple of logic gate 2_164
tttttgacggtcaatcataagggagcgcataataatgc

SEQ ID NO: 520 - DNA, 54 bp, Artificial Sequence
  Staple of logic gate 2_165
attgtgtaaatcaccggaaccgccatcttacggtgtctggaagtttcccttatt

SEQ ID NO: 521 - DNA, 49 bp, Artificial Sequence
  Staple of logic gate 2_166
agagccgagagccgccacccttttcatcattatttatcccaaaccaacg

SEQ ID NO: 522 - DNA, 56 bp, Artificial Sequence
  Staple of logic gate 2_167
tttaccgcagaatgcggttgtacgcgaggcgttttagcgatggcataaagctaaat

SEQ ID NO: 523 - DNA, 63 bp, Artificial Sequence
  Staple of logic gate 2_168
cctttaaattttttcacgttgttgtcgtatgtaccccggttgtgccggatcaaccggtgtagg

SEQ ID NO: 524 - DNA, 51 bp, Artificial Sequence
  Staple of logic gate 2_169
attgcgaagtaaataaaactagcatgtttgagaaatcacggtgagaatttt

SEQ ID NO: 525 - DNA, 38 bp, Artificial Sequence
  Staple of logic gate 2_170
tttttggaggtttagtaccgccactcagagcacatgta

SEQ ID NO: 526 - DNA, 53 bp, Artificial Sequence
  Strand with antiCD19 adapter sequence for switchable variant_1
ttcgtctcgtcatccctgcgcggcatcgcagtaaagttcaatagggtcaaagc

SEQ ID NO: 527 - DNA, 47 bp, Artificial Sequence
  Strand with antiCD19 adapter sequence for switchable variant_2
tgagagagcggggattgacgcgcagtaaagttcaatagggtcaaagc

SEQ ID NO: 528 - DNA, 61 bp, Artificial Sequence
  Strand with antiCD19 adapter sequence for switchable variant_3
ttcgcatctgtagccagctttaaatcaacgagaaagcagtaaagttcaatagggtcaaagc

SEQ ID NO: 529 - DNA, 47 bp, Artificial Sequence
  Strand with antiCD19 adapter sequence for switchable variant_4
ggattgctaatgcacaccgtcgcagtaaagttcaatagggtcaaagc

SEQ ID NO: 530 - DNA, 47 bp, Artificial Sequence
  Strand with antiCD19 adapter sequence for switchable variant_5
cgaaatcagcgattataccaagcagtaaagttcaatagggtcaaagc

SEQ ID NO: 531 - DNA, 53 bp, Artificial Sequence
  Strand with antiCD19 adapter sequence for switchable variant_6
accgccaccgtttcagcggagtgagaagcagtaaagttcaatagggtcaaagc

SEQ ID NO: 532 - DNA, 34 bp, Artificial Sequence
  Strand with antiCD19 adapter sequence for switchable variant_7
ttttttaaagcactaaaagcggtcacgctttttt

SEQ ID NO: 533 - DNA, 44 bp, Artificial Sequence
  Strand with antiCD19 adapter sequence for switchable variant_8
ttttttgttgttccagtttggaacaagaggaagaatatgtttga

SEQ ID NO: 534 - DNA, 59 bp, Artificial Sequence
  Strand with antiCD19 adapter sequence for switchable variant_9
tttttagtattaagaggctgagacaggtgtatccagacaccgacaaaaggtaaattttt

SEQ ID NO: 535 - DNA, 34 bp, Artificial Sequence
  Strand with antiCD19 adapter sequence for switchable variant_10
tttttcgatctaaagttaaaatctccaaattttt

SEQ ID NO: 536 - DNA, 60 bp, Artificial Sequence
  Strand with antiCD19 adapter sequence for non-switchable variant_1
ttttttaaagcactaaaagcggtcacgcttttttgcagtaaagttcaatagggtcaaagc

SEQ ID NO: 537 - DNA, 70 bp, Artificial Sequence
  Strand with antiCD19 adapter sequence for non-switchable variant_2
gcagtaaagttcaatagggtcaaagcttttttgttgttccagtttggaacaagaggaagaatatgtttga

SEQ ID NO: 538 - DNA, 85 bp, Artificial Sequence
  Strand with antiCD19 adapter sequence for non-switchable variant_3
gcagtaaagttcaatagggtcaaagctttttagtattaagaggctgagacaggtgtatccagacaccgacaaaaggtaaattttt

SEQ ID NO: 539 - DNA, 60 bp, Artificial Sequence
  Strand with antiCD19 adapter sequence for non-switchable variant_4
tttttcgatctaaagttaaaatctccaaatttttgcagtaaagttcaatagggtcaaagc

SEQ ID NO: 540 - DNA, 61 bp, Artificial Sequence; identical to SEQ ID NO: 528
  Strand with antiCD19 adapter sequence for non-switchable variant_5

SEQ ID NO: 541 - DNA, 53 bp, Artificial Sequence; identical to SEQ ID NO: 531
  Strand with antiCD19 adapter sequence for non-switchable variant_6

SEQ ID NO: 542 - DNA, 87 bp, Artificial Sequence
  Strand with antiCD3 adapter strand_1
gaagtgtttttataggtacgcttgggttcaaacgggcgagagtggagatggtgagtaaggcgcgagagtggagatggtgagtaaggc

SEQ ID NO: 543 - DNA, 87 bp, Artificial Sequence
  Strand with antiCD3 adapter strand_2
cttttgcgggatcggcagggagaagccttgaacaagcgagagtggagatggtgagtaaggcgcgagagtggagatggtgagtaaggc

SEQ ID NO: 544 - DNA, 47 bp, Artificial Sequence
  T0 hinge molecule for logic gate 2_1
ttttttccaacgtcaaagggcgacattccgacgttataatattgtat

SEQ ID NO: 545 - DNA, 28 bp, Artificial Sequence
  T0 hinge molecule for logic gate 2_2
ggcgaactctatcagggcgattacaaac

SEQ ID NO: 546 - DNA, 39 bp, Artificial Sequence
  T0 hinge molecule for logic gate 2_3
tagagcttacgtgaaccatcacactgaactttcaacact

SEQ ID NO: 547 - DNA, 49 bp, Artificial Sequence
  T0 hinge molecule for logic gate 2_4
gcccgagccttctaaatttttgttataaattgaagaacgtggacttttt

SEQ ID NO: 548 - DNA, 43 bp, Artificial Sequence
  T0 hinge molecule for logic gate 2_5
tttttcccagtttgcaccgcctagtgagacgggcaacattttt

SEQ ID NO: 549 - DNA, 27 bp, Artificial Sequence
  T0 hinge molecule for logic gate 2_6
ccgattaaaagagtacttgcctgagta

SEQ ID NO: 550 - DNA, 26 bp, Artificial Sequence
  T0 hinge molecule for logic gate 2_7
agagagttgcagagggtggtttttct

SEQ ID NO: 551 - DNA, 56 bp, Artificial Sequence
  T0 hinge molecule for logic gate 2_8
atcagtgcggtttgcgtattgccaacgcctaccttcgccagctggcgaaggtaacg

SEQ ID NO: 552 - DNA, 38 bp, Artificial Sequence
  T0 hinge molecule for logic gate 2_9
gaagaggaggccaccgagtaaagggaaaatgccgtcgg

SEQ ID NO: 553 - DNA, 49 bp, Artificial Sequence
  T0 hinge molecule for logic gate 2_10
gctggtataacatcctgtccatcacgcaagctaaatcgcaagttttagt

SEQ ID NO: 554 - DNA, 35 bp, Artificial Sequence
  T0 hinge molecule for logic gate 2_11
cctggggtgcgttggtcgtgctttcaccggccctg

SEQ ID NO: 555 - DNA, 45 bp, Artificial Sequence
  T0 hinge molecule for logic gate 2_12
tttttactgatagccctaaaacatagcagcactcaaatcaaatca

SEQ ID NO: 556 - DNA, 45 bp, Artificial Sequence
  T0 hinge molecule for logic gate 2_13
tttttggccagaatgcggcgggccgcgtgccgaggatcttccaca

SEQ ID NO: 557 - DNA, 42 bp, Artificial Sequence
  T0 hinge molecule for logic gate 2_14
gagagcccgccattaatggcttgacctgccagtaaaaaaacg

SEQ ID NO: 558 - DNA, 35 bp, Artificial Sequence
  T0 hinge molecule for logic gate 2_15
atcagactaacaccgcctgcagaaccacatgcaca

SEQ ID NO: 559 - DNA, 46 bp, Artificial Sequence
  T0 hinge molecule for logic gate 2_16
atgtttaccagtcctaccgagtttctgccagcacgttttcgcgcct

SEQ ID NO: 560 - DNA, 40 bp, Artificial Sequence
  T0 hinge molecule for logic gate 2_17
accttttaagaagaatttttgaaaaataccgaacacagtg

SEQ ID NO: 561 - DNA, 49 bp, Artificial Sequence
  T0 hinge molecule for logic gate 2_18
gaataaagccaacgcctaaagtcaagttttttgggaataggagccaccc

SEQ ID NO: 562 - DNA, 48 bp, Artificial Sequence
  T0 hinge molecule for logic gate 2_19
agagcaaacaagagctaaacagtttcgtcaccagggcccactgacggg

SEQ ID NO: 563 - DNA, 52 bp, Artificial Sequence
  T0 hinge molecule for logic gate 2_20
gctatttcaatcatctttccaacagacagccctcatagttagcgtaattttt

SEQ ID NO: 564 - DNA, 42 bp, Artificial Sequence
  T0 hinge molecule for logic gate 2_21
aaaggcttaatcgtgaatttttacaacgcctgtagaaaaccg

SEQ ID NO: 565 - DNA, 49 bp, Artificial Sequence
  T0 hinge molecule for logic gate 2_22
attcccaactaaagtttcatatcaacgtaacaaagaaacaccccatttg

SEQ ID NO: 566 - DNA, 42 bp, Artificial Sequence
  T0 hinge molecule for logic gate 2_23
gcgtcttcagccatggcatttagaatcaagtttgccaccaat

SEQ ID NO: 567 - DNA, 46 bp, Artificial Sequence
  T0 hinge molecule for logic gate 2_24
caacatgtgtacccggatattcattattgggccagtcaggacgtcg

SEQ ID NO: 568 - DNA, 45 bp, Artificial Sequence
  T0 hinge molecule for logic gate 2_25
gagagttatacttgagatggtttaacaagaaagaccagaccgaac

SEQ ID NO: 569 - DNA, 45 bp, Artificial Sequence
  T0 hinge molecule for logic gate 2_26
tagcaaggcttttttttttcggaaacgtctttagcgtcagttttt

SEQ ID NO: 570 - DNA, 56 bp, Artificial Sequence
  T0 hinge molecule for logic gate 2_27
tgacgagctgctcaagcgtttagagccatcatcgcacaaagtttaaagcttccagt

SEQ ID NO: 571 - DNA, 45 bp, Artificial Sequence
  T0 hinge molecule for logic gate 2_28
tttttttcatcaagagtaatcttgatttcaataagaacaacgaac

SEQ ID NO: 572 - DNA, 35 bp, Artificial Sequence
  T0 hinge molecule for logic gate 2_29
atgttacaacactcacgaaggatgaggaggaacga

SEQ ID NO: 573 - DNA, 47 bp, Artificial Sequence
  T0 hinge molecule for logic gate 2_30
tcctcaacaacggaccaccagagccgcacaaacggcttttgtgcccg

SEQ ID NO: 574 - DNA, 47 bp, Artificial Sequence
  T0 hinge molecule for logic gate 2_31
tataaacagttcgaagcgtcatacataaataaacatcgaggcttatc

SEQ ID NO: 575 - DNA, 38 bp, Artificial Sequence
  T0 hinge molecule for logic gate 2_32
ttaaacaaccgaagcatcagtttccattaaacgggtgt

SEQ ID NO: 576 - DNA, 28 bp, Artificial Sequence
  T0 hinge molecule for logic gate 2_33
accagttgaggctttcaccctctctgaa

SEQ ID NO: 577 - DNA, 42 bp, Artificial Sequence
  T0 hinge molecule for logic gate 2_34
cctcagaccggaattcctcaacggaaccagtaacagatgata

SEQ ID NO: 578 - DNA, 42 bp, Artificial Sequence
  T0 hinge molecule for logic gate 2_35
taccgtaacccaaaggagccccgctaggttaatgctgacgag

SEQ ID NO: 579 - DNA, 41 bp, Artificial Sequence
  T0 hinge molecule for logic gate 2_36
ttttttttcagggatagcaagcccgtcgaggtgccgttttt

SEQ ID NO: 580 - DNA, 49 bp, Artificial Sequence
  T2 hinge molecule for logic gate 2_1
ttttttccaacgtcaaagggcgattcattccgacgttataatattgtat

SEQ ID NO: 581 - DNA, 30 bp, Artificial Sequence
  T2 hinge molecule for logic gate 2_2
ggcgaactctatcagggcgattttacaaac

SEQ ID NO: 582 - DNA, 41 bp, Artificial Sequence
  T2 hinge molecule for logic gate 2_3
tagagcttacgtgaaccatcttacactgaactttcaacact

SEQ ID NO: 583 - DNA, 49 bp, Artificial Sequence; identical to SEQ ID NO: 547
  T2 hinge molecule for logic gate 2_4

SEQ ID NO: 584 - DNA, 47 bp, Artificial Sequence
  T2 hinge molecule for logic gate 2_5
tttttcccagtttgcttaccgcctttagtgagacgggcaacattttt

SEQ ID NO: 585 - DNA, 29 bp, Artificial Sequence
  T2 hinge molecule for logic gate 2_6
ccgattaaaagagtttacttgcctgagta

SEQ ID NO: 586 - DNA, 28 bp, Artificial Sequence
  T2 hinge molecule for logic gate 2_7
agagagttgcagattgggtggtttttct

SEQ ID NO: 587 - DNA, 58 bp, Artificial Sequence
  T2 hinge molecule for logic gate 2_8
atcagtgttcggtttgcgtattgccaacgcctaccttcgccagctggcgaaggtaacg

SEQ ID NO: 588 - DNA, 40 bp, Artificial Sequence
  T2 hinge molecule for logic gate 2_9
gaagaggttaggccaccgagtaaagggaaaatgccgtcgg

SEQ ID NO: 589 - DNA, 51 bp, Artificial Sequence
  T2 hinge molecule for logic gate 2_10
gctggtataacatcttctgtccatcacgcaagctaaatcgcaagttttagt

SEQ ID NO: 590 - DNA, 37 bp, Artificial Sequence
  T2 hinge molecule for logic gate 2_11
cctggggtgcgttggtcgtgctttcaccttggccctg

SEQ ID NO: 591 - DNA, 47 bp, Artificial Sequence
  T2 hinge molecule for logic gate 2_12
tttttactgatagccctaaaacatttagcagcactcaaatcaaatca

SEQ ID NO: 592 - DNA, 47 bp, Artificial Sequence
  T2 hinge molecule for logic gate 2_13
tttttggccagaatgcggcgggccttgcgtgccgaggatcttccaca

SEQ ID NO: 593 - DNA, 44 bp, Artificial Sequence
  T2 hinge molecule for logic gate 2_14
gagagccttcgccattaatggcttgacctgccagtaaaaaaacg

SEQ ID NO: 594 - DNA, 37 bp, Artificial Sequence
  T2 hinge molecule for logic gate 2_15
atcagactaacaccgcctgcattgaaccacatgcaca

SEQ ID NO: 595 - DNA, 48 bp, Artificial Sequence
  T2 hinge molecule for logic gate 2_16
atgtttaccagtcctaccgagtttctgccagcacttgttttcgcgcct

SEQ ID NO: 596 - DNA, 42 bp, Artificial Sequence
  T2 hinge molecule for logic gate 2_17
accttttaagaagaatttttgaaaaataccgaacttacagtg

SEQ ID NO: 597 - DNA, 51 bp, Artificial Sequence
  T2 hinge molecule for logic gate 2_18
gaataaagccaacgcctaaagtcaagttttttgggttaataggagccaccc

SEQ ID NO: 598 - DNA, 50 bp, Artificial Sequence
  T2 hinge molecule for logic gate 2_19
agagcaaacaagagctaaacagtttcgtcaccagttggcccactgacggg

SEQ ID NO: 599 - DNA, 54 bp, Artificial Sequence
  T2 hinge molecule for logic gate 2_20
gctatttcaatcatctttccaacagacagccctcatagttagcgtaattttttt

SEQ ID NO: 600 - DNA, 44 bp, Artificial Sequence
  T2 hinge molecule for logic gate 2_21
aaaggcttaatcgtgaatttttacaacgcctgtagttaaaaccg

SEQ ID NO: 601 - DNA, 51 bp, Artificial Sequence
  T2 hinge molecule for logic gate 2_22
attcccaactaaagtttcatatcaacgtaacaaagttaaacaccccatttg

SEQ ID NO: 602 - DNA, 44 bp, Artificial Sequence
  T2 hinge molecule for logic gate 2_23
gcgtcttcagccatggcatttagaatcaagtttgcttcaccaat

SEQ ID NO: 603 - DNA, 48 bp, Artificial Sequence
  T2 hinge molecule for logic gate 2_24
caacatgtgtacccggatattcattttattgggccagtcaggacgtcg

SEQ ID NO: 604 - DNA, 47 bp, Artificial Sequence
  T2 hinge molecule for logic gate 2_25
gagagttatacttgagatggtttattacaagaaagaccagaccgaac

SEQ ID NO: 605 - DNA, 47 bp, Artificial Sequence
  T2 hinge molecule for logic gate 2_26
tagcaaggcttttttttttcggaaacgtttctttagcgtcagttttt

SEQ ID NO: 606 - DNA, 58 bp, Artificial Sequence
  T2 hinge molecule for logic gate 2_27
tgacgagttctgctcaagcgtttagagccatcatcgcacaaagtttaaagcttccagt

SEQ ID NO: 607 - DNA, 47 bp, Artificial Sequence
  T2 hinge molecule for logic gate 2_28
tttttttcatcaagagtaatcttgttatttcaataagaacaacgaac

SEQ ID NO: 608 - DNA, 37 bp, Artificial Sequence
  T2 hinge molecule for logic gate 2_29
atgttacaacactcacgaaggatgaggattggaacga

SEQ ID NO: 609 - DNA, 49 bp, Artificial Sequence
  T2 hinge molecule for logic gate 2_30
tcctcaacaacggaccaccagagccgcacaaacggcttttttgtgcccg

SEQ ID NO: 610 - DNA, 49 bp, Artificial Sequence
  T2 hinge molecule for logic gate 2_31
tataaacagttcgttaagcgtcatacataaataaacatcgaggcttatc

SEQ ID NO: 611 - DNA, 40 bp, Artificial Sequence
  T2 hinge molecule for logic gate 2_32
ttaaacaaccgaagcatcttagtttccattaaacgggtgt

SEQ ID NO: 612 - DNA, 30 bp, Artificial Sequence
  T2 hinge molecule for logic gate 2_33
accagttgaggctttcaccctttctctgaa

SEQ ID NO: 613 - DNA, 44 bp, Artificial Sequence
  T2 hinge molecule for logic gate 2_34
cctcagaccggaattcctcaacggaaccagtaacattgatgata

SEQ ID NO: 614 - DNA, 44 bp, Artificial Sequence
  T2 hinge molecule for logic gate 2_35
taccgtattacccaaaggagccccgctaggttaatgctgacgag

SEQ ID NO: 615 - DNA, 43 bp, Artificial Sequence
  T2 hinge molecule for logic gate 2_36
ttttttttcagggatagcaagcccttgtcgaggtgccgttttt

SEQ ID NO: 616 - DNA, 35 bp, Artificial Sequence
  Staple of logic gate 3_1
ctcccttcccgttttttttttcgagggtccagtta

SEQ ID NO: 617 - DNA, 53 bp, Artificial Sequence
  Staple of logic gate 3_2
attaaagaacgtggactattaataaccagccaaagggaataagggcgatcaag

SEQ ID NO: 618 - DNA, 34 bp, Artificial Sequence
  Staple of logic gate 3_3
ctgagaatagtgtatcttcggggatccagttcga

SEQ ID NO: 619 - DNA, 19 bp, Artificial Sequence
  Staple of logic gate 3_4
tttttatccggtaactacg

SEQ ID NO: 620 - DNA, 58 bp, Artificial Sequence
  Staple of logic gate 3_5
tttttgtcgtttccttaaagggtgcagttacccttacctggagtagtcagctattttt

SEQ ID NO: 621 - DNA, 37 bp, Artificial Sequence
  Staple of logic gate 3_6
gagcaaaaggccgtccgaagtcaatagcggtacctgt

SEQ ID NO: 622 - DNA, 31 bp, Artificial Sequence
  Staple of logic gate 3_7
gctacgctagctcctggggagtttctcacgg

SEQ ID NO: 623 - DNA, 39 bp, Artificial Sequence
  Staple of logic gate 3_8
tttttgcatcgtggtgtcacgctcgtcgtttctttttca

SEQ ID NO: 624 - DNA, 56 bp, Artificial Sequence
  Staple of logic gate 3_9
tgtccttacatacagcgcgttttttttttaaacccgacagccttcgtacttttttt

SEQ ID NO: 625 - DNA, 60 bp, Artificial Sequence
  Staple of logic gate 3_10
gagtcatgcgaacatggtatctatgggggcatagataagatactaacttgtaagattgtt

SEQ ID NO: 626 - DNA, 52 bp, Artificial Sequence
  Staple of logic gate 3_11
cattgtaaggtatgcctaactgcatgcgagccaacgaccgggcaatattttt

SEQ ID NO: 627 - DNA, 53 bp, Artificial Sequence
  Staple of logic gate 3_12
ccgaaatcgtgacccaccagttaagcggatacatatttgtcatggtttcattc

SEQ ID NO: 628 - DNA, 45 bp, Artificial Sequence
  Staple of logic gate 3_13
cacggcgcgggggattggttgattacgtgatgaagtccttcttca

SEQ ID NO: 629 - DNA, 48 bp, Artificial Sequence
  Staple of logic gate 3_14
aaatcaaaatctttttttttttggccccagtgtgaagcatcagttttt

SEQ ID NO: 630 - DNA, 44 bp, Artificial Sequence
  Staple of logic gate 3_15
aacgacacgtggcgagaaaggaatgactccattaaaacaatagg

SEQ ID NO: 631 - DNA, 40 bp, Artificial Sequence
  Staple of logic gate 3_16
accacttaaaggtaccagacagtggcgataaccgtgatcg

SEQ ID NO: 632 - DNA, 40 bp, Artificial Sequence
  Staple of logic gate 3_17
ttaccggaacgcataatggctaagggaaccgtaaaaaggc

SEQ ID NO: 633 - DNA, 50 bp, Artificial Sequence
  Staple of logic gate 3_18
actttaaaccaactgatcttcagcatcttttggtcctctaataccttttt

SEQ ID NO: 634 - DNA, 42 bp, Artificial Sequence
  Staple of logic gate 3_19
ttttttaagcgttaatattttgcgcagaagtggtttgttttt

SEQ ID NO: 635 - DNA, 46 bp, Artificial Sequence
  Staple of logic gate 3_20
gcaggactcagatacgtggtcaccagtgccagagctaacgtgaggc

SEQ ID NO: 636 - DNA, 40 bp, Artificial Sequence
  Staple of logic gate 3_21
ggggcagacgcatctccaggattagcagagcggcatgagg

SEQ ID NO: 637 - DNA, 53 bp, Artificial Sequence
  Staple of logic gate 3_22
taagttgcgtgcacagtgctcatcattggaaaacgttgcggcgacgttatcac

SEQ ID NO: 638 - DNA, 40 bp, Artificial Sequence
  Staple of logic gate 3_23
cagctcatagtccactttcatccatagttgccgggaagaa

SEQ ID NO: 639 - DNA, 39 bp, Artificial Sequence
  Staple of logic gate 3_24
ttggtagcgtgcttggcgcggaaagactataaagatacc

SEQ ID NO: 640 - DNA, 40 bp, Artificial Sequence
  Staple of logic gate 3_25
ctagacgagctacagatgattttttttttgtcaggttttg

SEQ ID NO: 641 - DNA, 70 bp, Artificial Sequence
  Staple of logic gate 3_26
tttttaccatactaactttagcaaaaggccatttcccgcagactaatggcgcttccgcctttcgttttaa

SEQ ID NO: 642 - DNA, 39 bp, Artificial Sequence
  Staple of logic gate 3_27
ggtctgtaattttttttttttatcgaggaattatatcgc

SEQ ID NO: 643 - DNA, 42 bp, Artificial Sequence
  Staple of logic gate 3_28
tttttaaacctgcaactttatccgaaaagtgccacctttttt

SEQ ID NO: 644 - DNA, 42 bp, Artificial Sequence
  Staple of logic gate 3_29
ttttttgttttacaaggagtcttaaatgcccacaacattttt

SEQ ID NO: 645 - DNA, 42 bp, Artificial Sequence
  Staple of logic gate 3_30
tgtaacccactgccgcagtcgagttgctcttgcccggcgtca

SEQ ID NO: 646 - DNA, 68 bp, Artificial Sequence
  Staple of logic gate 3_31
catgatcccccatgtgcaaaatcatttccccgcctccatccagtctccaacgttcgcgttgagtaaac

SEQ ID NO: 647 - DNA, 45 bp, Artificial Sequence
  Staple of logic gate 3_32
cgcctatctcagcatgacctatataacacacaggttgtgcttttt

SEQ ID NO: 648 - DNA, 42 bp, Artificial Sequence
  Staple of logic gate 3_33
tttttgagcaaaaacaggaagtgtgcaaaaaagcggtttttt

SEQ ID NO: 649 - DNA, 60 bp, Artificial Sequence
  Staple of logic gate 3_34
gataccgcggcaaaatagggttgatagataactacgataggaaagcgcccccgcactcag

SEQ ID NO: 650 - DNA, 72 bp, Artificial Sequence
  Staple of logic gate 3_35
agctccggttcccaacgacacggatagaaaaagggaagctagagtaagggaacaagtttttaacatgaagtt

SEQ ID NO: 651 - DNA, 40 bp, Artificial Sequence
  Staple of logic gate 3_36
cccccctggtatcctactactaaaactgcgacaggcgttt

SEQ ID NO: 652 - DNA, 64 bp, Artificial Sequence
  Staple of logic gate 3_37
accgtactcccgcgcgcctaggcctggcgtatgaaagtagcacacgggcagtaatatgggaggg

SEQ ID NO: 653 - DNA, 55 bp, Artificial Sequence
  Staple of logic gate 3_38
tttttgtacgggcaccctaggtgcagattttttttttattcagctcttctagctt

SEQ ID NO: 654 - DNA, 48 bp, Artificial Sequence
  Staple of logic gate 3_39
tgttgccattgttttttttttctattatcaggttactgtcatgttttt

SEQ ID NO: 655 - DNA, 42 bp, Artificial Sequence
  Staple of logic gate 3_40
tttttaaatcgacctggtttaaattggagcgactgctttttt

SEQ ID NO: 656 - DNA, 59 bp, Artificial Sequence
  Staple of logic gate 3_41
tagtaatttatggttcattcgttttttttttttaagtagacgccatttcacaatttttt

SEQ ID NO: 657 - DNA, 61 bp, Artificial Sequence
  Staple of logic gate 3_42
atgaaaatcaagtaacgcgaccctactgtcgattacaacctactcggcaacggcaagggac

SEQ ID NO: 658 - DNA, 38 bp, Artificial Sequence
  Staple of logic gate 3_43
taggcggagccgctgttaacctggggatgccatcatct

SEQ ID NO: 659 - DNA, 40 bp, Artificial Sequence
  Staple of logic gate 3_44
gaaaggtaacccggtaccttcggagtttttttgtttgcaa

SEQ ID NO: 660 - DNA, 54 bp, Artificial Sequence
  Staple of logic gate 3_45
ttttttatgccgcttttgaggtcgaacccccaattccttattggttcgttacac

SEQ ID NO: 661 - DNA, 42 bp, Artificial Sequence
  Staple of logic gate 3_46
ttttttttctagaggtgtaaggatcagtgggaagatgttttt

SEQ ID NO: 662 - DNA, 52 bp, Artificial Sequence
  Staple of logic gate 3_47
atgctgatgcccgcagtaaagactgtattctgttagttacaactggtttttt

SEQ ID NO: 663 - DNA, 52 bp, Artificial Sequence
  Staple of logic gate 3_48
tttttgcgccacatagcagaatacgggacgatcgttgtcagaaggcgagtta

SEQ ID NO: 664 - DNA, 58 bp, Artificial Sequence
  Staple of logic gate 3_49
tttttaaccaccaacgtcgctgatggcccactacgtggacagttaccaatggcttttt

SEQ ID NO: 665 - DNA, 39 bp, Artificial Sequence
  Staple of logic gate 3_50
cccgtgacgctcacgctgtaggtaagtcgtatggggaat

SEQ ID NO: 666 - DNA, 48 bp, Artificial Sequence
  Staple of logic gate 3_51
tttttttccatatccactaatcgacgattttttttttggcaagtactc

SEQ ID NO: 667 - DNA, 38 bp, Artificial Sequence
  Staple of logic gate 3_52
tctcatagatatcctctatgttagtggtggtaggcggt

SEQ ID NO: 668 - DNA, 65 bp, Artificial Sequence
  Staple of logic gate 3_53
tactcttcggtatggctatggcaggctgttgagcgaaaactctcaaggtctgtgactggtttttt

SEQ ID NO: 669 - DNA, 36 bp, Artificial Sequence
  Staple of logic gate 3_54
ccggtcaagacgcaattatgggggtcatgtctccca

SEQ ID NO: 670 - DNA, 47 bp, Artificial Sequence
  Staple of logic gate 3_55
tttttccatccgtaagatgctttatcttacccactgcataagtcatt

SEQ ID NO: 671 - DNA, 44 bp, Artificial Sequence
  Staple of logic gate 3_56
ccgtcgtggtgttgttccagttttagttcgcgctcacatactca

SEQ ID NO: 672 - DNA, 51 bp, Artificial Sequence
  Staple of logic gate 3_57
gcagcagtcctttgcttatcgccactggcatccaccctcatgtggtcgttc

SEQ ID NO: 673 - DNA, 48 bp, Artificial Sequence
  Staple of logic gate 3_58
tttttatgcttgacggcgggagggcttttttttttttaccagaataga

SEQ ID NO: 674 - DNA, 39 bp, Artificial Sequence
  Staple of logic gate 3_59
gttccacctaacaaaatagcctgctgcgatcagttccat

SEQ ID NO: 675 - DNA, 55 bp, Artificial Sequence
  Staple of logic gate 3_60
cagcattggagtcaaattcgagggataaagacggcagctttcacccttctttttt

SEQ ID NO: 676 - DNA, 38 bp, Artificial Sequence
  Staple of logic gate 3_61
attgaacgagcgggcgctaggggatctgtcgtatatat

SEQ ID NO: 677 - DNA, 32 bp, Artificial Sequence
  Staple of logic gate 3_62
ataggggttccgcgcagccgcaaaagccggaa

SEQ ID NO: 678 - DNA, 36 bp, Artificial Sequence
  Staple of logic gate 3_63
aaatttttgttaaatagcaatatgttgcctaaacaa

SEQ ID NO: 679 - DNA, 44 bp, Artificial Sequence
  Staple of logic gate 3_64
gggccgagttaaaatcaaaggggcacctatctcagccgctggca

SEQ ID NO: 680 - DNA, 42 bp, Artificial Sequence
  Staple of logic gate 3_65
tttttcaggcttaatcagtgagcgaaaaaccgtctatttttt

SEQ ID NO: 681 - DNA, 55 bp, Artificial Sequence
  Staple of logic gate 3_66
acctagattaaagggacggcgaactgctttgcggggcaacatttttggtcttttt

SEQ ID NO: 682 - DNA, 45 bp, Artificial Sequence
  Staple of logic gate 3_67
cgcgttgcgtcgtgaggcttgaacgcctgctctcctgtcggtgta

SEQ ID NO: 683 - DNA, 45 bp, Artificial Sequence
  Staple of logic gate 3_68
tttttgagtactcaaccaattctcgttattgatattatctgcaat

SEQ ID NO: 684 - DNA, 46 bp, Artificial Sequence
  Staple of logic gate 3_69
aagactctaatgtgtgagaggacactgaagaagtcgggaggaaaac

SEQ ID NO: 685 - DNA, 71 bp, Artificial Sequence
  Staple of logic gate 3_70
ttttttattttgtagttgccgttgagagagagttcgcgacgtaggtcctaggtttttttcaagatcgttga

SEQ ID NO: 686 - DNA, 76 bp, Artificial Sequence
  Staple of logic gate 3_71
tttttcaaacaaaccaccgctggttcagtggaatcgtcttgagtccaacacatgctgctccgaaccccccgttcag

SEQ ID NO: 687 - DNA, 42 bp, Artificial Sequence
  Staple of logic gate 3_72
tttttcacgcgttaccgcatacgacaagtcagaggggttttt

SEQ ID NO: 688 - DNA, 45 bp, Artificial Sequence
  Staple of logic gate 3_73
taacgctgcatcgccacaaaaatcgacgctcaagtcagagttttt

SEQ ID NO: 689 - DNA, 44 bp, Artificial Sequence
  Staple of logic gate 3_74
tctcatgatagtttgcgcaacgtccgagatcccttatgatcttc

SEQ ID NO: 690 - DNA, 43 bp, Artificial Sequence
  Staple of logic gate 3_75
ttaagggatagatttagagagattttttttttttatcaaaaag

SEQ ID NO: 691 - DNA, 42 bp, Artificial Sequence
  Staple of logic gate 3_76
tttttctgggttagctccttcactttcaccagcgtttttttt

SEQ ID NO: 692 - DNA, 60 bp, Artificial Sequence
  Staple of logic gate 3_77
tttcttgcttctcagcacgtttgagttaatcggtgtatggaacatggaccatctacggcc

SEQ ID NO: 693 - DNA, 32 bp, Artificial Sequence
  Staple of logic gate 3_78
actgagcatgagtccgagcgccacatcgtaat

SEQ ID NO: 694 - DNA, 29 bp, Artificial Sequence
  Staple of logic gate 3_79
gcataggtcctaaaaaaccacgcctccac

SEQ ID NO: 695 - DNA, 36 bp, Artificial Sequence
  Staple of logic gate 3_80
gatggagggtgatctgctcacctcagtaaatttctg

SEQ ID NO: 696 - DNA, 45 bp, Artificial Sequence
  Staple of logic gate 3_81
cgcccacgccccctaaattcatgcggcgttagatagaatgtgcgg

SEQ ID NO: 697 - DNA, 45 bp, Artificial Sequence
  Staple of logic gate 3_82
aggaaacggctacactagaagagcgctatagtatgagcctgccgc

SEQ ID NO: 698 - DNA, 37 bp, Artificial Sequence
  Staple of logic gate 3_83
tacttctcagggagactaaatcggaacccccttttaa

SEQ ID NO: 699 - DNA, 40 bp, Artificial Sequence
  Staple of logic gate 3_84
ttaaatcacgaggtgcagcgaaagaccgccactgcatata

SEQ ID NO: 700 - DNA, 36 bp, Artificial Sequence
  Staple of logic gate 3_85
ttggtctaaccatcagtgtaggcgcttaatgcgggc

SEQ ID NO: 701 - DNA, 21 bp, Artificial Sequence
  Staple of logic gate 3_86
acgcgctgcgctctgctgaag

SEQ ID NO: 702 - DNA, 43 bp, Artificial Sequence
  Staple of logic gate 3_87
agcataaatgcgattggtgagctgggctccccctgcttcggct

SEQ ID NO: 703 - DNA, 21 bp, Artificial Sequence
  Staple of logic gate 3_88
gcgttaacgataggcaaacca

SEQ ID NO: 704 - DNA, 45 bp, Artificial Sequence
  Staple of logic gate 3_89
tgatcctgagtcaccccaagttttttggggtatctaaatatttcg

SEQ ID NO: 705 - DNA, 24 bp, Artificial Sequence
  Staple of logic gate 3_90
gtccaactgaccttctgaagctcc

SEQ ID NO: 706 - DNA, 39 bp, Artificial Sequence
  Staple of logic gate 3_91
ctcgtgcggctatcggtacgctgttttccataggctccg

SEQ ID NO: 707 - DNA, 39 bp, Artificial Sequence
  Staple of logic gate 3_92
agagcgagagcagccatttggtatcagaaaaatcggatt

SEQ ID NO: 708 - DNA, 41 bp, Artificial Sequence
  T0-hinge molecule for logic gate 3_1
accctaatcgtttcctctgcaaagtcattgctatcaagagg

SEQ ID NO: 709 - DNA, 52 bp, Artificial Sequence
  T0-hinge molecule for logic gate 3_2
tcgacgctcactaggagtgcccacaagtgtttctgtggcggagagctaaggt

SEQ ID NO: 710 - DNA, 61 bp, Artificial Sequence
  T0-hinge molecule for logic gate 3_3
tgttatctcgagaggtgaaccgagcgggagatgaatccgcctatcctgccctcacattttt

SEQ ID NO: 711 - DNA, 45 bp, Artificial Sequence
  T0-hinge molecule for logic gate 3_4
ctatcttgtctaagtccacccgcccggtcacgctgcgcgtttttt

SEQ ID NO: 712 - DNA, 26 bp, Artificial Sequence
  T0-hinge molecule for logic gate 3_5
cgtgaaggtaactggcctctggtctg

SEQ ID NO: 713 - DNA, 46 bp, Artificial Sequence
  T0-hinge molecule for logic gate 3_6
cggctcttcaaggtatgctactgactgtttttaggcctggagttaa

SEQ ID NO: 714 - DNA, 55 bp, Artificial Sequence
  T0-hinge molecule for logic gate 3_7
gtgtgcacacccatgcgttcggcaccagttaagacacgaatcttttcctccataa

SEQ ID NO: 715 - DNA, 37 bp, Artificial Sequence
  T0-hinge molecule for logic gate 3_8
cctccgcatacggggtctgacgcagcggtgaaaagag

SEQ ID NO: 716 - DNA, 43 bp, Artificial Sequence
  T0-hinge molecule for logic gate 3_9
cgtaaagcagatatcaactgccataaggatctcaagaagaatt

SEQ ID NO: 717 - DNA, 25 bp, Artificial Sequence
  T0-hinge molecule for logic gate 3_10
ttctatacggatgcacattgagacg

SEQ ID NO: 718 - DNA, 43 bp, Artificial Sequence
  T0-hinge molecule for logic gate 3_11
acaattccaatggagaacagtactggtaaagtcatgaatctac

SEQ ID NO: 719 - DNA, 43 bp, Artificial Sequence
  T2-hinge molecule for logic gate 3_1
accctaatcgtttcctctgcaaattgtcattgctatcaagagg

SEQ ID NO: 720 - DNA, 54 bp, Artificial Sequence
  T2-hinge molecule for logic gate 3_2
tcgacgctcactaggagtgcccacaagtgtttctgtggcggagattgctaaggt

SEQ ID NO: 721 - DNA, 63 bp, Artificial Sequence
  T2-hinge molecule for logic gate 3_3
tgttatctcgagaggtgaaccgagttcgggagatgaatccgcctatcctgccctcacattttt

SEQ ID NO: 722 - DNA, 47 bp, Artificial Sequence
  T2-hinge molecule for logic gate 3_4
ctatcttgtttctaagtccacccgcccggtcacgctgcgcgtttttt

SEQ ID NO: 723 - DNA, 28 bp, Artificial Sequence
  T2-hinge molecule for logic gate 3_5
cgtgaaggtttaactggcctctggtctg

SEQ ID NO: 724 - DNA, 48 bp, Artificial Sequence
  T2-hinge molecule for logic gate 3_6
cggctcttttcaaggtatgctactgactgtttttaggcctggagttaa

SEQ ID NO: 725 - DNA, 57 bp, Artificial Sequence
  T2-hinge molecule for logic gate 3_7
gtgtgcacacccatgcgttcggcaccagttaagacacgaatcttttcttctccataa

SEQ ID NO: 726 - DNA, 39 bp, Artificial Sequence
  T2-hinge molecule for logic gate 3_8
cctccgcatttacggggtctgacgcagcggtgaaaagag

SEQ ID NO: 727 - DNA, 45 bp, Artificial Sequence
  T2-hinge molecule for logic gate 3_9
cgtaaagcagatatcaactgccatttaaggatctcaagaagaatt

SEQ ID NO: 728 - DNA, 27 bp, Artificial Sequence
  T2-hinge molecule for logic gate 3_10
ttctatattcggatgcacattgagacg

SEQ ID NO: 729 - DNA, 45 bp, Artificial Sequence
  T2-hinge molecule for logic gate 3_11
acaattccaatggagaacagtactggtaaagtcatgttaatctac

SEQ ID NO: 730 - DNA, 26 bp, Artificial Sequence
  AntiCD3 adapter sequence
gcgagagtggagatggtgagtaaggc

SEQ ID NO: 731 - DNA, 47 bp, Artificial Sequence
  Strand with antiCD3 adapter sequence
tctcagtttccgacctccgaagcgagagtggagatggtgagtaaggc

SEQ ID NO: 732 - DNA, 26 bp, Artificial Sequence
  AntiCD19 adapter sequence
gcagtaaagttcaatagggtcaaagc

SEQ ID NO: 733 - DNA, 56 bp, Artificial Sequence
  Module for switchable antiCD19 antibody configuration with antiCD19 adapter sequence_1
gcagtaaagttcaatagggtcaaagcgctccaaacgggtcggttttctactctgga

SEQ ID NO: 734 - DNA, 56 bp, Artificial Sequence
  Module for switchable antiCD19 antibody configuration with antiCD19 adapter sequence_2
gcagtaaagttcaatagggtcaaagctgtattaatgttgacggctccagatttatc

SEQ ID NO: 735 - DNA, 58 bp, Artificial Sequence
  Module for switchable antiCD19 antibody configuration with antiCD19 adapter sequence_3
gcagtaaagttcaatagggtcaaagcacgcgcgcatagcctgattgaaaccgcgccgg

SEQ ID NO: 736 - DNA, 27 bp, Artificial Sequence
  Module for switchable antiCD19 antibody configuration with antiCD19 adapter sequence_4
tttttcgcgtccccggtcttgatccgg

SEQ ID NO: 737 - DNA, 27 bp, Artificial Sequence
  Module for switchable antiCD19 antibody configuration with antiCD19 adapter sequence_5
tttttactgggaagcggccagtattta

SEQ ID NO: 738 - DNA, 27 bp, Artificial Sequence
  Module for switchable antiCD19 antibody configuration with antiCD19 adapter sequence_6
gatgttcttgaatttgtcgccgttttt

SEQ ID NO: 739 - DNA, 27 bp, Artificial Sequence
  Module for switchable antiCD19 antibody configuration with antiCD19 adapter sequence_7
gtgttccgagtcccgaccgctgttttt

SEQ ID NO: 740 - DNA, 56 bp, Artificial Sequence; identical to SEQ ID NO: 733
  Module for non-switchable antiCD19 antibody configuration with antiCD19 adapter sequence_1

SEQ ID NO: 741 - DNA, 53 bp, Artificial Sequence
  Module for non-switchable antiCD19 antibody configuration with antiCD19 adapter sequence_2
gcagtaaagttcaatagggtcaaagcgatgttcttgaatttgtcgccgttttt

SEQ ID NO: 742 - DNA, 53 bp, Artificial Sequence
  Module for non-switchable antiCD19 antibody configuration with antiCD19 adapter sequence_3
gcagtaaagttcaatagggtcaaagcgtgttccgagtcccgaccgctgttttt

SEQ ID NO: 743 - DNA, 27 bp, Artificial Sequence
  Module for non-switchable antiCD19 antibody configuration with antiCD19 adapter sequence_4
tttttcgcgtccccggtcttgatccgg

SEQ ID NO: 744 - DNA, 27 bp, Artificial Sequence
  Module for non-switchable antiCD19 antibody configuration with antiCD19 adapter sequence_5
tttttactgggaagcggccagtattta

(19) United States
(12) Patent Application Publication
(10) Pub. No.: US 2022/0339181 A1
(43) Pub. Date: Oct. 27, 2022

(54) CONDITIONALLY SWITCHABLE NANOSTRUCTURE

(71) Applicants: TILIBIT NANOSYSTEMS GMBH, MÜNCHEN (DE); TECHNISCHE UNIVERSITÄT MÜNCHEN, MÜNCHEN (DE)

(72) Inventors: JONAS JOERG FUNKE, GARCHING (NEAR MUNICH) (DE); KLAUS WAGENBAUER, GARCHING (NEAR MUNICH) (DE); HENDRIK DIETZ, HAAR (DE); BENJAMIN KICK, MOOSBURG (DE); JEAN-PHILIPPE SOBCZAK, MÜNCHEN (DE); PHILIP KETTERER, MÜNCHEN (DE)

(21) Appl. No.: 17/620,851

(22) PCT Filed: Jun. 22, 2020
(86) PCT No.: PCT/EP2020/067329

§ 371 Date: Dec. 20, 2021

(30) Foreign Application Priority Data
Jun. 21, 2019 (EP) 19181646.1

(51) Int. Cl. A61K 31/711; C12Q 1/6804

(52) U.S. Cl. CPC A61K 31/711; C12Q 1/6804; B82Y 5/00

(57) ABSTRACT

The present invention relates to a conditionally switchable nanostructure configured to assume a first configuration and a second configuration, wherein the nanostructure comprises a binding site that is configured to bind to a binding target, and wherein the accessibility of the binding site for the binding target in the second configuration is different to the accessibility of the binding site for the binding target in the first configuration. Further, the nanostructure is configured to assume the first configuration when none of the coupling sites of the coupling site set is coupled to its respective coupling target and to assume the second configuration when each coupling site of a subset of the coupling site set is coupled to its respective coupling target, wherein the subset comprises at least one coupling site. Additionally, the present technology also relates to a system comprising the nanostructure as well as a method utilizing the nanostructure or the system. Finally, the nanostructure may be comprised in a substance or composition for use as a medicament.

The present invention generally relates to nanostructures. In particular embodiments, the present invention relates to the field of nanorobotics. Nanorobotics relates to nanoscale devices that can be programmed and/or designed to perform specific tasks.

The field of nanostructures is becoming more and more relevant in the field of technological applications. The miniaturization of devices is an increasing trend for example in the field of information technology, sensing or medical applications.

Typically, nanostructures are manufactured using technically advanced, highly complex manufacturing processes, that may limit the yield and efficiency and generally is associated with high efforts. However, in recent years new methods for manufacturing nanostructures have been developed. These include methods for manufacturing nucleic acid nanostructures as disclosed in U.S. Pat. No. 7,842,793 B3. The disclosure relates to methods and compositions for generating nanoscale devices, systems, and enzyme factories based upon a nucleic acid nanostructure that can be designed to have a predetermined structure. This technique is widely known as “DNA origami”. While the present invention will mainly be described with regard to DNA origami structures, it should be understood that this is merely exemplary and that the present invention can also be practiced with other nanostructures.

DNA origami is a technology that allows the production of 3D structures on the nanometer scale from DNA molecules. Chemical modifications or single-stranded DNA overhangs can be placed on origami structures with positional control. DNA origami structures can be easily linked to multiple molecules, each of which mediates a specific molecular interaction, thus endowing the DNA origami structure with the interaction capabilities of the linked molecules. In biological systems, molecular complexes with multiple interactions play a role in enabling complex functionalities.

Recently, these techniques have been used for the realization of different nanoscale devices. For example, U.S. Pat. No. 9,863,930 B2 discloses various molecular barcoded bi-stable switches that can be used to detect various analytes, wherein the molecular barcoded bi-stable switch may be manufactured using DNA origami.

Further, WO 2012/061719 A2 discloses DNA origami devices useful in the targeted delivery of biologically active entities to specific cell populations. While this device may enable a targeted delivery of biologically active entities, it relies on specific interaction molecules, i.e. aptamers, that may bind to two different binding targets. This may be disadvantageous since it can restrict the possible applications of such a device.

In light of the above, it is an object of the present invention to overcome or at least alleviate the shortcomings and disadvantages of the prior art. That is, it is an object of the present invention to provide a conditionally switchable nanostructure.

These objects are met by the present invention.

In some embodiment the present invention provides a nanostructure comprising a binding site configured to bind to a binding target, wherein the nanostructure is configured to assume a first configuration and a second configuration, wherein the accessibility of the binding site for the binding target in the second configuration is different to the accessibility of the binding site for the binding target in the first configuration. The nanostructure further comprises a coupling site set comprising at least one coupling site, wherein each coupling site is configured to couple to a respective coupling target. Further, the nanostructure is configured to assume the second configuration when each coupling site of a subset of the coupling site set is coupled to its respective coupling target, wherein the subset comprises at least one coupling site. The nanostructure is further configured to assume the first configuration when none of the coupling sites of the coupling site set is coupled to its respective coupling target.

Generally, it should be understood that the nanostructure comprising a binding site should not be construed to mean that the nanostructure only comprises this binding site. Instead, the nanostructure may also have more than one binding site. In other words (unless explicitly states or unless clear to the skilled person), usage of an article (e.g., “a”, “an”) should not be understood to exclude the plural. For example, also a nanostructure comprising two binding sites is to be construed to be encompassed by “a nanostructure comprising a binding site”.

In other words, the nanostructure may be configured to assume different configurations including a first configuration A and a second configuration B. In some embodiments, these configurations may also be realized as an “open” and “closed” configuration. Depending on the configuration the nanostructure assumes, the binding site may be change its accessibility. For example, the nanostructure may be more accessible in the second configuration than in the first configuration.

Further, the nanostructure may also comprise at least one coupling site (i.e., one coupling site or a plurality of coupling sites). Depending on whether or not these coupling sites are coupled to coupling targets, a likelihood of the nanostructure assuming the different configurations may change. As an example, when all (or a part) of the coupling sites are coupled to their coupling targets, the nanostructure may be more likely to assume the second configuration than would be the case when none of the coupling sites are coupled to their coupling targets.

Thus, depending on the coupling state of the coupling sites, the accessibility of the binding site (or the binding sites) may be changed. Thus, the coupling state of the coupling sites may “activate” and/or “deactivate” the accessibility of the binding site. It will be understood that this may also include that the nanostructure is “further activated”, i.e., that the activity of the nanostructure is further increased.

Thus, depending on the coupling state of the coupling sites, in simplified words, the nanostructure may be switched between different states. This allows many different applications. In particular, such a nanostructure can be used to couple to particular coupling targets and to only be “active” once coupled to such coupling targets. This may, e.g., be used to couple the nanostructures to particular cells and to activate it once coupled to such cells.

It should be understood that the subset of the coupling site set may also coincide with the coupling site set.

Such a nanostructure may thus allow to at least significantly change the probability of being in the first or second configuration depending on the coupling of the coupling sites. Further, a great variety of interactions may be utilized in such a device which may be advantageous compared to the state of the art that relies on specific and limited interactions.

The coupling sites may not be configured to couple to each other. Thus, “self coupling” of the nanostructure to itself may be prohibited.

In some embodiments, each coupling site may be configured to only couple to a single type of coupling target. That is, a coupling site may not couple to distinct coupling targets.

The nanostructure may comprise a plurality of binding sites, wherein each of the binding site may be configured to bind to a respective binding target, and wherein the accessibility of each of the binding sites for the respective binding target in the second configuration may be different to the accessibility in the first configuration.

A different accessibility between the two configurations may generally be advantageous since it allows to condition the accessibility of the binding site on the configuration assumed by the nanostructure, which in turn may be conditioned on the presence or absence of coupling targets. Thus, such a nanostructure may for example enable targeted drug delivery or other forms of treatments of a disease.

The nanostructure may be configured to assume the first configuration with a higher probability than the second configuration when none of the coupling sites of the coupling site set are coupled to its respective coupling target.

That is, also when none of the coupling sites of the coupling site set are coupled to their respective coupling targets, the nanostructure may assume both the first configuration and the second configuration. More particularly, there may be an equilibrium between the first configuration and the second configuration in this binding state. However, in this equilibrium, the first configuration may be “preferred”, i.e., the nanostructure may assume this first configuration with a higher likelihood than the second configuration in this coupling state.

Further, the binding site may be accessible for the binding target in one configuration and may not be accessible for the binding target in the other configuration. That is, the binding site may only be accessible in one of the two configurations. This may be particularly beneficial for applications where the accessibility should be conditioned on the configuration assumed by the nanostructure as it may essentially inhibit accessibility in one configuration.

The nanostructure may be configured to assume first and second equilibrium states between the first configuration and the second configuration, wherein the probability that the nanostructure assumes the second configuration may be different (e.g., higher) in the second equilibrium state than in the first equilibrium state. The nanostructure may be configured to assume the first equilibrium state when all of the respective coupling targets are absent, i.e. none of the coupling sites of the coupling site set can couple to the respective coupling target and to assume the second equilibrium state when all of the respective coupling targets of the subset of coupling sites are present, i.e. all coupling sites of the subset of coupling sites can couple to the respective coupling targets.

That is, the nanostructure may generally be in an equilibrium state between the first configuration and the second configuration, wherein it may assume each configuration with a certain probability. However, in some embodiments an equilibrium state may comprise a probability of essentially zero for being in one of the configurations. In other words, in such an equilibrium state the nanostructure may essentially always remain in one configuration.

The probability of the nanostructure assuming the first configuration in the first equilibrium state may be at least 0.3, preferably at least 0.5, more preferably at least 0.7, such as at least 0.9 and the probability of the nanostructure assuming the second configuration in the first equilibrium state may be at most 0.7, preferably at most 0.5, more preferably at most 0.3, such as at most 0.1.

Further, the probability of the nanostructure assuming the first configuration in the second equilibrium state may be at most 0.7, preferably at most 0.5, more preferably at most 0.3, such as at most 0.1 and the probability of the nanostructure assuming the second configuration in the second equilibrium state may be at least 0.3, preferably at least 0.5, more preferably at least 0.7, such as at least 0.9.

In some embodiments, the nanostructure may comprise a first portion and a second portion, wherein the first portion may be movable with respect to the second portion. Moreover, the first portion and the second portion may each comprise at least one of the coupling sites of the coupling site set and at least one of the first portion and the second portion may comprise the binding site.

Further, the subset of the coupling site set may comprise at least one coupling site comprised by the first portion and at least one coupling site comprised by the second portion. That is, each portion may comprise at least one coupling site.

The first portion and the second portion may be movably attached to each other, e.g. by means of a hinge or a rotational axis.

In some embodiments, the nanostructure may comprise at least one additional portion.

The nanostructure may comprise a maximum length, i.e. a length corresponding to the largest extent of the nanostructure, and the maximum length may be smaller than 1000 nm, preferably smaller than 500 nm, such as 100 nm.

The binding site may bind reversibly to the binding target, i.e. they can bind and detach repeatedly.

Similarly, at least one of the coupling sites of the coupling site set may couple reversibly to the respective coupling target, i.e. they can couple and decouple repeatedly.

In some embodiments, the coupling site set may comprise only identical coupling sites, i.e. coupling sites configured to couple to the same coupling targets. In other embodiments, the coupling site set may comprise at least two distinct coupling sites, configured to couple to distinct coupling targets.

Further, the nanostructure may be configured to couple to coupling targets comprised by a single entity. In other embodiments, the nanostructure may be configured to couple to coupling targets comprised by a plurality of entities. In such cases, the plurality of entities may comprise at least two distinct entities.

The binding target may be comprised by an entity also comprising at least one coupling target.

In some embodiments, the nanostructure may be at least partially formed by a DNA origami structure.

That is, readily available DNA origami techniques that involve comparatively less complex procedures to assemble than standard nanomanufacturing techniques may be used to manufacture the nanostructure.

The DNA origami structure may comprise at least one scaffolding strand, i.e. single-stranded polynucleotide scaffold DNA with a known sequence.

The DNA origami structure may further comprise a plurality of single-stranded oligonucleotide staple strands, wherein each staple strand may be at least partially complementary to at least one scaffolding strand.

Further, each of the staple strands may be configured to bind to the at least one scaffolding strand in two distinct places, wherein the at least one scaffolding strand may be folded and/or arranged such that the desired nanostructure may be formed.

In some embodiments, at least one binding site may be a molecule.

Further, at least one binding site may be configured to bind to a CD28 protein as a binding target. The at least one binding site may also be configured to bind another specific cluster of differentiation (CD) molecule and/or to another disease-associated cell surface molecule.

Similarly, at least one coupling site comprised by the coupling site set may be a molecule.

In some embodiments, at least one coupling site of the coupling site set may be configured to couple to a CD3 antigen as a coupling target.

Further, at least one coupling site of the coupling site set may be configured to couple to an epithelial cell adhesion molecule as a coupling target.

In embodiments, where at least one binding site and/or at least one coupling site is a molecule, said molecule may be bound to the nanostructure by means of linker molecules.

That is, a great number and variety of interactions may be realised for binding sites and/or coupling sites. This may provide an advantage compared to the known state of the art, e.g.

WO 2012/061719 A2, as it may overcome the disadvantage of limited interactions and thus provide a nanostructure for various different applications.

Moreover, in embodiments where the nanostructure is at least partially formed by a DNA origami structure and wherein the DNA origami structure comprises at least one scaffolding strand and a plurality of staple strands, the molecule may be bound to one of the at least one scaffolding strand or a staple strand by means of a linker molecule, wherein the linker molecule may be connected to a DNA strand portion, which is complementary to a portion of the at least one scaffolding strand or to a portion of a staple strand.

In embodiments where the nanostructure comprises a first and second portion, the first portion and the second portion may comprise an identical shape. Further, the shape may be a cuboid, i.e. a rectangular box.

In addition, the two portions may each comprise a cavity, configured to form a chamber when the nanostructure is in the first configuration. Further, at least one binding site may be located in the cavity of the first portion.

Yet further, at least one binding site may be attached to an outer surface of the cavity by means of a rod, wherein the binding site may be configured to leave the cavity when the nanostructure is in the second configuration.

The person skilled in the art will appreciate that a rod may be any type of flexible link between a portion of the cavity and at least one binding site.

In some embodiments in which the nanostructure comprises a first and a second portion, the first portion may be a rod. The rod may comprise a first recess at a first longitudinal end of the rod and a second recess at a second longitudinal end of the rod, each recess comprising a respective bottom surface. The recesses may be oriented perpendicular to a longitudinal axis of the rod and preferably facing in opposite directions, and wherein the bottom surfaces may lie in parallel planes.

In some embodiments, one recess may comprise at least one of the coupling sites and the other recess may comprise at least one binding site.

In some embodiments in which the nanostructure comprises a first and a second portion, the second portion may be a hollow disc. The hollow disc may comprise two outer discs and at least one connection structure connecting the two outer discs to form the hollow disc.

Further, each outer disc may comprise a disc recess. The outer discs may be connected to form the hollow disc such that the two disc recesses may be located at an angle offset of 180° with respect to each other.

In some embodiments wherein the nanostructure comprises a rod, the rod may be located within the disc and the rod may rotate freely around a rotation axis through the centre of the hollow disc, and the disc recesses and the recesses of the rod may be configured to provide access to the coupling site and the at least one binding site in one rotational position of the rod, which may be the second configuration.

The outer disc may comprise a coupling site at an outer rim and adjacent to the disc recess that may be configured to provide access to the coupling site comprised by the recess of the rod.

In some embodiments, the binding site may be more accessible for the binding target in the second configuration than in the first configuration.

However, in other embodiments, the binding site may be more accessible for the binding in the first configuration than in the second configuration.

The coupling site set may be formed by one coupling site. However, in other embodiments, the coupling site set may also be formed by a plurality of coupling sites.

In a further embodiment the present invention relates to a system comprising the nanostructure as described above.

The system may comprise at least one binding target.

The system may compromise a plurality of coupling targets.

The system may comprise at least one first entity. Further, each of the at least one first entity may comprise at least one coupling target. Yet further, each of the at least one first entity may comprise at least one binding target.

In some embodiments, the system may comprise at least one second entity. Further, each of the at least one second entity may comprise at least one coupling target.

The at least one coupling target comprised by the second entity may be distinct to the at least one coupling target comprised by the first entity.

Each of the at least one first entity may be a cell. Further, each of the at least one first entity may be a T-cell.

Similarly, each of the at least one second entity may be a cell. Further, each of the at least one second entity may be a tumor cell.

In some embodiments, the at least one coupling target comprised by each of the at least one first entity may be a CD3 antigen.

Further, the at least one coupling target comprised by each of the at least one second entity may be an epithelial cell adhesion molecule.

Yet further, the at least one binding target may be a CD28 protein.

In a further embodiment, the present invention relates to a method for conditionally binding a binding site to a binding target, wherein the method comprises utilizing a nanostructure or a system according to the present invention.

The method may comprise the nanostructure assuming the first configuration and the second configuration, wherein the accessibility of the binding site for the binding target in the second configuration may be different to the accessibility of the binding site for the binding target in the first configuration, wherein the nanostructure may assume the first configuration when none of the coupling sites of the coupling site set is coupled to its respective coupling target and wherein the nanostructure may assume the second configuration when each of the coupling sites of the subset of the coupling site set is coupled to its respective coupling target.

The method may comprise assuming the first configuration with a higher probability than the second configuration when none of the coupling sites of the coupling site set is coupled to its respective coupling target.

Further, the method may comprise the nanostructure assuming a first and second equilibrium state between a first configuration of the nanostructure and a second configuration of the nanostructure, wherein the probability of assuming the second configuration may be different (e.g., higher) in the second equilibrium state than in the first equilibrium state. Further the method may comprise the nanostructure assuming the first equilibrium state when all of the respective coupling targets are absent, i.e. none of the coupling sites of the coupling site set can couple to the respective coupling target and the nanostructure assuming the second equilibrium state when all of the respective coupling targets of the subset of coupling sites are present, i.e. all coupling sites of the subset of coupling sites can couple to the respective coupling targets.

The method may comprise coupling the coupling sites to identical coupling targets. Alternatively, the method may comprise coupling the coupling sites to distinct coupling targets.

In some embodiments, the method may not comprise coupling the coupling sites to each other. That is, the coupling sites may not be configured to couple to each other or in other words, a coupling site may not also be configured as coupling target.

The method may comprise coupling to coupling targets comprised by a single entity.

Additionally or alternatively, the method may comprise coupling to coupling targets comprised by distinct entities.

In embodiments, wherein the method comprises utilizing a system wherein each of the at least one first entity is a T-cell, the binding target binding to the binding site may at least further activate the T-cell.

In a further embodiment, the present invention relates to a substance comprising a plurality of nanostructures according to the present invention.

The substance may be for use as a medicament. This may be advantageous as the nanostructures may enabled targeted treatment of diseases, such as targeted drug delivery of cell activation.

Further, the substance may be for use in the treatment of cancer.

Additionally or alternatively, the substance may be for use in the treatment of blood clotting disorders.

Further, the substance may be for use in the treatment of immunological disorders.

Additionally or alternatively, the substance may be for use in the treatment of human immunodeficiency virus (HIV) infection.

Further, the substance may be for use in the treatment of macular degeneration.

Additionally or alternatively, the substance may be for use in the treatment of diabetes.

In a further embodiment, the present invention relates to a composition comprising a plurality of nanostructures according to the present invention.

The composition may be for use as a medicament. This may be advantageous as the nanostructures may enabled targeted treatment of diseases, such as targeted drug delivery of cell activation.

Further, the composition may be for use in the treatment of cancer.

Additionally or alternatively, the composition may be for use in the treatment of blood clotting disorders.

Further, the composition may be for use in the treatment of immunological disorders.

Additionally or alternatively, the composition may be for use in the treatment of human immunodeficiency virus (HIV) infection.

Further, the composition may be for use in the treatment of macular degeneration.

Additionally or alternatively, the composition may be for use in the treatment of diabetes.

Thus, in some embodiments the present invention provides a nanostructure that is configured to assume a first and a second configuration, wherein the nanostructure further comprises a binding site for which the accessibility is different between the first and second configuration. Furthermore, the nanostructure comprises a coupling site set comprising at least one coupling site, wherein the nanostructure is configured to assume the first configuration when none of the coupling sites is coupled to its respective coupling target and to assume the second configuration when each coupling site of a subset of the coupling site set is coupled to its respective coupling target.

In other words, the present invention discloses a conditionally switchable nanostructure that may assume different configurations depending on the coupling of at least a subset of the coupling site set comprised by the nanostructure, wherein the accessibility of the binding site may change depending on the configuration the nanostructure assumes. For example, the binding site may be significantly more accessible in one configuration than the other, in some cases it may even be only accessible in one of the two configurations. This may allow to enable access to the binding site only conditioned on the coupling of at least a subset of coupling sites of the coupling site set. This may be advantageous as it may control the access to the binding site based on the presence or absence of at least a subset of the respective coupling targets.

Furthermore, the nanostructure may generally utilize a great variety of interactions, for example the binding and/or coupling sites may be molecules, for example an antibody, an antibody fragment, a DNA strand, a biotin molecule, a streptavidin molecule, a maleimide-thiol chemistry molecule, a click chemistry molecule, etc. This may be advantageous compared to the state of the art such as the devices disclosed in WO 2012/061719 A2, that relies on specific and limited interactions.

Further, the nanostructure may at least partially be manufactured using DNA origami techniques. Owing to the self-assembly of DNA origami structures and the readily available software for designing the corresponding scaffolding and staple strands this may be a comparatively less complex manufacturing process compared to standard nanomanufacturing techniques.

Generally, it will be understood that embodiments of the present invention provide the ability to conditionally activate and deactivate certain molecular interactions based on the coupling state of other interaction sites.

According to some embodiments of the present invention, a DNA origami structure comprising rigid domains that are connected in such a way that the structure can adopt different conformations is provided. At least two interaction sites (e.g., interaction molecules) are attached to the structure, e.g., a coupling site and a binding site. The interaction molecules are attached in such a way, and the structure is designed in such a way, that when a subset A of the interaction molecules is not bound the intended targets, the structure spends the majority of time in a conformation in which a subset B of the interaction molecules cannot bind to their intended targets due to steric hindrance. Once the subset A of the interaction molecules binds to their intended targets, the accessible conformations of the structure are restricted due to the bound targets such that the structure now spends a larger amount of time than in the initial unbound state in conformations in which the subset B of interaction molecules can bind their intended targets. Therefore, the binding rate of the subset B of interaction molecules on the structure is conditionally modified by the binding state of the subset A of interaction molecules. Because the conditional activation is mediated through the shape of the phase space of the structure, and not through the interaction molecules themselves, the function can be applied to any type of interaction, such as antibodies, antibody fragments, DNA strands, biotin-streptavidin, maleimide-thiol chemistry, click chemistry, and many others.

Thus, in embodiments of the present invention, multiple molecular interaction sites are added to a DNA origami structure. Further, the molecular interactions are conditionally activated or deactivated based on the binding state of other molecular interactions present on the origami structure in a generalizable fashion.

In some embodiments of the present invention the nanostructure is comprised in a substance or composition that may be used as a medicament. This may be beneficial, as the nanostructure may enable targeted treatment of diseases. For example, through targeted drug delivery or targeted activation of T-cells. Such diseases may for example be different types of cancer, blood clotting disorders, immunological disorders, HIV infections, macular degeneration or diabetes. That is, embodiments of the present invention may enable treatment of a number of diseases.

Thus, owing to the wide range of interactions available, a more versatile and flexible conditionally switchable nanostructure is provided.

The present invention is also defined by the following numbered embodiments.

Below, reference will be made to nanostructure embodiments. These embodiments are abbreviated by the letter “N” followed by a number. Whenever reference is herein made to “nanostructure embodiments”, these embodiments are meant.

N1. A nanostructure (10) comprising
a binding site (12) configured to bind to a binding target (100);
wherein the nanostructure (10) is configured to assume a first configuration (A) and a second configuration (B), wherein the accessibility of the binding site (12) for the binding target (100) in the second configuration (B) is different to the accessibility of the binding site (12) for the binding target (100) in the first configuration (A);
wherein the nanostructure (10) comprises a coupling site set comprising at least one coupling site (14, 16), wherein each coupling site (14, 16) is configured to couple to a respective coupling target (202, 204);
wherein the nanostructure (10) is configured to assume the second configuration (B) when each coupling site (14, 16) of a subset of the coupling site set is coupled to its respective coupling target (202, 204), wherein the subset comprises at least one coupling site (14, 16); and
wherein the nanostructure (10) is configured to assume the first configuration (A) when none of the coupling sites (14, 16) of the coupling site set is coupled to its respective coupling target (202, 204).

It should be understood that the subset of the coupling site set may also coincide with the coupling site set.

N2. The nanostructure according to the preceding embodiment, wherein the coupling sites (14,16) are not configured to couple to each other.

N3. The nanostructure according to any of the preceding embodiments, wherein each coupling site (14, 16) is configured to only couple to a single type of coupling target (202, 204).

That is, a coupling site (14, 16) may not couple to distinct coupling targets (202, 204).

N4. The nanostructure (10) according to any of the preceding embodiments, wherein the nanostructure (12) comprises a plurality of binding sites (12),
wherein each of the binding sites (12) is configured to bind to a respective binding target (100), and
wherein the accessibility of each of the binding sites (12) for the respective binding target (100) in the second configuration (B) is different to the accessibility in the first configuration (A).

N5. The nanostructure according to any of the preceding embodiments, wherein the nanostructure (10) is configured to assume the first configuration (A) with a higher probability than the second configuration (B) when none of the coupling sites (14, 16) of the coupling site set is coupled to its respective coupling target (202, 204).

N6. The nanostructure according to any of the preceding embodiments, wherein the binding site (12) is accessible for the binding target (100) in one configuration (A, B), and is not accessible for the binding target (100) in the other configuration (B, A).

N7. The nanostructure according to any of the preceding embodiments, wherein the nanostructure (10) is configured to assume first and second equilibrium states between the first configuration (A) and the second configuration (B),
wherein the probability that the nanostructure (10) assumes the second configuration (B) is different (e.g., higher) in the second equilibrium state than in the first equilibrium state;
wherein the nanostructure (10) is configured to
   assume the first equilibrium state when all of the respective coupling targets (202, 204) are absent, i.e. none of the coupling sites (14, 16) of the coupling site set can couple to the respective coupling target (202, 204); and to
   assume the second equilibrium state when all of the respective coupling targets (202, 204) of the subset of coupling sites are present, i.e. all coupling sites (14,16) of the subset of coupling sites can couple to the respective coupling targets (202, 204).

N8. The nanostructure according to the preceding embodiment, wherein the probability of the nanostructure (10) assuming the first configuration (A) in the first equilibrium state is at least 0.3, preferably at least 0.5, more preferably at least 0.7, such as at least 0.9.

N9. The nanostructure according to any of the 2 preceding embodiments, wherein the probability of the nanostructure (10) assuming the second configuration (B) in the first equilibrium state is at most 0.7, preferably at most 0.5, more preferably at most 0.3, such as at most 0.1.

N10. The nanostructure according to any of the 3 preceding embodiments, wherein the probability of the nanostructure (10) assuming the first configuration (A) in the second equilibrium state is at most 0.7, preferably at most 0.5, more preferably at most 0.3, such as at most 0.1.

N11. The nanostructure according to any of the 4 preceding embodiments, wherein the probability of the nanostructure (10) assuming the second configuration (B) in the second equilibrium state is at least 0.3, preferably at least 0.5, more preferably at least 0.7, such as at least 0.9.

N12. The nanostructure according to any of the preceding embodiments, wherein the nanostructure (10) comprises a first portion (18) and a second portion (20);
wherein the first portion (18) is movable with respect to the second portion (20);
wherein the first portion (18) and the second portion (20) each comprise at least one of the coupling sites (14, 16) of the coupling site set; and
wherein at least one of the first portion (18) and the second portion (20) comprises the binding site (12).

N13. The nanostructure according to the preceding embodiment, wherein the subset of the coupling site set comprises at least one coupling site (14, 16) comprised by the first portion (18) and at least one coupling site (14, 16) comprised by the second portion (20).

N14. The nanostructure according to any of the 2 preceding embodiments, wherein the first portion (18) and the second portion (20) are movably attached to each other, e.g. by means of a hinge or a rotational axis.

N15. The nanostructure according to any of the 3 preceding embodiments, wherein the nanostructure (10) comprises at least one additional portion.

N16. The nanostructure according to any of the preceding embodiments, wherein the nanostructure comprises a maximum length, i.e. a length corresponding to the largest extend of the nanostructure, and wherein the maximum length is smaller than 1000 nm, preferably smaller than 500 nm, such as 100 nm.

N17. The nanostructure according to any of the preceding embodiments, wherein the binding site (12) can bind to the binding target (100) reversibly, i.e. they can bind and detach repeatedly.

N18. The nanostructure according to any of the preceding embodiments, wherein at least one of the coupling sites (14, 16) of the coupling site set couples reversibly to the respective coupling target (202, 204), i.e. they can couple and decouple repeatedly.

N19. The nanostructure according to any of the preceding embodiments, wherein the coupling site set comprises only identical coupling sites (14, 16), i.e. configured to couple to the same coupling targets (202, 204).

N20. The nanostructure according to any of the embodiments N1 to N18, wherein the coupling site set comprises at least two distinct coupling sites (14, 16), configured to couple to distinct coupling targets (202, 204).

N21. The nanostructure according to any of the preceding embodiments, wherein the nanostructure (10) is configured to couple to coupling targets (202, 204) comprised by a single entity (302).

N22. The nanostructure according to any of the preceding embodiments, wherein the nanostructure (10) is configured to couple to coupling targets (202, 204) comprised by a plurality of entities (302, 304).

N23. The nanostructure according to the preceding embodiment, wherein the plurality of entities (302, 304) comprises at least two distinct entities.

N24. The nanostructure according to any of the preceding embodiments, wherein the binding target (100) is comprised by an entity (302) also comprising at least one coupling target (202, 204).

N25. The nanostructure according to any of the preceding embodiments, wherein the nanostructure (10) is at least partially formed by a DNA origami structure.

N26. The nanostructure according to the preceding embodiment, wherein the DNA origami structure comprises at least one scaffolding strand, i.e. single-stranded polynucleotide scaffold DNA with a known sequence.

N27. The nanostructure according to the preceding embodiment, wherein the DNA origami structure further comprises a plurality of single-stranded oligonucleotide staple strands and wherein each staple strand is at least partially complementary to at least one scaffolding strand.

N28. The nanostructure according to the preceding embodiment, wherein each of the staple strands is configured to bind to the at least one scaffolding strand in two distinct places,
wherein the at least one scaffolding strand is folded and/or arranged such that the desired nanostructure (10) is formed.

N29. The nanostructure according to any of the preceding embodiments, wherein at least one binding site (12) is a molecule.

N30. The nanostructure according to any of the preceding embodiments, wherein at least one binding site (12) is configured to bind to a cluster differentiation (CD) molecule, e.g., a CD28 protein, and/or to another disease-associated cell surface molecule as a binding target (100).

N31. The nanostructure according to any of the preceding embodiments, wherein at least one coupling site (14, 16) comprised by the coupling site set is a molecule.

N32. The nanostructure according to any the preceding embodiments, wherein at least one coupling site (14, 16) of the coupling site set is configured to couple to a CD3 antigen as a coupling target (202, 204).

N33. The nanostructure according to any of the preceding embodiments, wherein at least one coupling site (14, 16) of the coupling site set is configured to couple to an epithelial cell adhesion molecule as a coupling target (202, 204).

N34. The nanostructure according to any of the preceding embodiments with the features of at least one of N29 and N31, wherein the molecule is bound to the nanostructure (10) by means of linker molecules.

N35. The nanostructure according to the preceding embodiment and with the features of embodiment N26 and N27, wherein the molecule is bound to one of the at least one scaffolding strand or a staple strand by means of a linker molecule;
wherein the linker molecule is connected to a DNA strand portion, which is complementary to a portion of the at least one scaffolding strand or to a portion of a staple strand.

N36. The nanostructure according to any of the preceding embodiments with the features of embodiment N12, wherein the first portion (18) and the second portion (20) comprise an identical shape.

N37. The nanostructure according to the preceding embodiment, wherein the shape is a cuboid, i.e. a rectangular box.

N38. The nanostructure according to any of the 2 preceding embodiments, wherein the two portions (18, 20) each comprise a cavity (36, 39), configured to form a chamber when the nanostructure (10) is in the first configuration (A).

N39. The nanostructure according to the preceding embodiment, wherein at least one binding site (12) is located in the cavity (36) of the first portion (18).

N40. The nanostructure according to the preceding embodiment, wherein at least one binding site (12) is attached to an outer surface of the cavity (36) by means of a rod (40),
wherein the binding site (12) is configured to leave the cavity (36) when the nanostructure is in the second configuration (B).

The person skilled in the art will appreciate that a rod (40) may be any type of flexible link between a portion of the cavity (36) and at least one binding site (12).

N41. The nanostructure according to any of the preceding embodiments with the features of embodiment N12, wherein the first portion (18) is a rod (18A).

N42. The nanostructure according to the preceding embodiment, wherein the rod (18A) comprises a first recess (181) at a first longitudinal end of the rod (18A) and a second recess (182) at a second longitudinal end of the rod (18A), each recess (181, 182) comprising a respective bottom surface (1811, 1821).

N43. The nanostructure according to the preceding embodiment, wherein the recesses (181, 182) are oriented perpendicular to a longitudinal axis of the rod and preferably facing in opposite directions, and wherein the bottom surfaces (1811, 1821) may lie in parallel planes.

N44. The nanostructure according to the preceding embodiment, wherein one recess (181) comprises at least one of the coupling sites (16) and the other recess (182) comprises at least one binding site (12).

N45. The nanostructure according to any of the preceding embodiments with the features of embodiment N12, wherein the second portion (20) is a hollow disc (20A).

N46. The nanostructure according to the preceding embodiment, wherein the hollow disc (20A) comprises two outer discs (21, 23) and at least one connection structure (25) connecting the two outer discs (21, 22) to form the hollow disc (20A).

N47. The nanostructure according to the preceding embodiment, wherein each outer disc (21, 23) comprises a disc recess (22, 24).

N48. The nanostructure according to the preceding embodiment, wherein the outer discs (21, 23) are connected to form the hollow disc (20A) such that the two disc recesses (22, 24) are located at an angle offset of 180° with respect to each other.

N49. The nanostructure according to the preceding embodiment with the features of embodiment N44, wherein the rod (18A) is located within the disc (20A) and

wherein the rod (18A) rotates freely around a rotation axis through the centre of the hollow disc (20); and

wherein the disc recesses (22, 24) and the recesses (181, 182) of the rod (18A) are configured to provide access to the coupling site (16) and the at least one binding site (12) in one rotational position of the rod (18A), which is the second configuration (B).

N50. The nanostructure according to the preceding embodiment, wherein the outer disc (23) comprises a coupling site (14) at an outer rim and adjacent to the disc recess (24) that is configured to provide access to the coupling site (16) comprised by the recess (181) of the rod (18A).

N51. The nanostructure (10) according to any of the preceding embodiments, wherein the binding site (12) is more accessible for the binding target (100) in the second configuration (B) than in the first configuration (A).

N52. The nanostructure (10) according to any of the embodiments N1 to N50, wherein the binding site (12) is more accessible for the binding (100) in the first configuration (A) than in the second configuration (B).

N53. The nanostructure (10) according to any of the preceding embodiments, wherein the coupling site set is formed by one coupling site.

N54. The nanostructure (10) according to any of the embodiments N1 to N52, wherein the coupling site set is formed by a plurality of coupling sites (14, 16).

Below, reference will be made to system embodiments. These embodiments are abbreviated by the letter “S” followed by a number. Whenever reference is herein made to “system embodiments”, these embodiments are meant.

S1. A system comprising the nanostructure according to any of the preceding nanostructure embodiments.

S2. The system according to the preceding system embodiment, wherein the system comprises at least one binding target (100).

S3. The system according to any of the preceding system embodiments, wherein the system comprises a plurality of coupling targets (202, 204).

S4. The system according to any of the preceding system embodiments, wherein the system comprises at least one first entity (302).

S5. The system according to the preceding system embodiment and with the features of embodiment S3, wherein each of the at least one first entity (302) comprises at least one coupling target (202, 204).

S6. The system according to any of the 2 preceding embodiments and with the features of S2, wherein each of the at least one first entity (302) comprises at least one binding target (100).

S7. The system according to any of the 3 preceding system embodiments, wherein the system comprises at least one second entity (304).

S8. The system according to the preceding system embodiment and with the features of embodiment S3, wherein each of the at least one second entity (304) comprises at least one coupling target (204).

S9. The system according to the preceding system embodiment and with the features of embodiment S5, wherein the at least one coupling target (204) comprised by the second entity (304) is distinct to the at least one coupling target (202) comprised by the first entity (302).

S10. The system according to any of the preceding system embodiments with the features of S4, wherein each of the at least one first entity (302) is a cell.

S11. The system according to the preceding system embodiment, wherein each of the at least one first entity (302) is a T-cell.

S12. The system according to any of the preceding system embodiments with the features of S7, wherein each of the at least one second entity (304) is a cell.

S13. The system according to the preceding system embodiment, wherein each of the at least one second entity (304) is a tumor cell.

S14. The system according to any of the preceding system embodiments with the features of embodiments S11 and S5, wherein the at least one coupling target (202, 204) comprised by each of the at least one first entity (302) is a CD3 antigen.

S15. The system according to any of the preceding system embodiments with the features of embodiment S12 and S8, wherein the at least one coupling target (202, 204) comprised by each of the at least one second entity (304) is an epithelial cell adhesion molecule.

S16. The system according to any of the preceding system embodiments with the features of embodiments S11 and S6, wherein the at least one binding target (100) is a CD28 protein.

Below, reference will be made to method embodiments. These embodiments are abbreviated by the letter “M” followed by a number. Whenever reference is herein made to “method embodiments”, these embodiments are meant.

M1. A method for conditionally binding a binding site (12) to a binding target (100), wherein the method comprises
utilizing a nanostructure (10) according to any of the preceding nanostructure embodiments or a system according to any of the preceding system embodiments.

M2. The method according to the preceding method embodiment, wherein the method comprises the nanostructure (10) assuming the first configuration (A) and the second configuration (B), wherein the accessibility of the binding site (12) for the binding target (100) in the second configuration (B) is different to the accessibility of the binding site (12) for the binding target (100) in the first configuration (A);
wherein the nanostructure (10) assumes the first configuration (A) when none of the coupling sites (14, 16) of the coupling site set is coupled to its respective coupling target (202, 204); and
wherein the nanostructure (10) assumes the second configuration (B) when each of the coupling sites (14, 16) of the subset of the coupling site set is coupled to its respective coupling target (202, 204).

M3. The method according to the preceding method embodiment, wherein the method comprises assuming the first configuration (A) with a higher probability than the second configuration (B) when none of the coupling sites (14, 16) of the coupling site set is coupled to its respective coupling target (202, 204).

M4. The method according to any of the preceding method embodiments, wherein the method comprises
the nanostructure (10) assuming a first and second equilibrium state between a first configuration (A) of the nanostructure (10) and a second configuration (B) of the nanostructure (10), wherein the probability of assuming the second configuration (B) is different (e.g., higher) in the second equilibrium state than in the first equilibrium state;
the nanostructure (10) assuming the first equilibrium state when all of the respective coupling targets (202, 204) are absent, i.e. none of the coupling sites (14, 16) of the coupling site set can couple to the respective coupling target (202, 204); and
the nanostructure (10) assuming the second equilibrium state when all of the respective coupling targets (202, 204) of the subset of coupling sites are present, i.e. all coupling sites (14,16) of the subset of coupling sites can couple to the respective coupling targets (202, 204).

M5. The method according to the preceding method embodiment, wherein the method comprises coupling the coupling sites (14, 16) to identical coupling targets (202, 204).

M6. The method according to any of the penultimate method embodiment, wherein the method comprises coupling the coupling sites (14, 16) to distinct coupling targets (202, 204).

M7. The method according to any of the preceding method embodiments, wherein the method does not comprise coupling the coupling sites (14, 16) to each other.

M8. The method according to any of the preceding method embodiments, wherein the method comprises coupling to coupling targets (202, 204) comprised by a single entity (302).

M9. The method according to any of the preceding method embodiments, wherein the method comprises coupling to coupling targets (202, 204) comprised by distinct entities (302, 304).

M10. The method according to the preceding embodiment, wherein the system comprises the features of embodiment S11,
wherein the binding target (100) binding to the binding site (12) at least further activates the T-cell.

Below, reference will be made to substance embodiments. These embodiments are abbreviated by the letter “T” followed by a number. Whenever reference is herein made to “substance embodiments”, these embodiments are meant.

T1. A substance comprising a plurality of nanostructures according to any of the preceding nanostructure embodiments.

T2. The substance according to the preceding substance embodiment for use as a medicament.

T3. The substance according to any of the preceding embodiments for use in the treatment of cancer.

T4. The substance according to any of the preceding substance embodiments for use in the treatment of blood clotting disorders.

T5. The substance according to any of the preceding substance embodiments for use in the treatment of immunological disorders.

T6. The substance according to any of the preceding substance embodiments for use in the treatment of human immunodeficiency virus (HIV) infection.

T7. The substance according to any of the preceding substance embodiments for use in the treatment of macular degeneration.

T8. The substance according to any of the preceding substance embodiments for use in the treatment of diabetes.

Below, reference will be made to composition embodiments. These embodiments are abbreviated by the letter “C” followed by a number. Whenever reference is herein made to “composition embodiments”, these embodiments are meant

C1. A composition comprising a plurality of nanostructures according to any of the preceding nanostructure embodiments.

C2. The composition according to the preceding composition embodiment for use as a medicament.

C3. The composition according to any of the preceding composition embodiments for use in the treatment of cancer.

C4. The composition according to any of the preceding composition embodiments for use in the treatment of blood clotting disorders.

C5. The composition according to any of the preceding composition embodiments for use in the treatment of immunological disorders.

C6. The composition according to any of the preceding composition embodiments for use in the treatment of human immunodeficiency virus (HIV) infection.

C7. The composition according to any of the preceding composition embodiments for use in the treatment of macular degeneration.

C8. The composition according to any of the preceding composition embodiments for use in the treatment of diabetes.

Embodiments of the present invention will now be described with reference to the accompanying drawings. These embodiments should only exemplify, but not limit, the present invention.

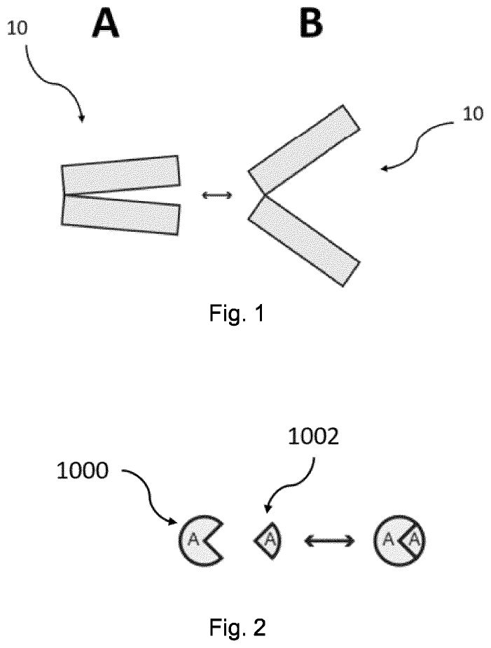

FIG. 1 depicts a nanostructure in two different configurations;

FIG. 2 depicts an interaction site and its interaction partner;

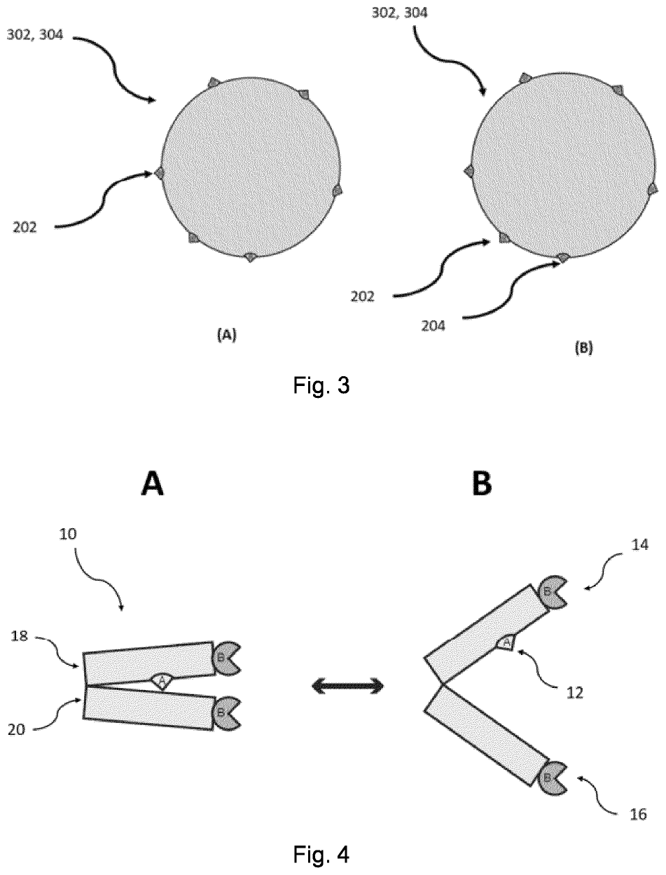

FIG. 3 depicts entities used in embodiments of the present technology;

FIG. 4 depicts a nanostructure according to a general embodiment;

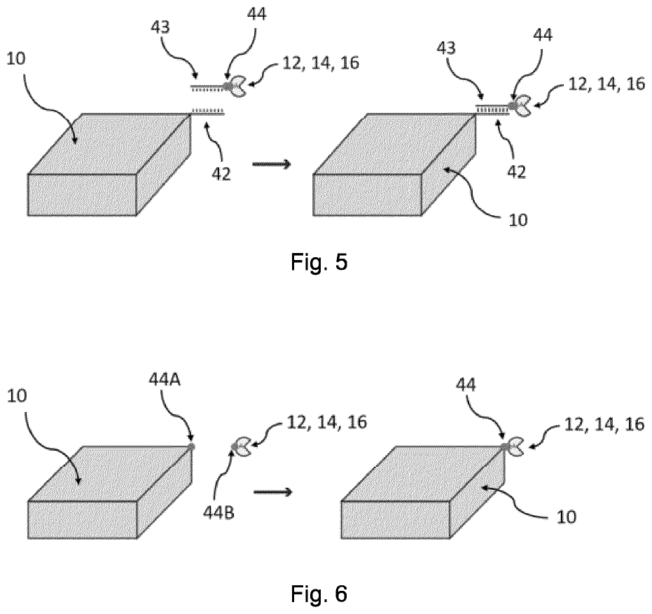

FIG. 5 depicts a mechanism for adding functional sites to a nanostructure;

FIG. 6 depicts another mechanism for adding functional sites to a nanostructure;

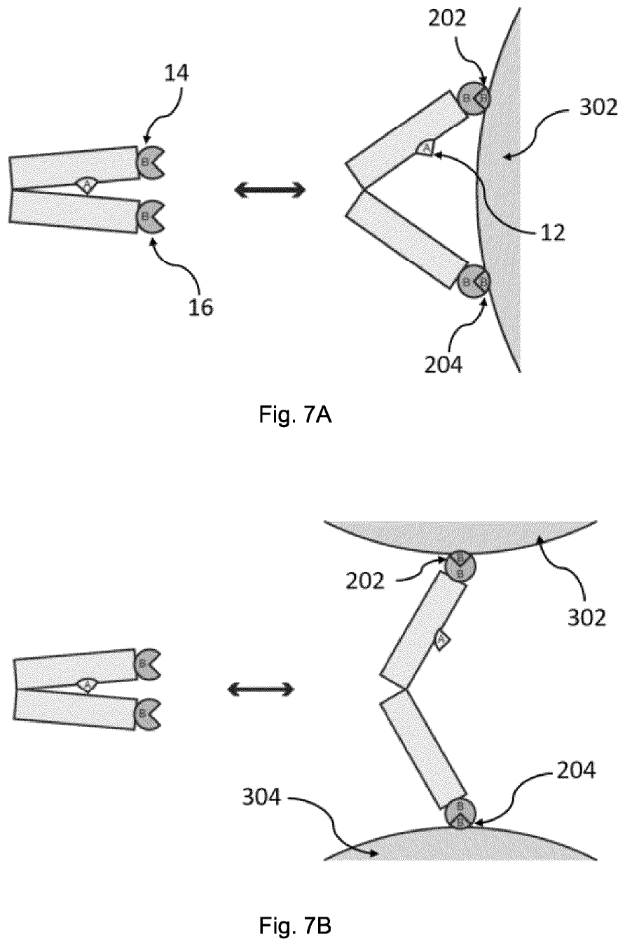

FIG. 7A depicts coupling of a nanostructure to a single entity;

FIG. 7B depicts coupling of a nanostructure to two entities;

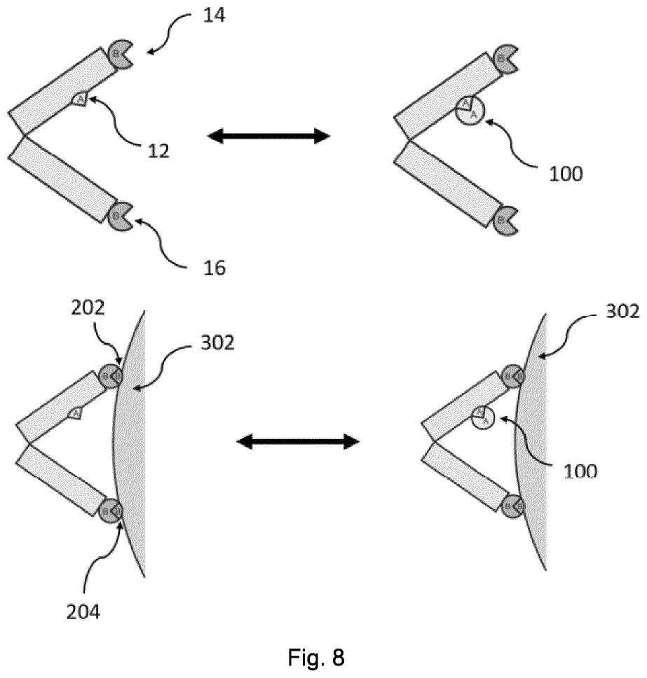

FIG. 8 depicts binding of a binding target to the nanostructure;

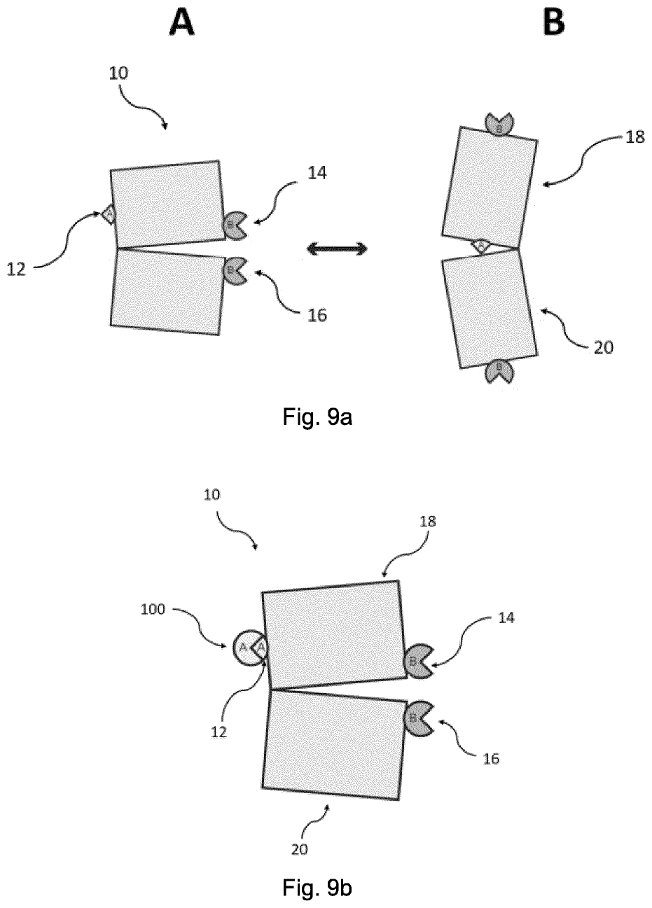

FIG. 9a, 9b depict an embodiment of a nanostructure wherein the binding site is more accessible in the first configuration;

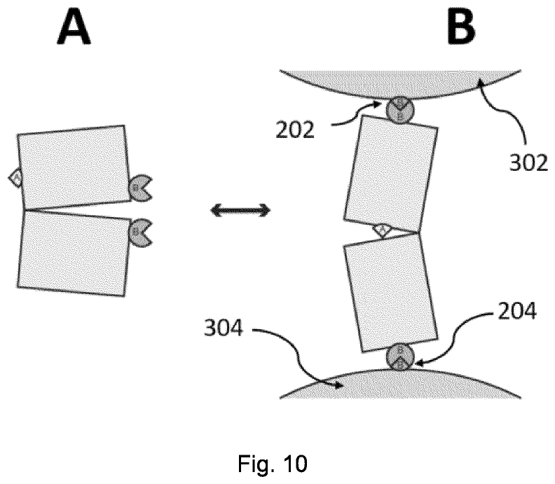

FIG. 10 depicts an equilibrium between different configuration of the nanostructure depicted in FIGS. 9a and 9b;

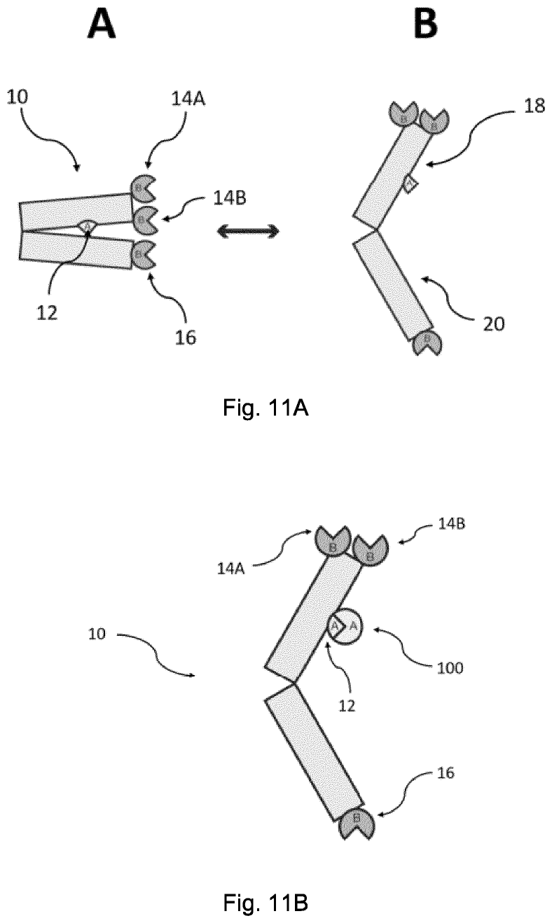

FIG. 11A depicts a nanostructure comprising a plurality of coupling sites in two configurations;

FIG. 11B depicts binding of a binding target to a nanostructure comprising a plurality of coupling sites;

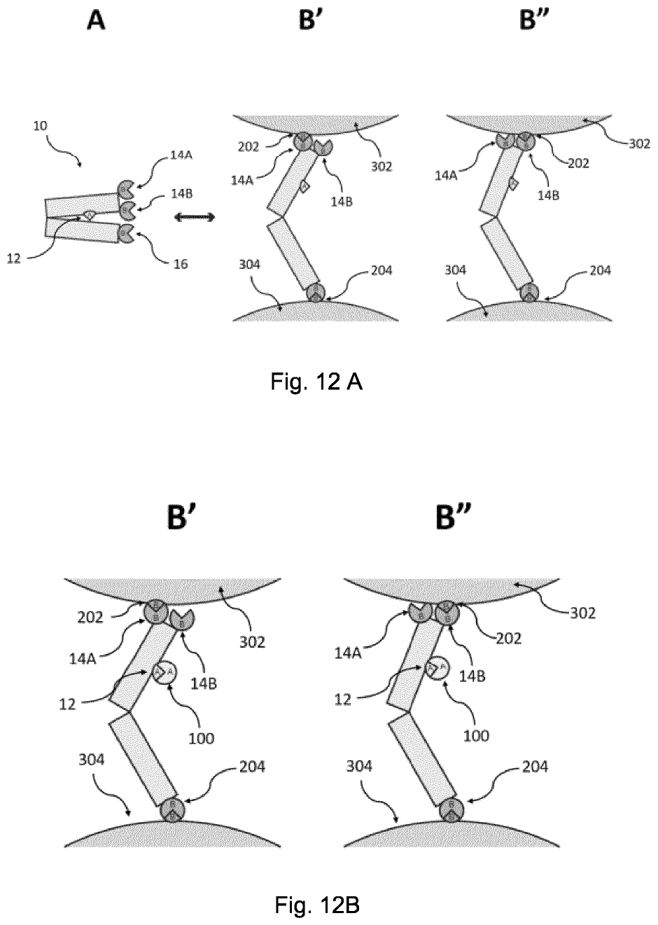

FIG. 12A depicts coupling of a nanostructure comprising a plurality of coupling sites to two entities.

FIG. 12B depicts binding of a binding target to a nanostructure coupled to two entities;

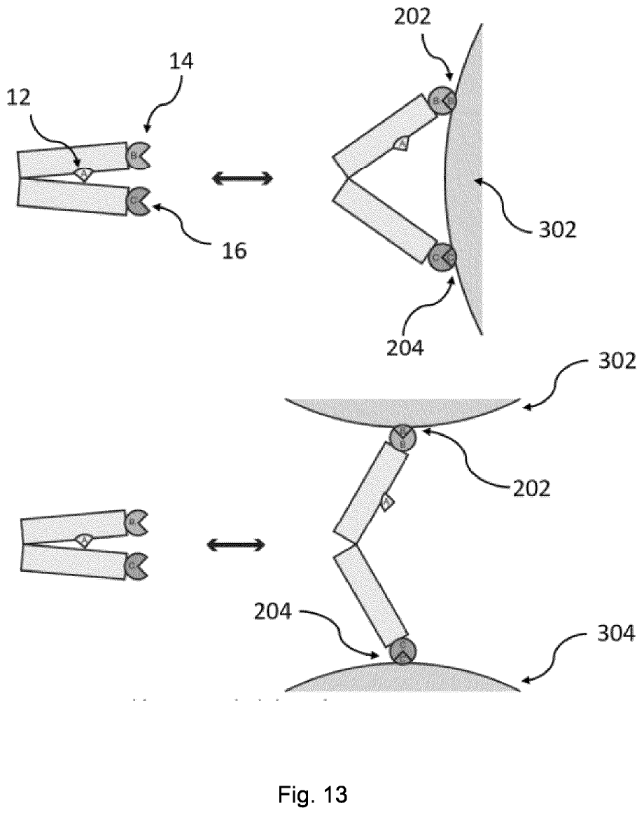

FIG. 13 depicts a nanostructure with distinct coupling sites;

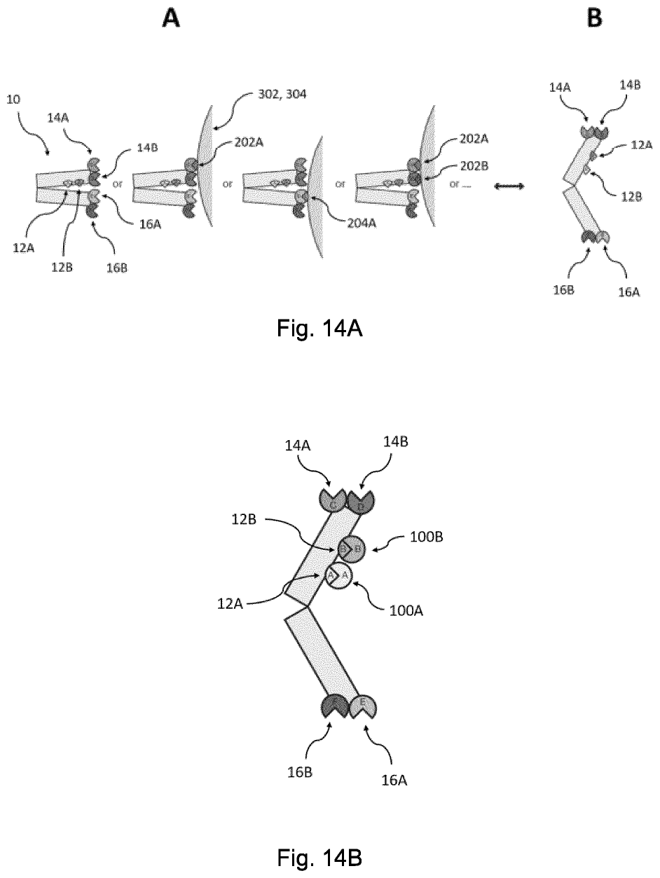

FIG. 14A depicts a nanostructure comprising a plurality of distinct coupling sites and distinct binding sites in two configurations;

FIG. 14B depicts binding of two distinct binding targets to a nanostructure;

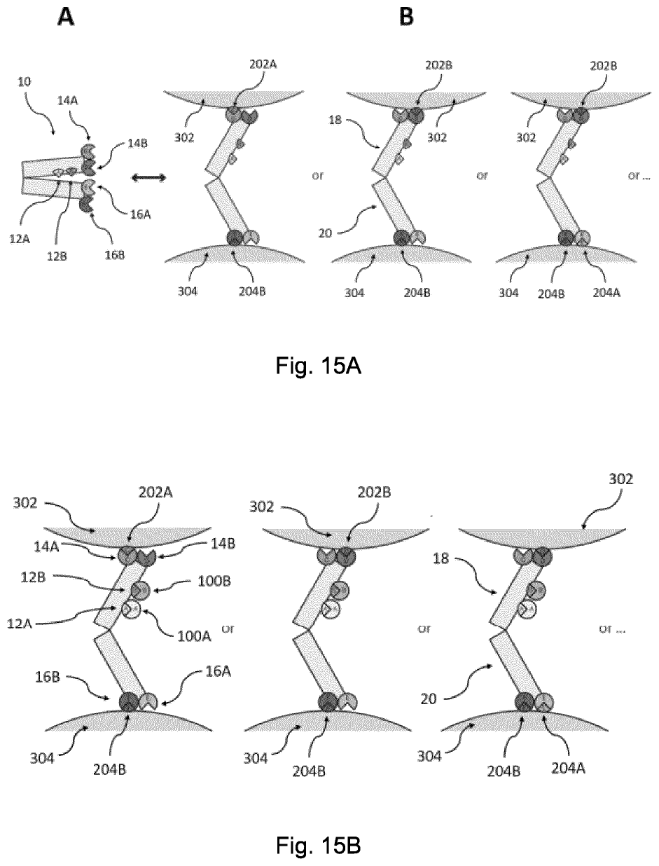

FIG. 15A depicts coupling of a nanostructure comprising a plurality of distinct coupling sites to two entities;

FIG. 15B depicts binding and coupling of a nanostructure comprising a plurality of distinct coupling sites and distinct binding sites in two configurations;

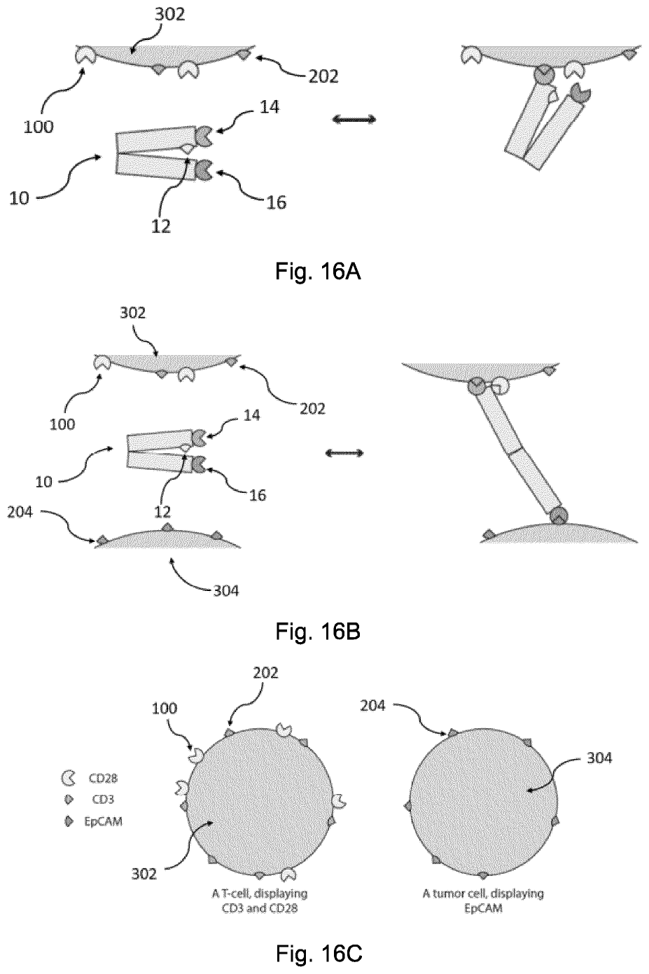

FIG. 16A depicts coupling of a single coupling site to a single entity;

FIG. 16B depicts coupling and binding of a nanostructure to two distinct entities;

FIG. 16C depicts two distinct entities;

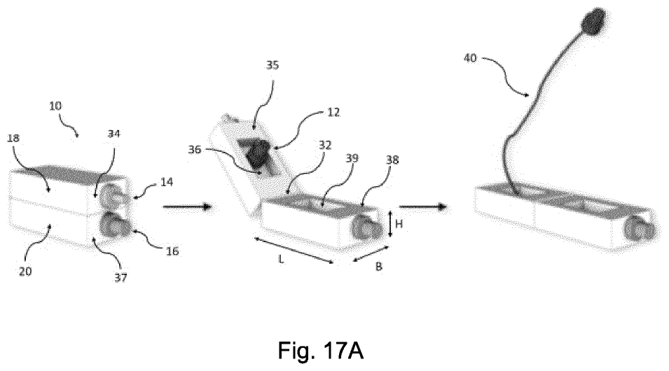

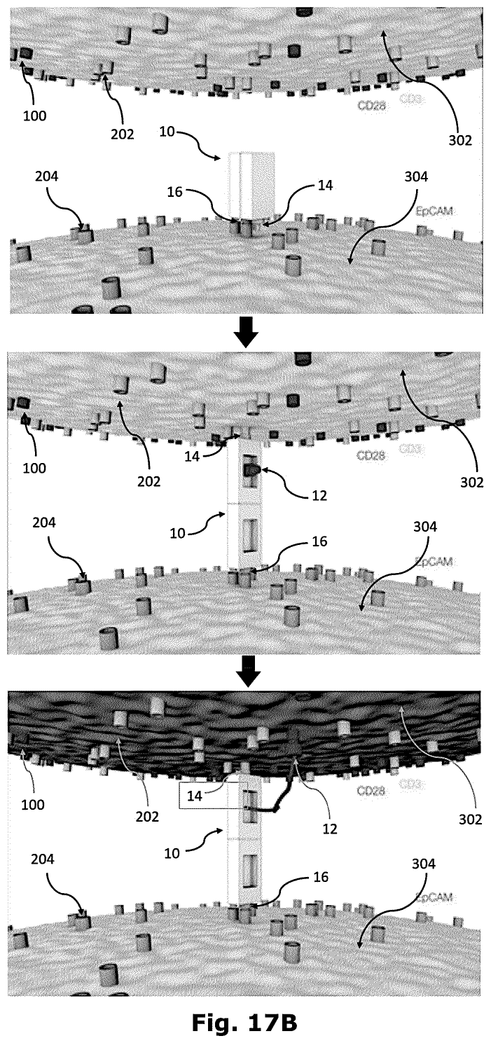

FIG. 17A depicts an implementation of a nanostructure as shown in FIGS. 10A and 10B;

FIG. 17B depicts the process of a nanostructure coupling and binding to two distinct entities;

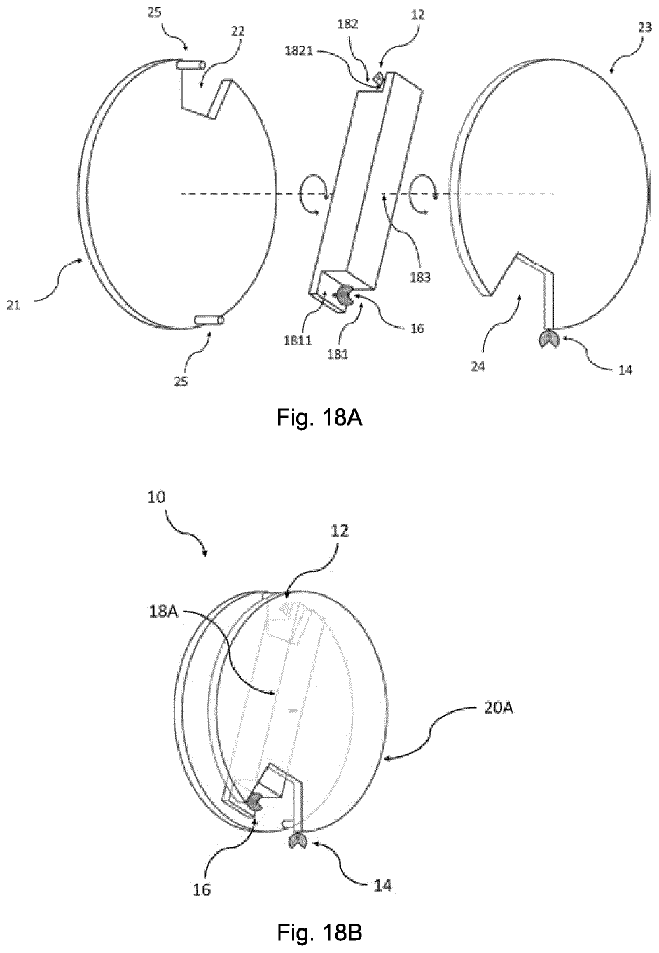

FIG. 18A depicts the portions of an implementation of a nanostructure;

FIG. 18B depicts an assembled implementation of a nanostructure;

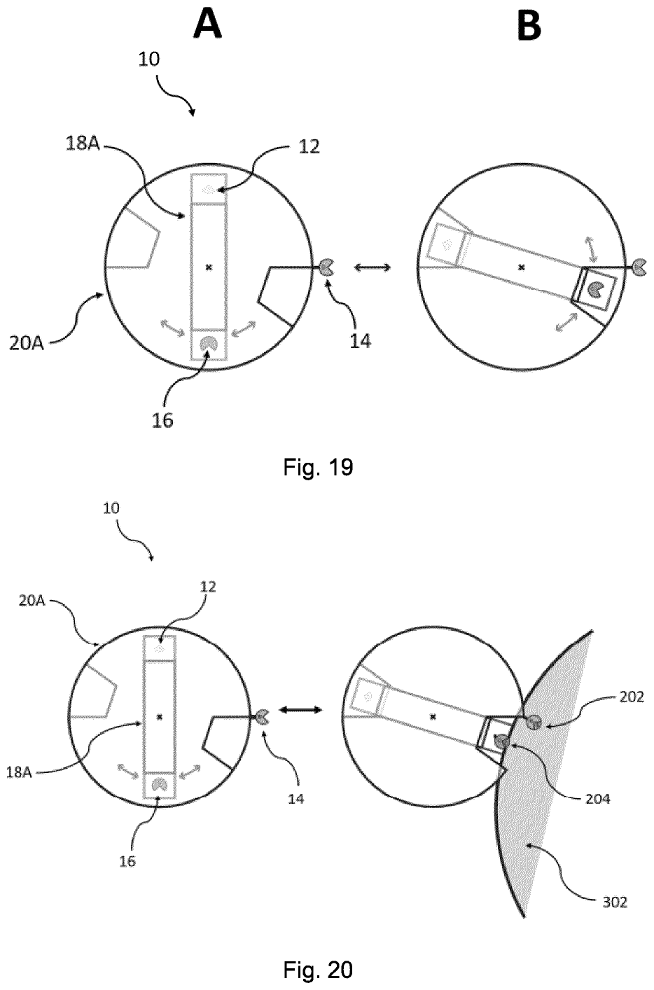

FIG. 19 depicts the embodiment of a nanostructure in two configurations;

FIG. 20 depicts the working principle of an implementation of a nanostructure;

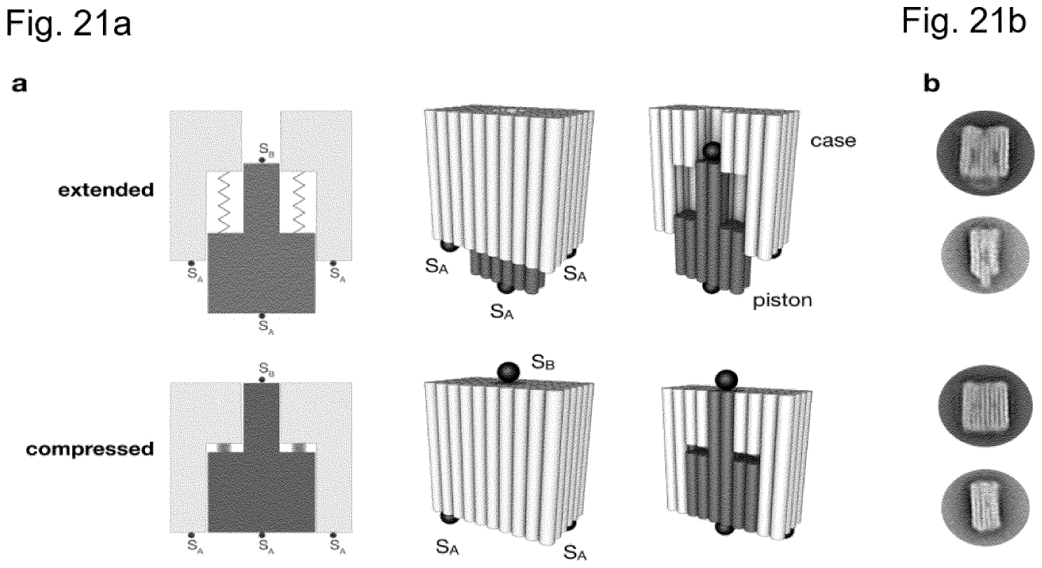

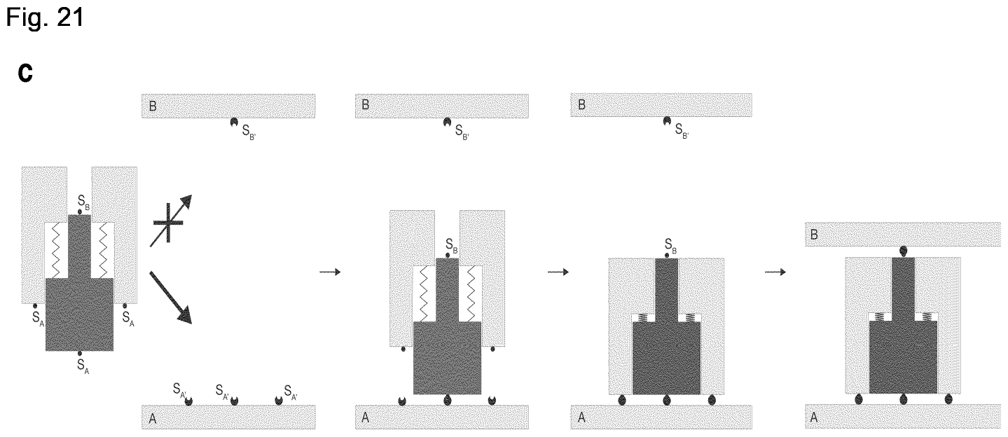

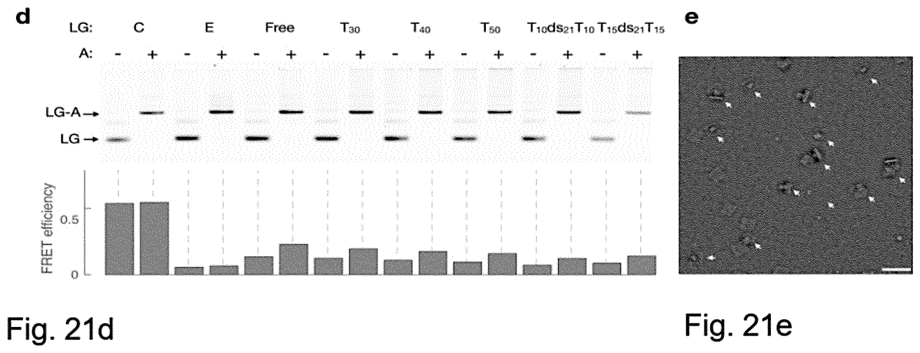

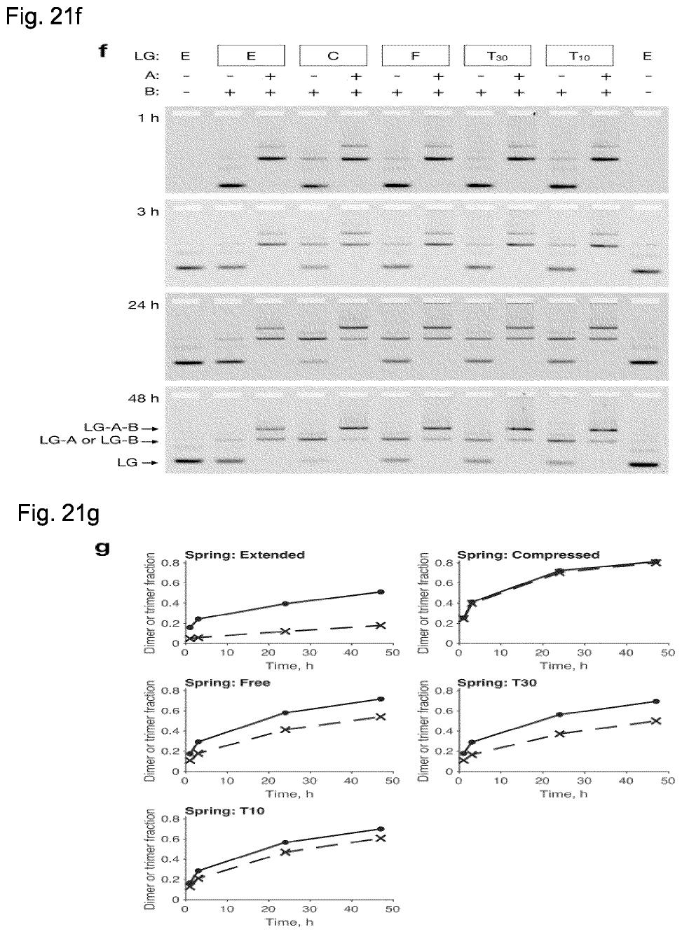

FIG. 21a depicts a schematic representation of the logic gate 1 of example 1 in the extended state (top row) and the compressed state (bottom row);

FIG. 21b depicts top and side view of logic gate 1 of example 1 average negative-staining TEM (transmission electron microscopy) particles in extended (top) and compressed (bottom) state;

FIG. 21c depicts a scheme of the conditional binding and activation of binding-site S_{B};

FIG. 21d depicts the image of an agarose gel on which logic gates or logic-gate-surface-mimic-A mixtures were electrophoresed;

FIG. 21e depicts negative-staining TEM image of logic-gate-surface-mimic-A mixture;

FIG. 21f depicts agarose gels on which incubations of logic gates with surface-mimic B (+,−) or with both surface-mimic A and B (+,+) were electrophoresed;

FIG. 21g depicts fractions of logic-gate-surface-mimic-B dimers (dashed lines) and logic-gate-surface-mimic-B-surface-mimic-A-trimers (solid lines) in incubations of logic gates with surface-mimic B or with surface-mimic B and with surface-mimic A, respectively;

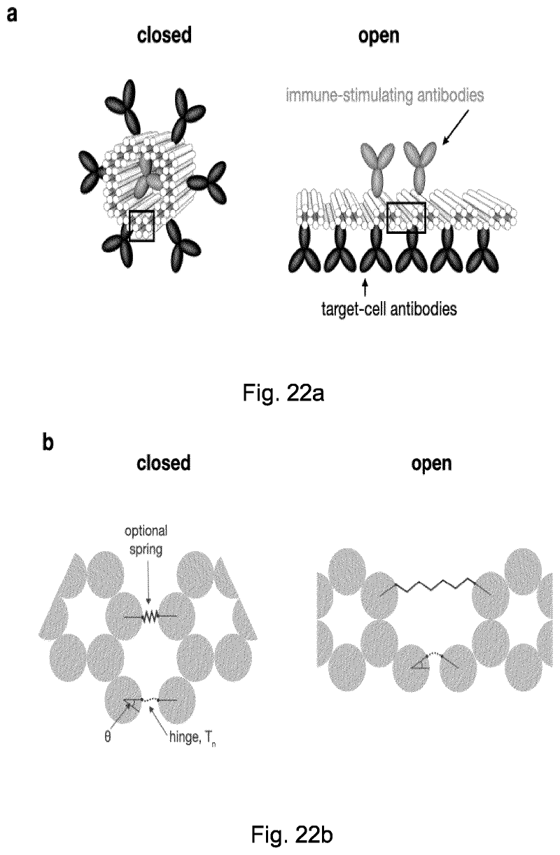

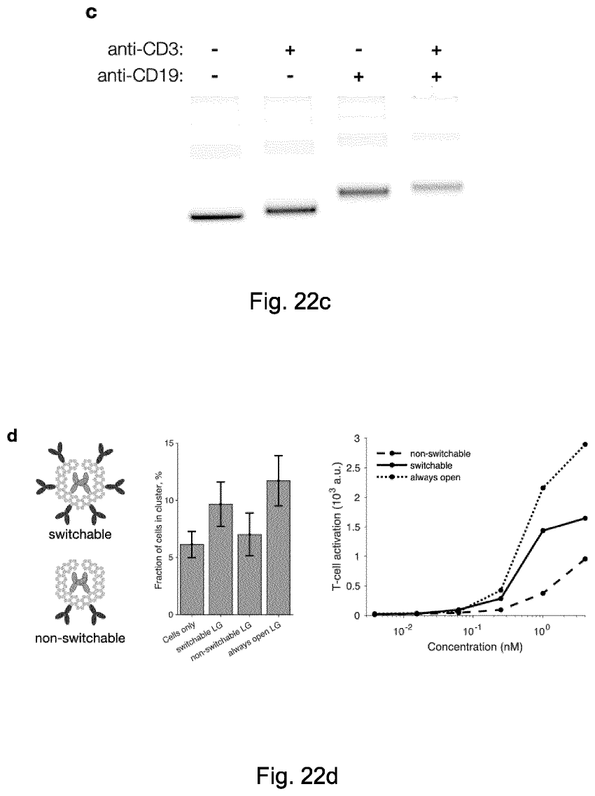

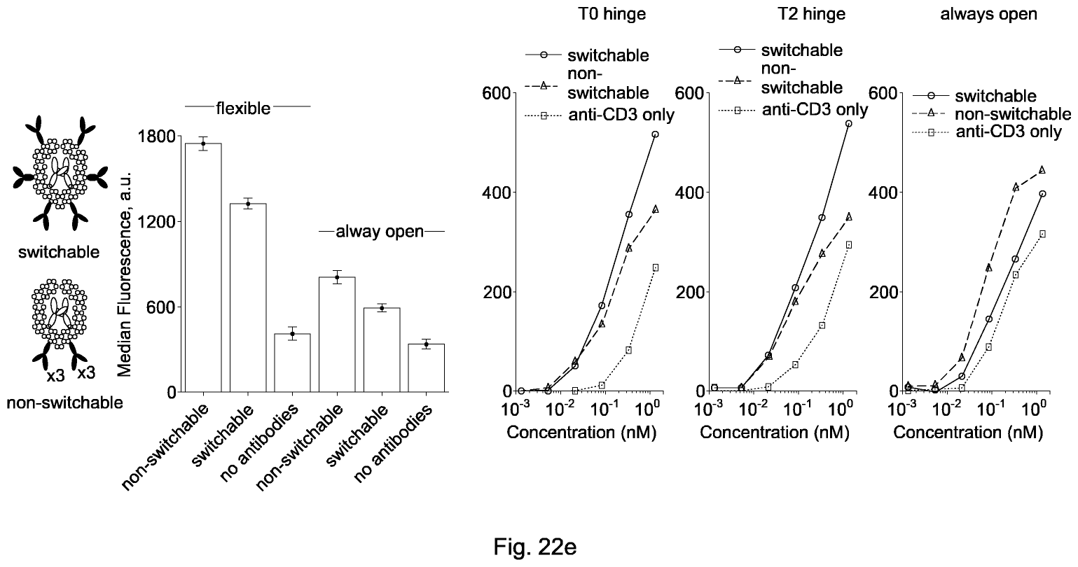

FIG. 22a depicts a schematic representation of logic gate 2 of example 2 in the closed and open state;

FIG. 22b depicts 2D-schematic representation of the hinge-region indicated in 22a) in closed and open state;

FIG. 22c depicts a 3% agarose gel on which logic gates with anti-CD3, anti-CD19, or a combination thereof was electrophoresed;

FIG. 22d depicts a schematic representation of the antibody configuration for the switchable and non-switchable variant (left panel); a diagram showing the fraction of cells in cell clusters for flexible and always open logic gates switchable or non-switchable antibody configurations (middle panel); T-cell activation signal using NALM6 cells and T Cell Activation Bioassay for logic gates variants (right panel);

FIG. 22e depicts median fluorescence from flow cytometry experiments after 1.5 h incubation (bar diagram in middle panel) and T-cell activation of different logic gate variants after 6 h (three graphs on right side panel);

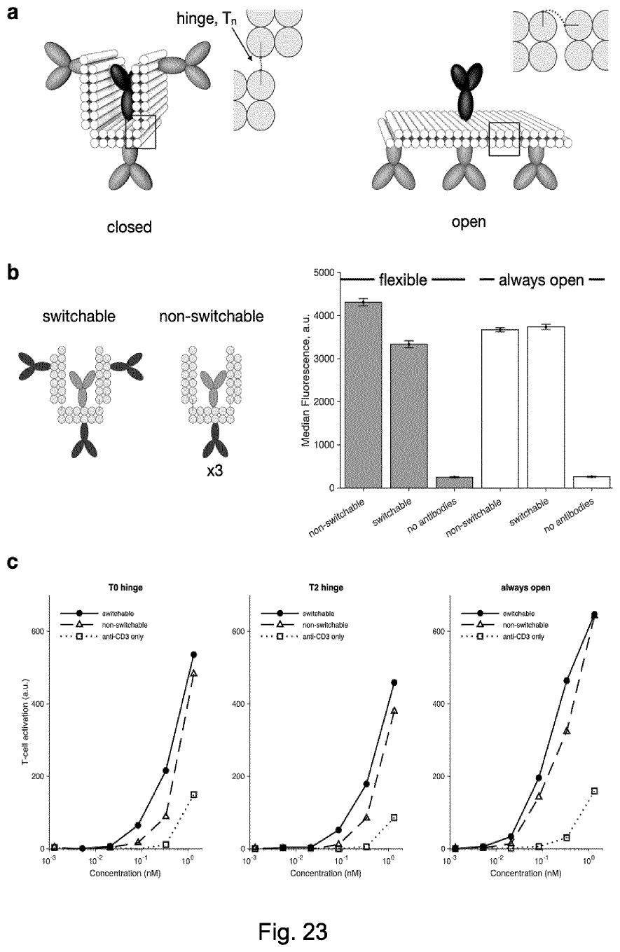

FIG. 23a depicts a schematic representation of the logic gate 3 of example 3 in the closed and open state;

FIG. 23b depicts, on the left, a schematic representation of the antibody configuration for the switchable and non-switchable variant; and on the right, median fluorescence from flow cytometry experiments after 1.5 h incubation for a flexible and always-open variant with different antibody configurations;

FIG. 23c depicts T-cell activation of different logic gate variants.

It is noted that not all the drawings carry all the reference signs. Instead, in some of the drawings, some of the reference signs have been omitted for the sake of brevity and simplicity of the illustration. Embodiments of the present invention will now be described with reference to the accompanying drawings.

In one embodiment, the invention relates to a nanostructure. Very generally, the nanostructure may comprise a first portion, a second portion, a binding site and a coupling site set comprising at least one coupling site. In examples of the present invention, the nanostructure may be a DNA origami structure, i.e., a DNA origami device, as exemplarily disclosed in U.S. Pat. No. 7,842,793 B2. However, it should be understood that the present technology is not limited thereto, and that in fact also other nanostructures may be utilized to exercise the present invention.

It will be appreciated, that the use of the singular article “a” or “the” within this document is not meant to limit the scope of the invention expect if specifically stated. That is, generally “a” may also refer to more than one. In other words, “a” can generally be read as “at least one”. For example, “a binding site” also includes a plurality of binding sites.

FIG. 1 depicts a component used in embodiments of the present technology to illustrate notations used in the present specification. More particularly, FIG. 1 depicts a nanostructure 10 that can assume different configurations A and B, which may also be referred to as a closed configuration A and an open configuration B. The nanostructure 10 may be a DNA origami nanostructure comprising two rigid domains that are flexibly connected. The two-sided arrow indicates that the nanostructure 10 may switch between the different configurations A, B and may in fact assume an equilibrium state. In such an equilibrium state, the nanostructure assumes the configurations A and B with certain probabilities.

FIG. 2 depicts further components used in embodiments of the present technology. More particularly, this Figure depicts an interaction site 1000 (such as an interaction molecule) and its interaction partner 1002, which can bind reversibly, as indicated by the two-sided arrow. Such interaction sites 1000 and interaction partners 1002 can be used as coupling sites, coupling targets, binding sites and binding targets as discussed below. Some interaction sites 1000 may bind also irreversibly to their corresponding interaction partner (not shown). That is, once bound they may generally stay in a bound state.

FIG. 3 depicts further entities 302, 304 that are used in embodiments of the present technology. More particularly, FIG. 3 (A) depicts an entity 302, 304 displaying one type of coupling targets 202, and FIG. 3 (B) depicts an entity 302, 304 displaying two types of coupling targets 202, 204. For examples, the entity 302, 304 may be a cell and the coupling targets may be molecules.

Reference will now be made to FIG. 4 schematically depicting an embodiment of a nanostructure 10 configured to assume a first configuration A and a second configuration B. The nanostructure 10 comprises a first portion 18 and a second portion 20, wherein the portions may be movably attached to each other, e.g. by a hinge-like connection. That is, the first portion 18 may move with respect to the second portion 20. For example, in the second configuration B illustrated in FIG. 1, the first portion 18 and the second portion 20 have moved with respect to each other in comparison to the first configuration A.

The nanostructure 10 further comprises a binding site 12, which may bind reversibly or irreversibly to a binding target 100 (e.g. as illustrated in FIG. 8). That is, the binding site 12 may either be configured to repeatedly bind to and detach from the binding target 100 or to generally stay bound to the binding target 100 once successfully bound. The binding site 12 and/or the binding target 100 may be a molecule. For example, reversibly binding molecules may be an antibody, an antibody fragment, a DNA strand, a biotin molecule, a streptavidin molecule, whereas irreversibly binding molecules may be a maleimide-thiol chemistry molecule, a click chemistry molecule, etc.

As discussed, in one non-limiting example, the nanostructure 10 may be a DNA origami nanostructure 10. When utilizing such a DNA origami nanostructure 10, functional sites, such as biding sites 12, or coupling sites 14 and 16 can be added to the nanostructure 10. A mechanism for this is discussed in conjunction with FIG. 5. FIG. 5, left section very conceptually depicts a DNA origami structure 10. It is intended to add a functional site 12, 14, 16 to the DNA origami structure 10. As will be understood, the DNA origami structure 10 comprises a DNA strand. The DNA strand comprises a DNA strand portion 42 to which the functional site 12, 14, 16 should be fitted. To fit the functional site 12, 14, 16 to this DNA strand portion 42, the functional site 12, 14, 16 is connected to a DNA strand portion 43 which is complementary to the DNA strand portion 42. The connection between the functional site 12, 14, 16 and the DNA strand portion 43 is provided by means of linker molecules 44. The linker reaction between the DNA strand portion 43 and the functional site 12, 14, 16 may be performed in a first, separate step by mixing the DNA strand portion 43, the linker molecules 44, and the functional sites 12, 14, 16. This reaction is typically irreversible and may be performed under specific conditions. Subsequently, the joint molecule, i.e. the compound of the DNA strand portion 43, the linker molecules 44 and the functional site 12, 14, 16, may be purified and added to a DNA origami structure 10 comprising the DNA strand portion 42 as depicted in FIG. 5, left section. The two complementary DNA portions 42, 43 may then bind to each other, and the functional site 12, 14, 16 will be coupled to the DNA origami structure 10 (see FIG. 5, right section). Alternatively, as depicted in FIG. 6, one can also provide a linker molecule 44A which is connected to a DNA origami structure 10, and provide a functional site 12, 14, 16 having a linker molecule 44B that can connect to the linker molecule 44B. Also this mechanism may enable the connection of a functional site 12, 14, 16 to a DNA origami structure.

Again with reference to FIG. 4, the binding site 12 may be more accessible for the binding target 100 in the second configuration B than in the first configuration A. In some embodiments, the binding site 12 may not be accessible for the binding target 100 in the first configuration A. That is, in these embodiments the binding site 12 may only be enabled to bind to a binding target 100 when the nanostructure 10 is not in configuration A, e.g. when the nanostructure 10 is in configuration B. However, it should be understood that the present invention is also related to embodiments realizing the opposite mechanism, i.e., where the binding site 12 is more accessible in the first configuration A than in the second configuration B.

Further, the nanostructure 10 comprises a plurality of coupling sites 14, 16, wherein the first and the second portion 18, 20 may each comprise at least one coupling site 14, 16. The coupling sites 14, 16 may either couple to coupling targets 202, 204 reversibly or irreversibly, i.e. the coupling sites 14, 16 may either bind and detach to the coupling targets 202, 204 repeatedly or generally stay bound to the coupling targets 202, 204 once successfully bound for the first time. The coupling sites 14, 16 and/or the coupling targets 100 may be molecules. For example, reversibly binding molecules may be antibodies, antibody fragments, DNA strands, biotin molecules, streptavidin molecules, whereas irreversibly binding molecules may be maleimide-thiol chemistry molecules, click chemistry molecules, etc.

In some embodiments, the coupling sites 14, 16 may be identical (i.e. configured to couple to the coupling targets 202, 204), as depicted in FIG. 7A, whereas in other embodiments the coupling sites 14, 16 may be distinct (i.e., different from one another) and configured to bind to distinct coupling targets 202, 204 (e.g. FIG. 13), i.e., to coupling targets 202, 204 having differing configurations.

The nanostructure 10 may assume the different configurations A and B depending on the binding states of the coupling sites 14, 16. Generally, coupling sites 14, 16 (or, in some embodiments also an individual coupling site) may define a coupling site set, and a coupling site sub set. In the embodiment depicted in FIG. 4 (and also FIG. 7A), the coupling sites 14, 16 define the coupling site set and the coupling site sub-set. When each of the coupling sites 14, 16 of this sub set is coupled to its respective coupling target 202, 204 (see FIG. 7A, right section), the nanostructure 10 assumes configuration B (i.e., in this embodiment, the “open” configuration where the biding site 12 is accessible). However, if none of the coupling sites 14, 16 in the coupling site set is coupled to its respective coupling target 202, 202 (see, FIG. 7A, left section), the nanostructure 10 may assume configuration A (i.e., in this embodiment, the “closed” configuration where the binding site 12 is less accessible).

Generally, it will be understood that the nanostructure 10 is configured to assume the configuration A (e.g., the closed configuration) and configuration B (e.g., the open configuration) also when the coupling sites 14, 16 are not coupled to their respective coupling targets 202, 204 (see, e.g., FIG. 7A, right section). Further, it will be understood that when the respective coupling targets 202, 204 are present in the environment of the nanostructure 10, the nanostructure 10 may assume configuration B. In this configuration, the coupling sites 14, 16 of the nanostructure 10 may then couple to their respective coupling targets 202, 204. Once the coupling sites 14, 16 are coupled to their respective coupling targets 202, 204, this may render configuration B more likely than would be the case without such a coupling. Thus, when the coupling site 14, 16 are coupled, the nanostructure 10 may be more likely to assume configuration B than would be the case without such a coupling.

In other words, to couple to the coupling targets 202, 204, the nanostructure 10 is in the second configuration. More particularly, the nanostructure 10 may randomly move into the second configuration (usually, this happens rarely). Once this happens and in case the coupling targets 202, 204 are present, the coupling sites 14, 16 can all be bound at the same time. Once this happens, the nanostructure 10 is unlikely to leave the second configuration anymore, thus increasing the time spent in the second configuration.

That is, in this embodiment, the nanostructure 10 may be configured to assume the first configuration A when none of the coupling sites 14, 16 is coupled to its respective coupling target 202, 204. It will be understood that the nanostructure 10 may still be able to assume a different configuration, e.g., the second configuration B. However, when none of the coupling sites 14, 16 is coupled to its respective coupling target 202, 204 (i.e., when coupling site 14 and coupling site 16 is not coupled to its respective coupling target), the nanostructure 10 may be configured to assume the first configuration A. In some embodiments, the nanostructure 10 may be configured to assume the first configuration A with a higher probability than the second configuration B when none of the coupling sites 14, 16 is coupled to its respective coupling target.

Further the nanostructure 10 may be configured to assume the second configuration B when each coupling site 14, 16 of a subset of the coupling site set is coupled to its respective coupling target 202, 204. It will be understood that the nanostructure 10 may also assume the second configuration B in cases where not all coupling sites 14, 16 of the subset are coupled to their respective coupling targets 202, 204 (see FIG. 4, right section).

With reference to FIGS. 7A and 7B, the coupling sites 14, 16 may couple to coupling targets 202, 204 comprised by a single entity 302, also referred to as first entity 302, (see FIG. 7A) or to coupling targets 202, 204 comprised two entities 302, 304, also referred to as first entity 302 and second entity 304 (see FIG. 7B), wherein the two entities 302, 304 may in some embodiments be two distinct entities 302, 304. In some embodiments the entities 302, 304 may be cells, for example T-cells or cancer cells. Typically, an entity 302, 304 may comprise a plurality of coupling targets 202, 204 and/or a plurality of binding targets 100.

Generally, it will be understood that the nanostructure 10 can assume equilibrium states between the first configuration A and the second configuration B (see FIG. 4). Again, the binding site 12 may be more accessible in one configuration compared to the other configuration, for example, the binding site 12 may be more accessible in in the second configuration B than in the first configuration A. The nanostructure 10 may assume a first equilibrium state between the first configuration A and the second configuration B when the respective coupling targets 202, 204 are absent, i.e., when none of the coupling sites 14, 16 can couple to a respective coupling target 202, 204. Such an equilibrium state is illustrated by the two-sided arrow in FIG. 4. As a mere example, it may be possible that the first equilibrium state that the nanostructure 10 assumes in FIG. 4 is such that the nanostructure spends 90% in configuration A and 10% in configuration B. In other words, in the first equilibrium state, the nanostructure 10 being is in configuration A with a probability of 90% and in configuration B with a probability of 10%.

Further, the nanostructure 10 may assume a second equilibrium state between the first configuration A and the second configuration B when all of the respective coupling sites 14, 16 of a subset of the set of coupling sites can couple to respective coupling targets 202, 204 (see, e.g., FIG. 7A). The probability that the nanostructure 10 assumes the second configuration B may be higher in the second equilibrium state of the nanostructure 10 than in the first equilibrium state. Again, the second equilibrium state between the different configurations A and B is illustrated in FIG. 7A by means of the two-sided arrow. When the nanostructure 10 is in an environment where the coupling sites 14, 16, can couple to respective coupling targets 202, 204 (see FIG. 7A, 7B), configuration B of the nanostructure 10 is more favourable than in the situation where such a coupling is not possible. Thus, in such an environment, the nanostructure 10 has a higher probability of assuming the second configuration B. As a mere example, in the second equilibrium state, the nanostructure may assume the second configuration with a probability of 90% and the first configuration with a probability of 10%.

It will be understood that the nanostructure may in some embodiments always be in configuration B once the subset of coupling sites 14, 16 is coupled to the respective coupling targets 202, 204. For example, in embodiments where the coupling sites 14, 16 may couple irreversibly to the coupling targets 202, 204.

As discussed, in the first equilibrium state the nanostructure 10 may assume the first configuration A with a probability of 0.9 and the second configuration B with a probability of 0.1. In such a case it may be unlikely that the binding site 12 can bind to its binding target 100. Therefore, the first equilibrium state may in such cases also be referred to as slow binding state. In contrast, in the second equilibrium state the nanostructure 10 may assume the first configuration A with a probability of 0.1 and the second configuration B with a probability of 0.9. Thus, the second equilibrium state may in such cases also be referred to as fast binding state.

It will be understood that the above is merely an example and that the first and second equilibrium state may assume the first and second configuration A, B with different probabilities. In general, the second configuration B may be assumed with a higher probability in the second equilibrium state than in the first equilibrium state.

As described above, in some embodiments the binding site 12 may be more accessible in the second configuration B than in the first configuration A. In some embodiments, the binding site 12 may not be accessible in the first configuration A. However, if the nanostructure 10 assumes the second configuration B, a binding target 100 may bind to the binding site 12 of the nanostructure 10, as depicted in FIG. 8. In other words, the binding of the binding site 12 to a binding target 100 may mainly depend on the configuration the nanostructure 10 assumes.

Again, when the nanostructure 10 is in an environment where its coupling sites 14, 16 can both couple to their respective coupling targets 202, 204 (such as depicted in FIGS. 7A and 7B), the nanostructure 10 is more likely to assume the second configuration B (i.e., in the present example, the more accessible configuration).

Thus, if a binding target 100 is present (see FIG. 8), it is more likely that such a binding target 100 binds to a nanostructure 10 where both the coupling sites 14, 16, are coupled to respective coupling targets 202, 204, i.e. all coupling sites of the subset of the coupling site set, (see FIG. 8, lower section) than that such a binding target 100 binds to a nanostructure 100 where none (or only one) of the coupling sites 14, 16, is coupled to its respective coupling target 202 (see FIG. 8, upper section). In other words, both coupling sites 14, 16 being coupled to their respective coupling targets 202, 204 may “activate” the binding site 12, as binding the binding site 12 to a binding target 100 is more likely when both coupling sites 14, 16 are coupled to their coupling targets.

In the embodiment depicted in FIG. 8, the coupling sites 14, 16 of the nanostructure 10 are configured to couple to coupling targets 202, 204 located on a single entity 302, e.g., a single cell. However, this is not necessary, as already indicated by FIG. 7B. With regard to FIG. 7B, corresponding considerations as presented in conjunction with FIG. 8 apply, the only difference being that in the configurations depicted in the lower section of FIG. 8, the coupling sites 202 of the nanostructure 10 are not coupled to coupling targets 202, 204 comprised by a single entity, but to coupling targets 202, 204 comprised by different entities (as in FIG. 7B, right section).

It will be understood that the above is merely an example and that in other embodiments the binding site 12 may be more accessible in configuration A than in configuration B. Such an embodiment is shown in FIGS. 9a, 9b and 10. FIG. 9a depicts a nanostructure that is configured to assume two configurations A and B. It will be appreciated that the binding site 12 is more accessible in the first configuration A than in the second configuration B. Again, the double headed arrow illustrates that there may be an equilibrium of the nanostructure 10 assuming the first configuration A and the second configuration B. FIG. 9b depicts the nanostructure 10 being in the first configuration and a binding target 100 being bound to the binding site 12.

Again, the nanostructure 10 comprises coupling sites 14, 16, forming a coupling site set and a sub-set (the coupling site set and the sub-set being equal in this embodiment). As in the other embodiments, each coupling site 14, 16 is configured to couple to a coupling target 202, 204. FIG. 10 depicts an equilibrium (again illustrated by a two-headed arrow) state between the two configurations A and B. It will be appreciated that when the entities 302, 304 comprising the coupling targets 202, 204 are present in the environment of the nanostructure 10, the nanostructure 10 is more likely to assume the second configuration B than when such entities 302, 304 with the coupling targets 202, 204 are not present. In other words, when the entities 302, 304 are present, the equilibrium is “biased” more towards the configuration B than when such entities 302, 304 are not present. Further, again with reference to FIG. 9b, it will be understood that when such entities 302, 304 are present, it will be less likely that the binding target 100 binds to the binding site 12 (as the nanostructure 10 will spend a smaller amount of time in the “accessible” configuration A).

Again with general reference to FIGS. 9a, 9b and 10, the nanostructure 10 comprises a first portion 18 and a second portion 20, wherein each portion comprises a coupling site 14, 16. Further, the first portion 18 comprises a binding target 12. However, in contrast to previously shown embodiments of the present invention (e.g. FIG. 4), the binding site 12 is more accessible in configuration A. That is, the binding site may be more accessible if none of the coupling sites 14, 16 is coupled to a respective coupling target 204, 206 (left side of the Figure). In other words, through coupling of the coupling sites 14, 16 of the subset of the coupling site set (here the subset may comprise for example both coupling sites 14, 16) to coupling targets 202, 204 comprised by two entities 302, 304 the access to the binding site may be impeded or prevented.

With reference to FIG. 9b the binding of the binding site 12 to a binding target 100 is shown for a nanostructure where the binding site 12 is more accessible in the first configuration A than in the second configuration B. In some embodiments, the binding site 12 may not be accessible in the second configuration B.

It will be understood that the considerations discussed in conjunction with the other nanostructures may also apply accordingly to such a nanostructure 10. For example, the nanostructure depicted in FIG. 9a to 10 may also comprise distinct coupling targets as discussed with reference to FIG. 13.

With reference to FIG. 11A a nanostructure 10 is shown that comprises similar features to the nanostructure 10 discussed previously with reference to for example FIGS. 4 and 8. However, in the embodiment shown in FIG. 11A the coupling site set of the nanostructure 10 comprises three coupling sites 14A, 14B, 16. In the depicted embodiment two coupling sites 14A, 14B are attached to the first portion 18 of the nanostructure 10 and one coupling site 16 is attached to the second portion 20. It will be understood that this is merely an example and that generally a plurality of coupling sites 14A, 14B, 16 may be attached to either the first portion 18 or the second portion 20.

The binding site 12 is more accessible in the second configuration B than in the first configuration A and FIG. 11B depicts the nanostructure 10 with a bound binding target 100. Again, such binding may be reversible or irreversible depending on the type of interaction.

Generally, a nanostructure 10 may assume the second configuration when each coupling site 14, 16 of a subset of the coupling site set is coupled to its respective coupling target 202, 204. FIG. 12A depicts two realisations B′ and B″ of the second configuration B, wherein the coupling sites comprised by a subset are coupled to their respective coupling targets comprised by two entities 302, 304 and wherein the subset of coupling sites is different. For the first realisation B′ the subset comprises one coupling site 14A attached to the first portion 18 and one coupling site 16 attached to the second portion 20 and for the second realisation B″ the subset comprises another coupling site 14B attached to the first portion 18 and the same coupling site 16 attached to the second portion 20. That is, for a given embodiment of a nanostructure 10 the subset of the coupling site may be defined to comprise a combination of coupling sites 14A, 14B, 16. For example, the subset may comprise one coupling site 14A, 14B, 16 per portion, i.e. for each portion 18, 20 the subset may comprise a coupling site 14A, 14B, 16 attached to the respective portion 18, 20.

With reference to FIG. 12B the nanostructure 10 is shown with a bound binding site 12. In the depicted embodiment, the binding site 12 is more accessible in the second configuration B, B′, B″ than in the first configuration A. That is, the binding site 12 may be more accessible to a binding target 100 when the nanostructure 10 is in the second configuration B, B′, B″. The accessibility may generally not depend on the realisation of the second configuration B, i.e. it may generally be the same for the all realisations B′, B″ of the second configuration.

Throughout the embodiments depicted in FIGS. 4, 7A, 7B, 8, 9, 10, 11A, 11B, 12A and 12B, the coupling sites 14, 16 are of the same configuration, i.e., they are configured to couple to coupling targets 202, 204 of the same design.

However, as already mentioned above, this is not necessary. In some embodiments of the present invention the coupling sites 14, 16 may bind to distinct coupling targets 202, 204 (i.e., to coupling targets 202, 204 with differing configuration), as schematically depicted in FIG. 13. That is, each coupling site 14, 16 may only couple to the respective coupling targets 202, 204.

Generally (i.e., independent of whether or not the coupling sites 14, 16 are of the same or of differing configurations), the respective coupling targets 202, 204 may be comprised by a single entity 302 (see, e.g., FIGS. 7A and 8) or by a first and a second entity 302, 304 (see FIGS. 7B and 13, lower section). Generally, a single entity may comprise a plurality of coupling targets 202, 204.

In some embodiments, a single entity 302, 304 may only comprise the respective coupling targets 202 or 204 for one of the distinct coupling sites 14, 16, wherein in other embodiments a single entity 302, 304 may comprise a plurality of respective coupling sites 202, 204—see FIG. 3 in this regard.

That is, generally, the present technology is applicable in different scenarios. The scenarios include a bivalent nanostructure 10, i.e., a nanostructure 10 having one type of coupling sites 14, 16, and one type of binding site 12, which coupling sites 14, 16 couple to coupling targets 202, 204 of a single entity 302, i.e., to a single object. This case may be referred to as the bivalent (one type of binding site, one type of coupling site) one object case, and is depicted in FIGS. 4, 7A, and 8. Further, correspondingly, there may be a bivalent two object case (one type of biding site, one type of coupling site, coupling to two objects) as depicted in FIG. 7B; a trivalent one object case (one type of binding site, two types of coupling site, coupling to one object) as depicted in FIG. 13, upper section; and a trivalent two object case (one type of binding site, two types of coupling site, coupling to 2 objects) as depicted in FIG. 13, lower section. Further any multivalent nanostructure 10 may be possible, that is a structure with a plurality of different coupling sites 14,16 and/or binding sites 12 as discussed below with reference to FIG. 14A and following. For all of these cases, corresponding considerations apply.

In particular, it should be understood that the accessibility of a binding site 12 of the nanostructure 10 may be different when the coupling sites 14, 16 of the subset of the coupling site set are coupled to their respective coupling targets 202, 204 compared to the case that none of the coupling sites 14, 16 are coupled to their respective coupling target.

As already mentioned, the present invention also comprises multivalent nanostructures 10. That is, a nanostructure 10 may comprise a plurality of types of coupling sites 14, 16 and/or binding sites 12. An exemplary embodiment of a multivalent nanostructure 10 is depicted in FIGS. 14A to 15B.

The depicted nanostructure 10 comprises a coupling site set comprising four coupling sites 14 A, 14B, 16A, 16B, wherein each coupling site may be of a different configuration. That is, the coupling sites 14 A, 14B, 16A, 16B, may each be distinct from each other. However as previously discussed at least a portion of the coupling sites 14 A, 14B, 16A, 16B, may also be of the same configuration, e.g. the coupling sites attached to the same portion 18, 20 may be identical. Further, the nanostructure 10 may comprise a plurality of binding sites 12A, 12B, wherein the binding sites may be of identical or different configuration. That is, in some embodiments all binding sites may be distinct from each other, whereas in other embodiments at least a portion of the plurality of binding sites may of the same type, i.e. the same configuration.

The nanostructure may be configured assume the first configuration if none of the coupling sites 14 A, 14B, 16A, 16B, is coupled to the respective coupling target (see FIG. 14A, most left). Further, the nanostructure 10 may also be configured to assume the first configuration A when not all coupling sites 14 A, 14B, 16A, 16B of a subset of the coupling site set are coupled to the respective coupling targets 202, 204. That is, for example, in the depicted embodiment, this may be the case if only one coupling target 14 A, 14B, 16A, 16B, or two coupling targets attached to the same portion 18, 20 may be coupled to their respective coupling targets 202, 204 (some examples are depicted in FIG. 14 A).

It will be understood that albeit being configured to assume the first configuration A when none of the coupling sites 14 A, 14B, 16A, 16B, is coupled to its respective coupling target, the nanostructure 10 may still assume another configuration, such as the second configuration B as depicted on the right hand side of FIG. 14A. Generally, it should be understood that the requirement that the nanostructure 10 is configured to assume a particular configuration (in a certain coupling state of the coupling sites) does not mean that the nanostructure 10 is configured to only assume this configuration.

Furthermore, the nanostructure 10 may be configured to assume a first and second equilibrium state between the first configuration A and the second configuration B. That is, the nanostructure 10 may assume the first configuration A and the second configuration B each with a certain probability as indicated by the two-sided arrow. The probability that the nanostructure 10 assumes the second configuration B may be higher in the second equilibrium state of the nanostructure 10 than in the first equilibrium state. Further, the nanostructure 10 may be configured to assume the second equilibrium state when all coupling sites 14A, 14B, 16A, 16B of a subset of the coupling site set are coupled to the respective coupling targets 202, 204, whereas the nanostructure may be configured to assume the first equilibrium state when none of the coupling sites 14A, 14B, 16A, 16B, of the coupling site set are coupled to their respective coupling targets 202, 204.

As previously discussed, the accessibility of the binding sites 12A, 12B may depend on the configuration assumed by the nanostructure 10. That is, the accessibility of the binding sites 12A, 12B may be different when the nanostructure 10 is in the first configuration A compared to when the nanostructure 10 is in the second configuration B. In the depicted embodiment, the binding sites 12A, 12B may be more accessible when the nanostructure assumes the second configuration B. Moreover, in some embodiments, the binding sites 12A, 12B may not be accessible in the first configuration A.

Whenever the binding sites 12A, 12B are accessible a corresponding binding target 100A, 100B may bind to it as depicted in FIG. 14 B. That is, if a binding target 100A, 100B is present and the binding sites 12A, 12B of the nanostructure 10 are accessible, the binding targets 100A, 100B may bind to the binding sites 12A, 12B. Consequently, if a binding site 12A, 12B is more accessible, the probability of binding to a binding target 100A, 100B may be higher.

Further all previous considerations may apply, particularly the binding of binding sites 12A, 12B and the coupling of coupling sites 14A, 14B, 16A, 16B may be reversible or irreversible depending on the type of interaction site 1000. That is, depending on the configuration (or type) of each coupling or binding site, the corresponding coupling or binding process may be reversible or irreversible. For example, a nanostructure may comprise a coupling site 14A, 14B, 16A, 16B that binds reversibly as well as another coupling site 14A, 14B, 16A, 16B that binds irreversibly.

With reference to FIG. 15A the coupling sites 14A, 14B, 16A, 16B may couple to coupling targets 202A, 202B, 204A, 204B comprised by two entities 302, 304. It will be appreciated, that in other embodiments of the present technology all the coupling targets 202A, 202B, 204A, 204B may also be comprised by a single entity 302,304.

As mentioned before, the nanostructure 10 may be configured to assume the second configuration B when each coupling site 14A, 14B, 16A, 16B of a subset of the coupling site set is coupled to the respective coupling target 202A, 202B, 204A, 204B. A subset may for example comprise one coupling site 14A, 14B, 16A, 16B for each portion 18, 20, wherein the coupling site 14A, 14B, 16A, 16B is attached to said portion 18, 20 of the nanostructure 10. Examples where all coupling sites 14A, 14B, 16A, 16B of such a subset are coupled to the respective coupling targets 202A, 202B, 204A, 204B are shown to the right of the two-sided arrow in FIG. 15A. It will be appreciated, that more coupling sites 14A, 14B, 16A, 16B than comprised by the subset may couple to their respective coupling targets as for example in the right most example in FIG. 15A, where three coupling sites 14B, 16A, 16B are coupled to their respective coupling targets 202B, 204A, 204B.

Again, if binding targets 100A, 100B are present and the binding sites 12A, 12B are accessible the binding targets 100A, 100B may bind to the binding sites 12A, 12B as depicted in FIG. 15B. Previous considerations may apply.

In the following an example of a nanostructure 10 comprising two distinct coupling sites 14, 16 is presented, first conceptually and subsequently with reference to an exemplary implementation.

FIG. 16A depicts a nanostructure 10 comprising a binding site 12 and two coupling sites 14, 16, wherein the coupling sites 14, 16 are different to each other. In other words, the coupling sites 14, 16 are configured to couple to distinct coupling targets 202, 204.

The nanostructure 10 may be in a solution or generally in an environment with the first entity 302 comprising a plurality of binding targets 100 and a plurality of coupling targets 202, wherein the coupling targets 202 may only couple to one of the distinct coupling sites 14, 16.

That is, one coupling site 14 of the nanostructure 10 may couple to a coupling target 202 comprised by the first entity 302. Without other entities being present, the nanostructure 10 may be in a first equilibrium state in which the nanostructure 10 is predominantly, or in some embodiments essentially solely, in the first configuration A and therefore the coupling site 14 only bind solely to the coupling targets comprised by the first entity 302, without the other coupling site 16 coupling to its respective coupling target. In other words, binding of the binding site 12 to the binding target 100 may be (at least mostly) inhibited by the conformation of the nanostructure 10.

However, as depicted in FIG. 16B, the solution or environment may further comprise a second entity 304 comprising a plurality of coupling targets 204 distinct from the coupling targets 202 comprised by the first entity 302 and configured to couple to the other coupling site 16. In such an environment, the nanostructure 10 may couple to both entities 302, 304, i.e. the coupling site 14 couple to the coupling targets 202 and the coupling site 16 may couple to coupling target 204, wherein the coupling targets 202, 204 are each comprised by a different entity 302, 304. In such a coupled state, the nanostructure may comprise a second configuration B. In this configuration also the binding site 12 may bind to the corresponding binding target 100. Therefore, in a solution or environment comprising both entities 302, 304, the nanostructure 10 may be more likely to assume a configuration allowing the binding site 12 to bind to binding target 100. Again, in principle, the configuration allowing the binding site 12 to bind to a binding target 100 may also be assumed when the second entity 304 is not present. However, when the second entity 304 is not present, the nanostructure 10 may assume an equilibrium state making it unlikely that the nanostructure 10 is in such a configuration. Conversely, when the second entity 304 is present, it may be more likely that the nanostructure 10 assumes the configuration allowing the binding site 12 to bind to the binding target 100. That is, when the second entity is present, the nanostructure 10 may be in an equilibrium state rendering this configuration more likely than in the previously discussed equilibrium state.

Such a nanostructure 10 may be beneficial as it may bring two entities into close proximity through coupling of the coupling sites 12, 14 to the respective coupling targets 202, 204 and subsequently for example activate a process within an entity 302, 304 by further binding the binding site 12 to the corresponding binding target 100.

Further still, it will be understood that such a nanostructure 10 may be “inactive” unless activated by the presence of both the first entity 302 comprising the first coupling target 202 and the second entity 304 comprising the second coupling target 204. When such first entities 302 and second entities 304 are present, the nanostructure 10 is “activated” and its binding site 12 becomes accessible. Once accessible, the binding site 12 may bind to a binding target 100. In the example discussed in conjunction with FIG. 16A and FIG. 16B, the binding target 100 is comprised by the first entity 302 also comprising the first coupling target 202.

An exemplary embodiment will now be discussed with reference to FIGS. 16C, 17A, and 17B. In this embodiment, a nanostructure 10 comprises two distinct coupling sites 14, 16. One coupling site 14 may be configured to couple to a CD3 antigen, and may therefore also be referred to as a CD3-antigen-coupling site or CD3 antigen-coupling fragment. The other coupling site 16 may be configured to couple to an epithelial cell adhesion molecule (EpCAM) and may therefore be referred to as an EpCAM-coupling site. Further, the binding site 12 may be configured to bind to a CD28 protein and may therefore also be referred to as a CD28-binding site or simply anti-CD28.

For such an embodiment, the first entity 302 may be a T-cell 302 comprising CD3 antigens 202 as coupling targets 202, and CD28 proteins as binding targets 100, and the second entity 304 may be a tumor cell 304 expressing EpCAM as the coupling target 204 (see FIG. 10C).

Thus, in a solution or environment such an embodiment may bring a tumor cell 304 and a T-cell 302 in close proximity by coupling to a CD3 antigen 202 on the T-cell 302 and by coupling to an EpCAM 204 on the tumor cell 304. Subsequently the T-cell 302 may be at least further activated by the binding site 12 of the nanostructure 10 binding to the CD28 protein 100. Thus, a solution or composition comprising nanostructures 10 may be used as a medicament.

With reference to FIG. 17A an exemplary implementation of such a nanostructure 10 is shown. The nanostructure 10 may comprise a first and a second portion 18, 20 with a generally cuboid shape, i.e. the shape of a rectangular box. Moreover, the two portions 18, 20 may be identical with regard to their shape (though this is not a necessity), which may be advantageous in design and fabrication of such a nanostructure 10.

The outer dimensions of each portion 18, 20 may be determined by a length L, a breadth B and a height H, wherein typically L>B>H. The length L may be smaller than 1000 nm, preferably smaller than 500 nm, such as 100 nm.

The two portions 18, 20 may be movably connected along an edge 32, wherein the edge is of dimension B, i.e. it is an edge of a short side of the rectangular portions 18, 20.

Further, each portion 18, 20 comprises a coupling site 14, 16, e.g. a CD3 antigen-coupling site 14 and an EpCAM-coupling site 16. The coupling sites 14, 16 may each be located on a corresponding small end surface 34, 37 of the two portions 18, 20, i.e. a surface with dimensions B×H, wherein the surface is located opposite to the side where the two portions 18, 20 are connected, i.e. opposite to the edge 32. That is, the two coupling sites 14, 16 may be adjacent to each other when the nanostructure 10 assumes a closed configuration, i.e. when the two portions 18, 20 are in contact with the two outer surfaces 35, 38 with dimension L×B and comprising the edge 32 at which the two portions 18, 20 are connected. In this example the closed configuration may correspond to the first configuration A, where the binding site 12 is not accessible.

The two portions 18, 20 may each comprise a cavity 36, 39 configured to form a chamber within the two portions 18, 20 when they assume the closed configuration.

The binding site 12 may be located within the cavity 36 of the portion 18 comprising the coupling target 14. The binding site 12 may be an CD28-binding site 12. The binding site 12 may be attached to the portion 18 by means of a rod 40. That is, once the nanostructure 10 may assume an open configuration, i.e. when the two portions are in a position where the two cavities 36, 39 are exposed to the surrounding of the nanostructure 10, the binding site 12 may leave the cavity 36 and preferably bind to a binding target 100 in the vicinity of the nanostructure 10.

It is noted, that in the context of this document a rod 40 may be any type of flexible link between a portion of the cavity 36 and at least one binding site 12.

Reference will now be made to the illustration in FIG. 17B. This series of Figures depicts a mode of action of the nanostructure 10 depicted in FIG. 17A. In a solution or composition containing a nanostructure 10 as described above as well as T-cells 302 and tumor cells 304, the nanostructure 10 may generally assume the first configuration A, i.e. the nanostructure 10 may be closed (or, more generally, assume the configuration where the binding site 12 is not accessible). In a first step, the coupling site 16 may bind to a corresponding coupling target 204, i.e. EpCAM 204, on a tumor cell 304. Subsequently the coupling site 14 may bind to its respective coupling target 202, i.e. CD3 202, on a T-cell 302. At this point the nanostructure 10 may assume the second configuration B, which in this example corresponds to the open configuration (where the binding site 12 is accessible). Once the nanostructure 10 is open, the binding site 12 may leave the cavity 36 and bind to a close-by binding site 100, i.e. CD28, comprised by the T-cell 302. This process may activate the T-cell 302 and thus such a solution or composition comprising such nanostructures may be used in the treatment of cancer.

That is, in the implementation depicted in FIG. 17B, the nanostructure 10 can be used to selectively activate the T-cells. More particularly, the nanostructure 10 is more likely to assume the open, or “activated” configuration in a second equilibrium state, which the nanostructure 10 assumes when both the T-cells 302 (displaying CD3) and the tumor cells 304 (displaying EpCAM) are present. In such a scenario (T-cells 302 and tumor cells 304 present), the nanostructure 10 assumes the “activated” configuration with a higher probability as in the case where at least one of the T-cells 302 and the tumor cells 304 is not present. This then leads to the binding site 12 binding to its respective binding target (i.e., here: the CD28 of the T-cells), and thus further activating the T-cells.

Other implementations may for example allow for targeted drug delivery.

FIG. 18A depicts an implementation of a nanostructure 10. The first portion 18 of the nanostructure 10 may be a rod 18A comprising recesses 181, 182 at each end in the longitudinal direction, i.e. the direction along the maximum extend of the rod 18A. The two recesses 181, 182 may be oriented perpendicular to the longitudinal axis and facing in opposite directions, i.e. bottom surfaces 1811, 1821 of the two recesses 181, 182 may lie in parallel planes.

Further, the rod 18A may be configured to rotate (with respect to the second portion) around a rotational axis 183 running through the centre of the rod 18A in a direction perpendicular to the longitudinal axis and perpendicular to the bottom surfaces 1811, 1821 of the two recesses 181, 182.

Additionally, one recess 181 may comprise a coupling site 16, whereas the other recess 182 may comprise a binding site 12.

The second portion 20 of the nanostructure 10 may be a hollow disc 20A, comprising two outer discs 21, 23. Each of the two outer discs 21, 23 comprises a disc recess 22, 24 configured to provide access to the end of the rod 18 when assembled. More precisely the disc recesses 22, 24 are configured to provide access to the coupling site 16 and/or the binding site 12 that are attached within the two recesses 181, 182 of the rod 18A.

The two outer discs 21, 23 are connected through at least one connection structure 25 such that a hollow disc is formed, configured to take up the rod 18A. The outer discs 21, 23 may be combined in a way that the two disc recesses 22, 24 are rotated by 180° with respect to each other. In other words, the disc recesses 22, 24 are arranged such that the two recesses 181, 182 of the rod 18A may be accessible at the same time.

The rod 18A may be held in the centre of the hollow disc 20A. That is, the rod 18A may rotate freely within the hollow disc 20A, and the binding site 12 and the coupling site 16 may be accessible only when the rod 18A is in a position where the two recesses 181, 182 line up with the corresponding disc recesses 22, 24 (second configuration B).

Further, the outer disc 23 may comprise a coupling site 14 located at the outer rim and adjacent to the disc recess 24 that is configured to reveal the coupling site 16 comprised by the recess 181 of the rod 18A.

The assembled nanostructure 10 is shown in FIG. 18B in a position where the recesses of the rod 181, 182 line up with the two disc recesses 22, 24, such that the coupling site 14, 16 and the binding site 12 are accessible.

With reference to FIG. 19, the rod 18A may generally move within the hollow disc 20A. That is, overall the coupling site 16 and the binding site 12 may not be accessible for most of the time, i.e. the nanostructure may be in a first configuration A (or first configurations) for most of the time. Again, the two-sided arrow in FIG. 19 indicates that there is an equilibrium state between the configurations A and B, and it may be understood that normally (i.e., without any other entity being present), the nanostructure 10 may assume the configuration A, where the binding site 12 is not accessible with a higher likelihood than configuration B, where the binding site 12 is accessible. As in the previously discussed embodiments, when a binding target is present in such an equilibrium state, the binding target will only bind slowly to the binding site 12 (as the binding site is not accessible most of the time).

FIG. 20 depicts the nanostructure of FIG. 19 in a situation where also an entity 302 comprising aptly located binding targets 202, 204 is present. In such a situation, the coupling site 14 may couple to the respective coupling target 202 (see FIG. 20, right section). In case the entity 302 further comprises the coupling target 204 corresponding to the coupling site 16 comprised by the rod 18A at a suitable location with respect to the coupling target 202 (see again FIG. 20, right section), the coupling site 16 may couple to this coupling target 204. Generally, the chances of said coupling site 16 coupling to the coupling target 204 may significantly increase once the coupling site 14 is bound to the coupling target 202, i.e. the nanostructure 10 may then assume the second configuration B. That is, once the entity 302 is close to the disc recess 24 due to the binding of coupling site 14 and coupling target 202, the coupling target 204 may be present and there is a high chance that the coupling site 16 may bind to it as soon as the random movement of the rod 18 aligns it with the disc recess 24.

Further, once the coupling site 16 is bound to its coupling target 204, the binding site 12 is aligned with the other disc recess 22 and therefore accessible for binding to a corresponding binding target 100 (second configuration B). That is, the probability of binding to a binding target 100 is significantly higher compared to a state where the coupling site 16 is not bound to a coupling target (first configuration A).

With exemplary reference to the embodiment depicted in FIGS. 18A to 20, it should also be understood that the coupling site 14 is not necessary. In other words, this coupling site can also be omitted. Again, it will be understood that the rod 18A (see FIG. 18B) may rotate within disc 20A. Thus, when an end of the rod 18A comprising the coupling site 16 is aligned with disc recess 24, the coupling site 16 may couple to a coupling target (also in an embodiment not comprising coupling site 14). It will be understood that the binding site 12 will then also be aligned with the respective disc recess 22, and will thus be accessible. Thus, such an embodiment can also be realized with one coupling site 16 only. That is, the accessibility of the binding site 12 is regulated by the coupling state of one coupling site 16 only.

Such a three-dimensional nanostructure 10 may be realized using DNA origami, i.e. by combining scaffolding strands and staple stands to form the required portions and the overall device. Such designs may for example be performed using software such as caDNAno. That is, a nanostructure 10 comprising multiple portions 18,20 may in some embodiments be made out of one scaffolding strand, whereas in other embodiments portions of a nanostructure 10 may be constructed utilizing a plurality of scaffolding strands.

Again, also with regard to the embodiment depicted in FIGS. 7A, 7B, and 8, it will be understood that the nanostructure 10 may assume different configurations, and that there may be different equilibrium states between the different configurations. Again, the nanostructure 10 may assume the different equilibrium states depending on other structures, such as an entity 302 comprising coupling targets 202, 204 at suitable locations. More particularly, when such an entity 302 is present, the nanostructure 10 may assume an equilibrium state in which it assumes a position where the binding site 12 is accessible with a higher probability than in the equilibrium state when such an entity is not present. Again, in simple words, also the nanostructure 10 depicted in FIGS. 7A, 7B, and 8 may thus be “activated” for binding to a binding target when an entity 302 as discussed is present.

Whenever a relative term, such as “about”, “substantially” or “approximately” is used in this specification, such a term should also be construed to also include the exact term. That is, e.g., “substantially straight” should be construed to also include “(exactly) straight”.

Whenever steps were recited in the above or also in the appended claims, it should be noted that the order in which the steps are recited in this text may be accidental. That is, unless otherwise specified or unless clear to the skilled person, the order in which steps are recited may be accidental. That is, when the present document states, e.g., that a method comprises steps (A) and (B), this does not necessarily mean that step (A) precedes step (B), but it is also possible that step (A) is performed (at least partly) simultaneously with step (B) or that step (B) precedes step (A). Furthermore, when a step (X) is said to precede another step (Z), this does not imply that there is no step between steps (X) and (Z). That is, step (X) preceding step (Z) encompasses the situation that step (X) is performed directly before step (Z), but also the situation that (X) is performed before one or more steps (Yi), . . . , followed by step (Z). Corresponding considerations apply when terms like “after” or “before” are used.

Moreover, reference is made to the following specific examples which are given to further illustrate the present invention, without limiting the invention thereto.

### EXAMPLES

### Example 1—Materials and Methods

For the subsequent examples 2-4, the following materials and methods were used: All DNA-origami objects were assembled in 20 mM MgCl₂ using the folding ramp: 15 min at 65° C., 60° C.-45° C. for 1 h/° C. Staple concentrations were 200 nM and scaffold concentrations were 50 nM. Oligonucleotides were obtained from IDT. DNA scaffolds were produced according to Engelhardt et al. [F. A. S. Engelhardt, F. Praetorius, C. H. Wachauf, G. Bruggenthies, F. Kohler, B. Kick, K. L. Kadletz, P. N. Pham, K. L. Behler, T. Gerling, and H. Dietz, ‘Custom-Size, Functional, and Durable DNA Origami with Design-Specific Scaffolds’, ACS Nano (2019), doi 10.1021/acsnano.9b01025]. DNA-origami objects were purified using PEG precipitation [E. Stahl, T. Martin, F. Praetorius, and H. Dietz, ‘Facile and scalable preparation of pure and dense DNA origami solutions’, Angewandte Chemie Intl. Edn., vol 53 (2014), p 12735], before antibody attachment. Purified antibody-DNA conjugates were added to the DNA-origami objects that present the complementary sequences, with a twofold excess per binding site. DNA-origami-antibody objects were purified using PEG precipitation (Stahl et al., ibid.)

All objects were stabilized for flow cytometry and T-cell activation assays using oligolysine-PEG (10 lysine, PEG 5k) as described in [Ponnuswamy, N., Bastings, M., Nathwani, B. et al. Oligolysine-based coating protects DNA nanostructures from low-salt denaturation and nuclease degradation. Nat Commun 8, 15654 (2017). https://doi.org/10.1038/ncomms15654] with N:P ratio of 1:1 (except for logic gate 2, which was incubated at 1:20 ration overnight). Oligolysine-PEG was removed prior to the experiments using 50k-amicon filtration.

T-cell activation was analyzed using a T-Cell Activation Bioassays (Promega, J1655). The experiment was performed according to the instruction of the supplier. Briefly, CD19 expressing target cells (NALM-6) were added to 96 well microtiter plate at a final concentration of 5*105 cells per ml. Then a serial dilution of the different samples (in RPMI 1640 medium) were added. In the end, the genetic modified TCR/CD3 effector cells were added at a final concentration of 1.3*106 cells per ml. The reaction mixture was incubated for 6 h at 37 degree and 5% C02. The genetic modified effector cells express a luciferase intercellularly, if the interleukin 2 promoter is activated. By addition of Bio-Glo reagent, which includes a substrate for the luciferase, the luminescence signal is a direct proportional signal for activation of the TCR/CD3 effector cells, which was analyzed in a microtiter plate reader (Clariostar Plus, BMG).

### Example 2—Conditional Binding-Site Activation by Secondary Binding Event with DNA Origami Logic Gate 1

In order to demonstrate that a binding site's accessibility can be controlled by the occupancy of another binding site, the present inventors developed a block-like DNA-origami object (logic gate 1), consisting of a moveable piston mounted inside a hollow casing (FIG. 21a). The piston's movement is constrained to a linear motion by the shape of the casing. The piston is connected to the case with DNA strands that also serve as adjustable entropic and enthalpic springs. By varying the length of single-stranded or double-stranded sections of the DNA spring (or the combination thereof), it is possible to tune its effective stiffness, thus, rationally adjusting the energy landscape in which the piston travels. The present inventors performed negative-staining TEM of logic gate 1 with a short spring and long stiff spring to image the logic gate 1 in its compressed and extended state, respectively (FIG. 21b). The sequence of the scaffold strand for logic gate 1 is indicated in SEQ ID NO: 1, and the various staple sequences, spring modules and adapter strands for logic gate 1 are indicated in SEQ ID NO: 3 to SEQ ID NO: 137.

Furthermore, the logic gate 1 features binding sites on the piston and on the casing for other objects or molecules (FIG. 21a). The binding sites S_{A} are accessible independent of the logic-gate-1 state, whereas the accessibility of binding site S_{B} depends on the state of the logic gate 1. Specifically, binding site S_{B} has a decreased accessibility in the extended state compared to the compressed state. The present inventors designed logic-gate-1 variants that are predominantly in an extended state, but may be trapped in a compressed state upon binding to another object. Thus upon binding to object A, the logic gate 1 will be trapped in the compressed conformation and the accessibility of the binding site S_{B} will be increased (FIG. 21c). This increase in accessibility will increase the binding affinity to a second object B. In order to test this mechanism, the present inventors constructed plate-like surface mimics A and B from DNA. The present inventors used DNA-strand hybridization as an interaction mechanism. Specifically, surface mimic A has three DNA strands of 24 bases protruding at S_{A′} (FIG. 21c) that are complementary to DNA strands that protrude from the binding sites S_{A} of logic gate 1. Similarly, from surface mimic B protrudes a DNA strand that is complementary to a strand that protrudes from the logic gate's piston at binding site S_{B}. The sequence of the scaffold strand for surface mimics A and B is indicated in SEQ ID NO: 2, and the various staple sequences, spring modules and adapter strands for surface mimics A and B are indicated in SEQ ID NO: 138 to SEQ ID NO: 355.

First, the present inventors wanted to test if the binding of the logic gate 1 to the surface mimic B causes a change in its probability of occupying the compressed or extended state. To this end, the present inventors attached a FRET-dye pair (Cy5 and Cy3) to the piston and case such that the FRET efficiency reports on the state of the logic gate 1. The present inventors prepared logic gates 1 with different DNA springs, mixed these with surface mimics A (excess of surface mimic over logic gate 1), and analysed the mixtures by agarose gel electrophoresis and fluorescence scanning. Mixtures with both, logic gate 1 and surface mimic A, produce a band with slower migration than logic gate 1 only. The present inventors attribute this band to logic-gate-1-surface-mimic-A dimeric objects. Negative-staining TEM imaging of the mixtures also revealed correctly formed dimers (FIG. 21e). The present inventors calculated the FRET efficiency for the logic-gate-1 monomer bands and for the logic-gate-1-surface-mimic-A dimer bands. The FRET efficiency of the logic gate 1 depends on the spring variant and increases once the logic gate 1 binds to surface-mimic A, corroborating the conformational change. The amount of FRET-efficiency change depends on the spring variant.

Next, the present inventors incubated logic-gate-1 variants with surface mimic B or with both surface-mimic A and B and quantified the relative populations at different time points using gel electrophoresis (FIG. 21f). Logic gates carried a cy5 fluorescent dye, whereas surface mimics did not carry any fluorescent dye. Fluorescence imaging of the agarose gels, therefore, visualizes only the logic gate 1 (FIG. 21f). Gels were also post-stained with ethidium bromide to verify the presence of surface mimics. Upon adding the surface mimic B to the logic-gate-1 variants, a slower migrating band occurs whose brightness increases over time. This band corresponds to logic-gate-1-surface-mimic-B dimers (LG-B). For incubations where both surface mimics A and B were added, a dimer band is present already after 1 h of incubation at room temperature, indicating the quick formation of a surface-mimic-A-logic-gate-1 (LG-A) dimer. Besides, a slower migrating band is present that the present inventors attribute to surface-mimic-A-logic-gate-1-surface-mimic-B trimeric objects (LG-A-B). To quantify the binding rates, the present inventors calculated the fraction of LG-B dimers for incubations of logic gate 1 with surface mimic B and the fraction of LG-A-B trimers for incubations of logic gate 1 with surface mimic A and B (FIG. 21g). For all spring variants and all-time points, the LG-A-B trimer yield was higher than the LG-B dimer yield. The presence of surface-mimic A enhances the binding rate of surface-mimic B to the logic gate 1. This activation effect depends on the spring variant. The retracted variant that cannot switch does not show an activation effect, whereas the extended version (21 ds base pairs as springs) shows a significant difference between the non-activated and activated logic gate.

The result obtained in the above experiments are shown in FIG. 21: Conditional binding-site activation on DNA-origami logic gate by a secondary binding event. a, Schematic representation of the logic gate 1 in the extended state (top row) and the compressed state (bottom row). The piston (dark gray) can move inside the case (light gray) and its conformation is controlled using DNA-strand springs (zig-zag lines). Cylinders represent dsDNA helices. Black circles and spheres represent sites where binding moieties are protruding. Schematics on the right are cross sections through the logic gate 1. b, Top and side view of logic-gate-1 average negative-staining TEM particles in extended (top) and compressed (bottom) state. c, Scheme of the conditional binding and activation of binding-site S_{B}. From left to right: logic gate (left) can only bind to surface-mimic A because the binding site S_{B} is inaccessible. Upon binding to the surface-mimic A (middle left), the logic gate can switch (middle right), and present the binding site S_{B}. Finally, the surface-mimic B can bind. d, Image of an agarose gel on which logic gates or logic-gate-surface-mimic-A mixtures were electrophoresed (top). Mixtures included 5 nM logic gate and 10 nM surface-mimic A for 1 h at room temperature with 5 mM MgCl₂. FRET efficiencies of the logic-gate monomer bands (LG) and the logic-gate-surface-mimic-A dimer bands (LG-A) were calculated as described in [J. Funke and H. Dietz, ‘Placing molecules with Bohr radius resolution using DNA origami’, Nature Nanotechnology, (2015), doi 10.1038/nnano.2015.240] (bottom). C: compressed logic gate; E: extended logic gate; F: logic gate without a spring; Tₙ: poly-thymine springs with n=30, 40, or 50 ss thymine bases; TₙdsₘTₙ: spring variants with partly dsDNA segment (m) surrounded with ssDNA thymine bases (n). e, Negative-staining TEM image of logic-gate-surface-mimic-A mixture. Arrows indicate logic-gate-surface-mimic dimers. Scale bar, 100 nm f, Agarose gels on which incubations of logic gates with surface-mimic B (+,−) or with both surface-mimic A and B (+,+) were electrophoresed. Spring variants are labelled as in (e). 5 nM logic gate was incubated with 10 nM surface-mimic B and 10 nM surface-mimic A at room temperature with 5 mM MgCl₂. g, Dashed lines: fraction of logic-gate-surface-mimic-B dimers (LG-B) in incubations of logic gates with surface-mimic B calculated from band intensities of imaged agarose gels. Solid lines: fraction of logic-gate-surface-mimic-B-surface-mimic-A trimers (LG-A-B) in incubations of logic gates with surface-mimic B and with surface-mimic A, calculated from band intensities of imaged agarose gels. Spring variants according to label in d.

### Example 3—Cell Surface Binding Causes Antibody Presentation with Logic Gate 2

In order to demonstrate a binding-site activation mechanism for cells, the present inventors developed a DNA-origami object with a cylindrical shape (FIG. 22a, b, c). The present inventors implemented an opening mechanism via hinges along the cylindrical axis, such that the logic gate 2 is predominantly rolled up. In this closed state, the logic gate features a cavity in which the present inventors mounted two full-sized immune-stimulating anti-CD3 IgG antibodies. The hinge's flexibility and the preferred hinge angle can be rationally designed, such that the logic gate is predominantly in the closed state (rolled up). On the other hand, it can un-roll and adopt a flat conformation (the open state) in which the anti-CD3 antibodies are presented. By using six full-sized anti-CD19 IgG antibodies on the exterior, the logic gate can be trapped in the open state upon multivalent binding to CD19 antigens on cell surfaces. Thus, the immune-stimulating anti-CD3 antibodies are predominantly hidden in solution in the closed state. Upon binding to the CD19-positive target cell, the logic gate is trapped in the open state, and immune-stimulating anti-CD3 antibodies are accessible. This mechanism, therefore, activates the immune-stimulating antibodies only after binding to the target cell. The sequence of the scaffold strand for logic gate 2 is indicated in SEQ ID NO: 2, and the various staple sequences, adapter and hinge module sequences are indicated in SEQ ID NO: 356 to SEQ ID NO: 615.

In order to test this mechanism, the present inventors constructed switchable logic gates 2 that had six anti-CD19 antibodies and non-switchable logic gates 2 that only had two centrally-mounted anti-CD19 antibodies (FIG. 22d). The mechanism relies on trapping the logic gate in the opened state. By only attaching two centrally-mounted anti-CD19 antibodies, the present inventors remove the ability to be trapped in the opened state but retain the target-cell binding affinity. First, the present inventors tested these variants in cell recruiting assays. In particular, the present inventors incubated CD3-positive Jurkat T cells with CD19-positive NALM6 target cells and counted the number of cells in a cell cluster (FIG. 22d, middle). The sample with the non-switchable logic gate variant had a similar fraction of clustered cells as the cell-only control. On the other hand, a variant that was fixed in the open state by 21-bp-long dsDNA strands at the optional-spring positions showed an increased number of cell clusters indicating T-cell recruiting to target cells. Finally, the switchable version had an intermediate cluster fraction, indicating an increased recruiting efficiency over the non-switchable variant, thus indicating the activation of the anti-CD3 antibody upon target-cell binding. The present inventors also tested these variants (non-switchable closed, non-switchable open, and switchable) using T-cell activation assays (FIG. 22d right). The non-switchable open variant had the highest T-cell activation, whereas the non-switchable closed variant had the lowest T-cell activation signal. The flexible switchable variant had an intermediate T-cell activation signal, indicating the increased T-cell binding affinity upon target-cell binding.

One difference between the non-switchable and switchable variant up till now was that the non-switchable variant carried less target-cell binding antibodies. This difference could potentially change the binding rates of the logic gate to the target cell. To rule out that the difference in T-cell activation between the switchable and non-switchable variants was due to a difference in the number of attached antibodies, the present inventors assembled logic-gate variants with the same number of attached antibodies but in different configurations (FIG. 22e left). On the switchable variant, the present inventors placed six anti-CD19 antibodies radially along the exterior. On the non-switchable variant, the present inventors placed six antibodies parallel to the cylindrical axis.

Furthermore, the present inventors increased the target-cell affinity for the non-switchable variant by attaching the antibodies in a more distal configuration, thereby increasing their accessibility. To compare the binding affinity of the constructs, the present inventors incubated the switchable and non-switchable variants with CD19-positive NALM6 B cells and performed flow cytometry (FIG. 22e middle). Independent of the logic-gate conformation (always open or flexible), the non-switchable variant has an increased binding affinity over the switchable variant. The present inventors then performed T-cell activation assays with these variants to investigate the switching mechanism (FIG. 22e right). The T-cell activation signal depends on the hinge type (flexible or always open) and the antibody configuration. For a logic gate variant that was fixed in the open state (always open), the non-switchable antibody configuration yielded an improved T-cell activation over the switchable antibody configuration. This is in agreement with the flow cytometry data because the present inventors expect the non-switchable variant to bind with an increased binding affinity to the target cell, thus causing an increased T-cell activation. Conversely, for flexible logic-gate variants, which are logic gates in the closed state but may adopt open conformations upon target-cell binding, the switchable antibody-configurations had an increased T-cell activation signal. Even though the switchable variants bind weaker to the target cells than the non-switchable variants, the switchable logic gates achieve a higher T cell activation. The present inventors attribute this performance advantage to the increased accessibility of the anti-CD3 antibody of the switchable logic gates after the switching was induced by the target cell.

In summary, even though the switchable logic-gate-2 variant has a disadvantage in binding affinity compared to the non-switchable variant, the switchable variants outperformed the non-switchable variant in T-cell activation by presenting the T-cell activating anti-CD3 antibody after switching. The present inventors used anti-CD19 antibodies to recognize B-cell target cells and anti-CD3 antibodies to cause T-cell activation. However, this mechanism can be extended or adapted to other antibodies, proteins or molecules. In particular, using a combination of different target-cell binding moieties, one could specifically target cells that present a combination of antigens (combination of antigen A and antigen B versus antigen A only versus antigen B only).

The results obtained in the above experiments are shown in FIG. 22: Conditional IgG presentation upon target-cell surface binding. a, Schematic representation of the logic gate 2 in the closed and open state. Cylinders represent dsDNA helices. Y-shaped ellipsoids represent IgG antibodies (light gray=anti-CD3 antibodies, black=anti-CD19 antibodies). Rectangles indicate hinge regions. b, 2D-schematic representation of the hinge region indicated in (a) in closed and open state. Circles represent dsDNA helices. The hinge stiffness can be adjusted using a different number of Thymine base (Tₙ) in the hinge-connections (dotted line) or by using a second optional DNA spring (zig-zag line). Furthermore, the angle theta at which the connection is made can be adjusted to control the resting conformation. c, 3% agarose gel on which logic gates with anti-CD3, anti-CD19, or a combination thereof was electrophoresed. d, Schematic of the antibody configuration for the switchable and non-switchable variant. Middle: fraction of cells in cell clusters for flexible and always open logic gates switchable or non-switchable antibody configurations. Right: T-cell activation signal using NALM6 cells and T Cell Activation Bioassay (Promega J1655) for logic gates variants. e, Median fluorescence from flow cytometry experiments after 1.5 h incubation (middle) and T-cell activation of different logic gate variants after 6 h (Tn indicates number of Thymines in hinge). Both the switchable and the non-switchable antibody configurations had six anti-CD19 antibodies attached.

### Example 4—Cell Surface Binding Causes Antibody Presentation with Logic Gate 3

Using logic gate 2, the present inventors were able to demonstrate the switching on cell surfaces. However, logic gate 2 has five hinges in parallel that all need to rotate in order to switch, carrying up to eight antibodies. Therefore, the present inventors designed logic gate 3 based on the principles of logic gate 2. Logic gate 3 consists of a base section on which two wing-like plates are flexibly mounted (FIG. 23a). The present inventors inserted a unique hinge mechanism between the wings and the base plate. The wings form a 90° angle with the base in the closed conformation and a 180° angle in the open conformation. The hinge's flexibility can be rationally adjusted by adjusting the number of thymine bases at the hinge. An immune-stimulating antibody can be mounted to the base section, such that it is hidden in the closed state and accessible in the open state. Three target-cell specific antibodies can be mounted on the exterior of the logic gate 3. Using different target-cell antibody configurations, the present inventors can construct switchable and non-switchable variants (FIG. 23b, left). For the switchable logic gate, the present inventors mounted one target-cell-binding antibody at the exterior of each wing segment and one at the exterior of the base segment. For the non-switchable variant, all three target-cell-binding antibodies are mounted on the base segment. One antibody is attached at the center and two at the edges of the base segment, to facilitate accessibility. Negative-staining TEM imaging revealed the correct assembly of the logic gate 3, and gel electrophoresis revealed the successful attachment of anti-CD3 and anti-CD19 antibodies to the logic gate. The sequence of the scaffold strand for logic gate 3 is indicated in SEQ ID NO: 1, and the various staple sequences, adapter and hinge module sequences are indicated in SEQ ID NO: 616 to SEQ ID NO: 744.

Flow cytometry of flexible and always open logic gate variants with CD19-positive NALM6 B cells revealed a comparable binding affinity for the switchable and non-switchable antibody configuration (FIG. 23b, right). For the flexible-hinge variant, the binding affinity of the non-switchable antibody-configuration was again higher, thus putting the switchable variant at a disadvantage for T-cell activation. To test the conditional switching of the logic gate 3, the present inventors performed T-cell activation assays with switchable and non-switchable anti-CD19 antibody configurations and NALM6 B cells as target cells (FIG. 23c). For a logic gate that was fixed in the open state, the present inventors observed comparable T cell activation. For flexible-hinge variants, the T cell activation was increased for the switchable antibody-configuration compared to the non-switchable antibody-configuration. At low concentrations, the switchable antibody-configuration outperformed the non-switchable configuration by a factor of two, even though the switchable variant is at a disadvantage due to its decreased binding affinity. The present inventors also studied random T-cell activation using logic gate variants without anti-CD19 target-cell-binding antibodies and found an activation signal only at 1 nM concentration for flexible-hinge variants. The variant that was fixed in the open state showed T cell activation at 300 pM.

In summary, even though the switchable logic-gate-3 variant has a disadvantage in binding affinity compared to the non-switchable variant, the switchable variants outperformed the non-switchable variant in T-cell activation by conditionally presenting the T-cell activating anti-CD3 antibody after switching.

The results obtained in the above experiments are shown in FIG. 23: Conditional IgG presentation upon target-cell surface binding with logic gate 3. a, Schematic representation of the logic gate 3 in the closed and open state. Cylinders represent dsDNA helices. Y-shaped ellipsoids represent IgG antibodies (light gray=anti-CD3 antibodies, black=anti-CD19 antibodies). Rectangles indicate hinge regions. Insets show 2D-schematic representation of the hinge region in closed and open state. Circles represent dsDNA helices. The hinge stiffness can be adjusted using a different number of thymine bases (Tₙ) in the hinge connections (dotted line). b, Left: schematic of the antibody configuration for the switchable and non-switchable variant. Always three anti-CD19 antibodies were attached.

Right: median fluorescence from flow cytometry experiments after 1.5 h incubation for a flexible and always-open variant with different antibody configurations as depicted in the left schematic. c, T-cell activation of different logic gate variants (Tn indicates number n of thymines in hinge). Both the switchable and the non-switchable antibody configurations had three anti-CD19 antibodies attached but in different configurations (as indicated in b, left).

While in the above, embodiments and examples have been described with reference to the accompanying drawings, the skilled person will understand that these embodiments and examples were provided for illustrative purpose only and should by no means be construed to limit the scope of the present invention, which is defined by the claims.

1. A nanostructure (10) comprising:
a binding site configured to bind to a binding target;
wherein the nanostructure is configured to assume a first configuration (A) and a second configuration (B), wherein the accessibility of the binding site for the binding target in the second configuration (B) is different to the accessibility of the binding site for the binding target in the first configuration (A);
wherein the nanostructure comprises a coupling site set comprising at least one coupling site, wherein each coupling site is configured to couple to a respective coupling target;
wherein the nanostructure is configured to assume the second configuration (B) when each coupling site of a subset of the coupling site set is coupled to its respective coupling target, wherein the subset comprises at least one coupling site; and
wherein the nanostructure is configured to assume the first configuration (A) when none of the coupling sites of the coupling site set is coupled to its respective coupling target.

2. The nanostructure according to claim 1, wherein the coupling sites are not configured to couple to each other.

3. The nanostructure according to claim 1, wherein the nanostructure is configured to assume first and second equilibrium states between the first configuration (A) and the second configuration (B),
wherein the probability that the nanostructure assumes the second configuration is different in the second equilibrium state than in the first equilibrium state;
wherein the nanostructure is configured to
assume the first equilibrium state when all of the respective coupling targets are absent; and to
assume the second equilibrium state when all of the respective coupling targets of the subset of coupling sites are present.

4. The nanostructure according to claim 1, wherein the nanostructure comprises a first portion and a second portion;
wherein the first portion is movable with respect to the second portion;
wherein the first portion and the second portion each comprise at least one of the coupling sites of the coupling site set; and
wherein at least one of the first portion and the second portion comprises at least one binding site.

5. The nanostructure according to claim 1, wherein the nanostructure is at least partially formed by a DNA origami structure,
wherein the DNA origami structure comprises at least one scaffolding strand;
wherein the DNA origami structure further comprises a plurality of single-stranded oligonucleotide staple strands;
wherein each staple strand is at least partially complementary to at least one scaffolding strand; and
wherein each of the staple strands is configured to bind to at least one of the at least one scaffolding strand in two distinct places, wherein the at least one scaffolding strand is folded and/or arranged such that a desired nanostructure is formed.

6. The nanostructure according to claim 1, wherein at least one of at least one binding site and at least one coupling site comprised by the coupling site set is a molecule, wherein the molecule is bound to the nanostructure by means of linker molecules.

7. The nanostructure according to claim 5, wherein the molecule is bound to one of the at least one scaffolding strand or a staple strand by means of a linker molecule;
wherein the linker molecule is connected to a DNA strand portion, which is complementary to a portion of the at least one scaffolding strand or to a portion of a staple strand.

8. The nanostructure according to claim 1, wherein the nanostructure comprises a maximum length, and wherein the maximum length is smaller than 1000 nm.

9. A system comprising the nanostructure according to claim 1, wherein the system comprises:
at least one binding target;
a plurality of coupling targets; and
at least one first entity, wherein each of the at least one first entity comprises at least one coupling target.

10. The system according to claim 9, wherein each of the at least one first entity comprises at least one binding target.

11. The system according to claim 9, wherein the system comprises at least one second entity, wherein each of the at least one second entity comprises at least one coupling target.

12. A method for conditionally binding a binding site to a binding target, wherein the method comprises:
utilizing a nanostructure according to claim 1;
the nanostructure assuming the first configuration (A) and the second configuration (B), wherein the accessibility of the binding site for the binding target in the second configuration (B) is different to the accessibility of the binding site for the binding target in the first configuration (A);
wherein the nanostructure assumes the first configuration (A) when none of the coupling sites is coupled to its respective coupling target; and
wherein the nanostructure assumes the second configuration (B) when each of the coupling sites of the subset of the coupling site set is coupled to its respective coupling target.

13. The method according to claim 12, wherein the method comprises;
the nanostructure assuming a first and second equilibrium state between the first configuration (A) of the nanostructure, and the second configuration (B) of the nanostructure, wherein the probability of assuming the second configuration (B) is different in the second equilibrium state than in the first equilibrium state;
the nanostructure assuming the first equilibrium state when all of the respective coupling targets are absent, i.e. none of the coupling sites of the coupling site set can couple to the respective coupling target; and
the nanostructure assuming the second equilibrium state when all of the respective coupling targets of the subset of coupling sites are present, i.e. all coupling sites of the subset of coupling sites can couple to the respective coupling targets.

14. A method for providing therapy, wherein said method comprises administering, to a subject, in need of therapy, a substance comprising a plurality of nanostructures according to claim 1.

15. A composition comprising a plurality of nanostructures according to claim 1 and a pharmaceutical carrier.
